(19) United States
(12) Patent Application Publication
(10) Pub. No.: US 2008/0070867 A1
(43) Pub. Date: Mar. 20, 2008

(54) 2-Imidazolone and 2-Imidazolidinone Heterocyclic Inhibitors of Tyrosine Phosphatases

(75) Inventors: Zacharia S. Cheruvallath, San Diego, CA (US); Joseph Semple, San Diego, CA (US); Vasanthakumar Rajappan, San Diego, CA (US); Darryl Rideout, San Diego, CA (US); Venkatachalapathi Yalamoori, San Diego, CA (US); Chung-Ying Tsai, San Diego, CA (US); Feiyue Wu, Poway, CA (US); Lars Thoresen, San Diego, CA (US); Mark Shenderovich, San Diego, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Metabasis Therapeutics, Inc., La Jolla, CA (US)

(21) Appl. No.: 11/814,239

(22) PCT Filed: Jan. 17, 2006
(86) PCT No.: PCT/US06/01708

§ 371 Date: Jul. 31, 2007

Related U.S. Application Data
(60) Provisional application No. 60/645,535, filed on Jan. 19, 2005.
(60) Provisional application No. 60/714,136, filed on Sep. 2, 2005.

(51) Int. Cl. A61K 31/675; A61P 3/10; C07F 9/02

(52) U.S. Cl. 514/92; 548/112

(57) ABSTRACT

Compounds and compositions are provided for modulating the activity of protein tyrosine phosphatases. The compounds for use in the compositions and methods provided herein have formulae (I) Protein tyrosine phosaphatase, including PTB-IB, mediated diseases and disorders include diabetes including Type 1 and Type 2 diabetes (and associated complications such as hypertension, ischemic diseases of the large and small blood vessels, blindness, circulatory problems, kidney failure and atherosclerosis), syndrome X, metabolic syndrome, glucose intolerance, insulin resistance, obesity, cancer, and neurodegenerative diseases.

### RELATED APPLICATIONS

Benefit of priority to U.S. provisional patent application Nos. 60/645,535, filed Jan. 19, 2005, entitled “2-Imidazolone Heterocyclic Inhibitors Of Tyrosine Phosphatases,” and 60/714,136, filed Sep. 2, 2005, entitled “2-Imidazolone and 2-Imidazolidinone Heterocyclic Inhibitors of Tyrosine Phosphatases,” is claimed herein. The disclosures of the above-referenced provisional applications are incorporated herein by reference in their entirety.

### FIELD

Provided herein are methods of inhibiting the activity of tyrosine phosphatases that regulate signal transduction, and, more particularly, use of 2-imidazolone and 2-imidazolidinone heterocyclic compounds and compositions as tyrosine phosphatase inhibitors for the treatment of conditions and diseases that respond to phosphatase inhibition.

### BACKGROUND

Cellular signal transduction is a fundamental mechanism whereby external stimuli that regulate cellular processes are relayed to the interior of cells. The biochemical pathways through which signals are transmitted within cells comprise a circuitry of directly or functionally connected interactive proteins. One of the key biochemical mechanisms of signal transduction involves the reversible phosphorylation of tyrosine residues on proteins. The phosphorylation state of a protein may affect its conformation and/or enzymatic activity as well as its cellular location. The phosphorylation state of a protein is modified through the reciprocal actions of protein tyro sine kinases (PTKs) and protein tyro sine phosphatases (PTPs) at various specific tyrosine residues.

A common mechanism by which receptors regulate cell function is through an inducible tyrosine kinase activity which is either endogenous to the receptor or is imparted by other proteins that become associated with the receptor (Darnell et al., 1994, Science 264:1415-1421; Heldin, 1995, Cell 80:213-223; Pawson, 1995, Nature 373:573-580).

Protein tyrosine kinases comprise a large family of transmembrane receptor and intracellular enzymes with multiple functional domains (Taylor et al., 1992, Ann. Rev. Cell Biol. 8:429-62). The binding of ligand allosterically transduces a signal across the cell membrane where the cytoplasmic portion of the PTKs initiates a cascade of molecular interactions that disseminate the signal throughout the cell and into the nucleus.

Like the PTKs, the protein tyrosine phosphatases (PTPs) comprise a family of transmembrane and cytoplasmic enzymes, possessing at least an approximately 230 amino acid catalytic domain containing a highly conserved active site with the consensus motif >I/V!HCXAGXXR>S/T!G. The substrates of PTPs may be PTKs which possess phosphotyrosine residues or the substrates of PTKs (Hunter, 1989, Cell 58:1013-16; Fischer et al, 1991, Science 253:401-6; Saito & Streuli, 1991, Cell Growth and Differentiation 2:59-65; Pot and Dixon, 1992, Biochem. Biophys. Acta 1136:35-43).

Transmembrane or receptor-like PTPs (RPTPs) possess an extracellular domain, a single transmembrane domain, and one or two catalytic domains followed by a short cytoplasmic tail. The extracellular domains of these RPTPs are highly divergent, with small glycosylated segments (e.g., RPTPa, RPTPe), tandem repeats of immunoglobulin-like and/or fibronectin type III domains (e.g., LAR) or carbonic anhydrase like domains (e.g., RPTPg, RPTPb). These extracellular features might suggest that these RPTPs function as a receptor on the cell surface, and their enzymatic activity might be modulated by ligands. Intracellular or cytoplasmic PTPs (CPTPs), such as PTP1C, PTP1D, typically contain a single catalytic domain flanked by several types of modular conserved domains. For example, PTP1C, a hemopoietic cell CPTP, is characterized by two Src-homology homology 2 (SH2) domains that recognize short peptide motifs bearing phosphotyrosine (pTyr).

In general, these modular conserved domains influence the intracellular localization of the protein. SH2-containing proteins are able to bind pTyr sites in activated receptors and cytoplasmic phosphoproteins. Another conserved domain known as SH3 binds to proteins with proline-rich regions. A third type known as pleckstrin-homology (PH) domain has also been identified. These modular domains have been found in both CPTKs and CPTPs as well as in non-catalytic adapter molecules, such as Grbs (Growth factor Receptor Bound), which mediate protein-protein interactions between components of the signal transduction pathway (Skolnik et al., 1991, Cell 65:83-90; Pawson, 1995, Nature 373:573-580).

Multiprotein signaling complexes comprising receptor subunits, kinases, phosphatases and adapter molecules are assembled in subcellular compartments through the specific and dynamic interactions between these domains with their binding motifs. Such signaling complexes integrate the extracellular signal from the ligand-bound receptor and relay the signal to other downstream signaling proteins or complexes in other locations inside the cell or in the nucleus (Koch et al., 1991, Science 252:668-674; Pawson, 1994, Nature 373:573-580; Mauro et al., 1994, Trends Biochem Sci 19:151-155; Cohen et al., 1995, Cell 80:237-248).

The levels of tyrosine phosphorylation required for normal cell growth and differentiation at any time are achieved through the coordinated action of PTKs and PTPS. Depending on the cellular context, these two types of enzymes may either antagonize or cooperate with each other during signal transduction. An imbalance between these enzymes may impair normal cell functions leading to metabolic disorders and cellular transformation.

For example, insulin binding to the insulin receptor, which is a PTK, triggers a variety of metabolic and growth promoting effects such as glucose transport, biosynthesis of glycogen and fats, DNA synthesis, cell division and differentiation. Diabetes mellitus, which is characterized by insufficient or a lack of insulin signal transduction, can be caused by any abnormality at any step along the insulin signaling pathway (Olefsky, 1988, in “Cecil Textbook of Medicine,” 18th Ed., 2:1360-81).

Relatively less is known with respect to the direct role of tyrosine phosphatases in signal transduction; PTPs may play a role in human diseases. For example, ectopic expression of RPTPa produces a transformed phenotype in embryonic fibroblasts (Zheng et al., Nature 359:336-339), and overexpression of RPTPa in embryonal carcinoma cells causes the cells to differentiate into a cell type with neuronal phenotype (den Hertog et al., EMBO J. 12:3789-3798). The gene for human RPTPg has been localized to chromosome 3p21 which is a segment frequently altered in renal and small lung carcinoma. Mutations may occur in the extracellular segment of RPTPg which result in RPTPs that no longer respond to external signals (LaForgia et al., Wary et al., 1993, Cancer Res 52:478-482). Mutations in the gene encoding PTP1C (also known as HCP, SHP) are the cause of the motheaten phenotype in mice which suffer severe immunodeficiency, and systemic autoimmune disease accompanied by hyperproliferation of macrophages (Schultz et al., 1993, Cell 73:1445-1454). PTP1D (also known as Syp or PTP2C) has been shown to bind through SH2 domains to sites of phosphorylation in PDGFR, EGFR and insulin receptor substrate 1 (IRS-1). Reducing the activity of PTP1D by microinjection of anti-PTP1D antibody has been shown to block insulin or EGF-induced mitogenesis (Xiao et al., 1994, J Biol Chem 269:21244-21248).

Thus, there is a need for compounds and compositions that modulate the activity of tyrosine phosphatases, including PTP-1B.

### SUMMARY

Provided herein are compounds and compositions for modulation of tyrosine phosphatase activity. In one embodiment, compounds and compositions for inhibiting protein tyrosine phosphatase activity are provided. In another embodiment, provided herein are compounds and compositions that are useful in the treatment, prevention, or amelioration of one or more symptoms of diseases caused by dysfunctional signal transduction, or in which dysfunctional signal transduction is implicated. In another embodiment, provided herein are compounds and compositions for treatment, prevention, or amelioration of one or more symptoms of diabetes.

In one embodiment, the compounds for use in the compositions and methods provided herein have formulae I:

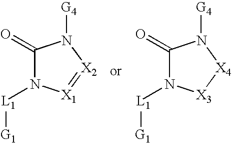

where X₁ and X₂ are each, independently, N, CG₂ or CG₃, with the proviso that only one of X₁ and X₂ can be N;

X₃ and X₄ are each, independently, NR or CG₂G₃, where R is H, alkyl, alkenyl, alkynyl, cycloalkyl, heterocyclyl, aryl or heteroaryl, with the proviso that only one of X₃ and X₄ can be NR;

L₁ is a linker;

G₁ is:

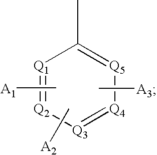

G2 and G3 are selected from (i) or (ii) as follows:

(i) G₂ is H, alkyl or:

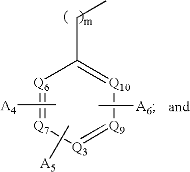

G₃ is H, alkyl, alkoxy, F, Br, Cl or:

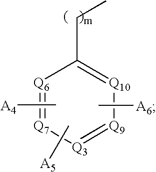

or (ii) G2 and G3, together with the atoms to which they are attached, form a 5-7 membered cycloalkyl, heterocyclyl, aryl or heteroaryl ring;

G₄ is H, alkyl, cycloalkyl, COOalkyl, cycloalkylalkyl or:

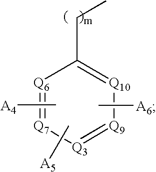

m is an integer from 0 to 2;

Q₁ through Q₁₀ are independently selected from no bond (direct link), C, N, S, and O, with the proviso that the resulting combination of atoms is a chemically stable cyclic and/or (hetero)aromatic ring system, and where if the Q is C, then it is substituted with one of A1-A6 or hydrogen, and where if the Q is N, S or O, it is not substituted with one of A1-A6; and

where A1-A6 are independently selected from:

(i) no substituent, H, F, Cl, Br, I, CF₃, CF₂CF₃, CH₂CF₃, CF₂CH₃, OH, OCF₃, OCHCl₂, CN, NO₂, C₁-C₆-alkyl which is unsubstituted or mono-, di- or tri-substituted with 1 to 3 substituents selected from the group consisting of Y₁, Y₂, and Y₃, C₂-C₆-alkenyl which is unsubstituted or mono-, di- or tri-substituted with 1 to 3 substituents selected from the group consisting of Y₁, Y₂, and Y₃, C₂-C₆-alkynyl which is unsubstituted or mono-, di- or tri-substituted with 1 to 3 substituents selected from the group consisting of Y₁, Y₂, and Y₃, C₁-C₆ alkoxy which is unsubstituted or mono-, di- or tri-substituted with 1 to 3 substituents selected from the group consisting of Y₁, Y₂, and Y₃, C₃-C₆ alkenyloxy which is unsubstituted or mono-, di- or tri-substituted with 1 to 3 substituents selected from the group consisting of Y₁, Y₂, and Y₃, C₃-C₆ alkynyloxy which is unsubstituted or mono-, di- or tri-substituted with 1 to 3 substituents selected from the group consisting of Y₁, Y₂, and Y₃, wherein Y₁, Y₂, and Y₃ are defined above, C₃-C₈-cycloalkyl which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃; two adjacent A groups (e.g. A₁, A₂) may be joined together to form a fused alicyclic, heteroaromatic or aromatic ring. R, R1=H, C₁-C₆-alkyl, C₂-C₆-alkenyl, C₂-C₆-alkynyl; R, R1 may be joined together to form an alicyclic or heterocyclic ring; and one or more of A₁-A₆ may serve as a linking atom, such as O, S(O)₀₋₂, C(RR1), P(═O), P(═S), or N(R);

(ii) A₁-A₆ phosphorous-containing moieties including the following:

P(═O)(OR)(OR1), especially P(═O)(OH)₂, P(═O)(OH)(OCH₃), P(═O)(OH)(OC₂H₅), P(═O)(OR)[(OCRR1)OC(═O)R], P(═O)(OR)[(OCRR1)OC(═O)OR], P(═O)[(OCRR1)OC(═O)R)]₂, P(═O)[(OCRR1)OC(═O)OR)]₂, P(═O)(OR)(OR1), P(═O)(Me)(OR), P(═O)(CF₃)(OR), P(═O)(Me)(NHR), P(═O)(NHR)(OR), P(═O)(NHR)(NHR1), CR═CR—P(═O)(OR)(OR1), CR═CR—P(═O)(Me)(OR), CR═CR—P(═O)(CF₃)(OR), CR═CR—P(═O)(Me)(NHR), CR═CR—P(═O)(NHR)(OR), CR═CR—P(═O)(NHR)(NHR1), [CH(OH)]_{q}P(═O)(OR)(OR1), [CH(OH)]_{q}P(═O)(Me)(OR1), [CH(OH)]_{q} P(═O)(CF₃)(OR1), CC—P(═O)(OR)(OR1), CC—P(═O)(Me)(OR), CC—P(═O)(CF₃)(OR), CC—(CF₂)_{q}—P(═O)(OR)(OR1), CC—(CF₂)_{q}—P(═O)(Me)(OR1), CC—(CF₂)_{q}—P(═O)(CF₃)(OR1), [CH(OH)]_{q}CF₂P(═O)(OR)(OR1), [CH(OH)]_{q}(CF₂)_{q}P(═O)(Me)(OR1), [CH(OH)]_{q}(CF₂)_{q}P(═O)(CF₃)(OR1), (CF₂)_{q} P(═O)(OR)(OR1), (CF₂)_{q}P(═O)(Me)(OR), (CF₂)_{q}P(═O)(CF₃)(OR), (CF₂)_{q} P(═O)(Me)NHR, (CF₂)_{q}P(═O)(NHR)(OR), (CFR)_{q}P(═O)(OR)(OR1), (CFR)_{q}P(═O)(Me)(OR), —(CFR)_{q}P(═O)(CF₃)(OR), (CFR)_{q}P(═O)(Me)NHR, (CF₂)_{q}P(═O)(NHR)(OR), CF═CF—P(═O)(OR)(OR1), CF═CF—P(═O)(Me)(OR), CF═CF—P(═O)(CF₃)(OR), CF═CF—P(═O)(Me)(NHR), CF═CF—P(═O)(NHR)(OR), CH═C [P(═O)(OR)₂]₂, CF═C[P(═O)(OR)₂]₂,CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)₂]₂, CF[P(═O)(OR)(OR1)]₂, CF[P(═O)(Me)(OR)]₂, CF[P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, and C(OH)[P(═O)(NHR)(OR)]₂, wherein each q is, independently, 1 to 3 throughout;

(iii) A₁-A₆ sulfur-containing moieties including the following:

SO₃H, SO₂NH₂, SO₂NHTz1, SO₂NHC(═O)(Me, CF₃), SO₂NHC(═O)NH₂, (CRR1)_{q}SO₃H, (CRR1)_{q} SO₂NH₂, (CRR1)_{q}SO₂NHTz1, (CRR1)_{q}SO₂NHC(═O)(Me, CF₃), (CRR1)_{q}SO₂NHC(═O)NH₂, SO₂NHCRR1C(═O)C(═O)OR, SO₂CF₃, CH(SO₂Me)₂, CH(SO₂CF₃)₂, SO₂CRR1C(═O)OR, SO₂CH[C(═O)OR]₂, (CRR1)_{q}SO₂NHCRR1C(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tz1, SO₂(CRR1)_{q}P(═O)(OR)₂, SO₂(CF₂)_{q}C(═O)OR, SO₂(CF₂)_{q}Tz1, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO₂NHC(═O)(Me, CF₃), and C(═NSO₂CF₃)(NHSO₂CF₃);

(iv) A₁-A₆ nitrogen-containing moieties including the following:

NHC(═O)C(═O)OR, NHC(═O)C(═O)O(CRR1)OC(═O)R, NHC(═O)C(═O)O(CRR1)OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTz1, NHC(═O)Tz1, NHSO₂Tz1, NH(CF₂)_{q}Tz1, NHSO₂(CRR1)_{q}C(═O)OR, NHSO₂(CF₂)_{q}C(═O)OR, (CRR1)_{q}NO₂, (CF₂)_{q}NO₂, CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR]CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), and NH(CFR)_{q}P(═O)(CF₃)(OR);

(v) A₁-A₆ carbonyl-containing moieties including the following:

C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂, CH═C[C(═O)OR]₂, CF═C[C(═O)OR]₂, C(R4)═C(R5)(R6), (where R4, R5=H, Me, anionic groups, including OH, SO₃H, carboxyl, tetrazole, 3-hydroxy-isoxazol-5-yl, C(═O)NHSO₂(Me, CF₃), C(═O)NHC(═O)(Me, CF₃), SO₂NHC(═O)(Me, CF₃), R6═H, F), C(═O)C(═O)OR, C(═O)CH[C(═O)OR]₂, C(═O)CH(Tz1)₂, C(═O)CRR1C(═O)(Me, CF₃, Ph), C(═O)CRR1SO₂(Me, CF₃, Ph), (CRR1)_{q}C(═O)C(═O)OR, (CF₂)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, CHFC(═O)CF₃, CHFC(OR)₂CF₃, CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C[C(═O)OH]=NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tz1)₂, C(═O)N[CRR1P(═O)(OR)₂]₂, and C(═O)NHC(CRR1OR)₃;

(vi) A₁-A₆ tetrazole (Tz1)-containing moieties including the following:

Tz1, CR(Tz1)₂, (CRR1)_{q}Tz1, (CF₂)_{q}Tz1, (CFR)_{q}Tz1, CF(Tz1)₂, (CF₂)_{q}CF(Tz1)₂, (CF₂)_{q}CR(Tz1)₂, CR═CR-Tz1, CF═CH-Tz1, CH═CF-Tz1, CF═CF-Tz1, CH═C(Tz1)₂, CF═C(Tz1)₂, C(H, F)═C(Tz1)[P(═O)(OR)(OR1), P(═O)(Me)(OR), P(═O)(CF₃)(OR), P(═O)(Me)(NHR), P(═O)(NHR)(OR), C(═O)OR];

(vii) A₁-A₆ oxygen-containing or oxygen-linked moieties including the following:

OH, OR, O(CRR1)_{q}C(═O)OR, O(CF₂)_{q}C(═O)OR, OCH[C(═O)OR]₂, O(CRR1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTz1, O(CRR1)_{q}Tz1, O(CF₂)_{q}Tz1, OCH(Tz1)₂, O(CF₂)_{q}CF(Tz1)₂, O(CF₂)_{q}CR(Tz1)₂, OCF(Tz1)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(NHR)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR1)_{q}P(═O)(NHR)(OR), O(CRR1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF[P(═O)(OR)(OR1)]₂, OCF[P(═O)(Me)(OR)]₂, OCF[P(═O)(CF₃)(NHR)]₂, OCF[P(═O)(NHR)(OR)]₂, O(CRR1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(NHR), O(CRR1)_{q}(CF₂)_{q}P(═O)NHR)(OR), ON═CH—C(═O)OR, and ON═C[C(═O)OR]CRR1C(═O)OR.

(viii) other A₁-A₆ moieties containing the following heteroaryl, squarate, and related derivatives, including: 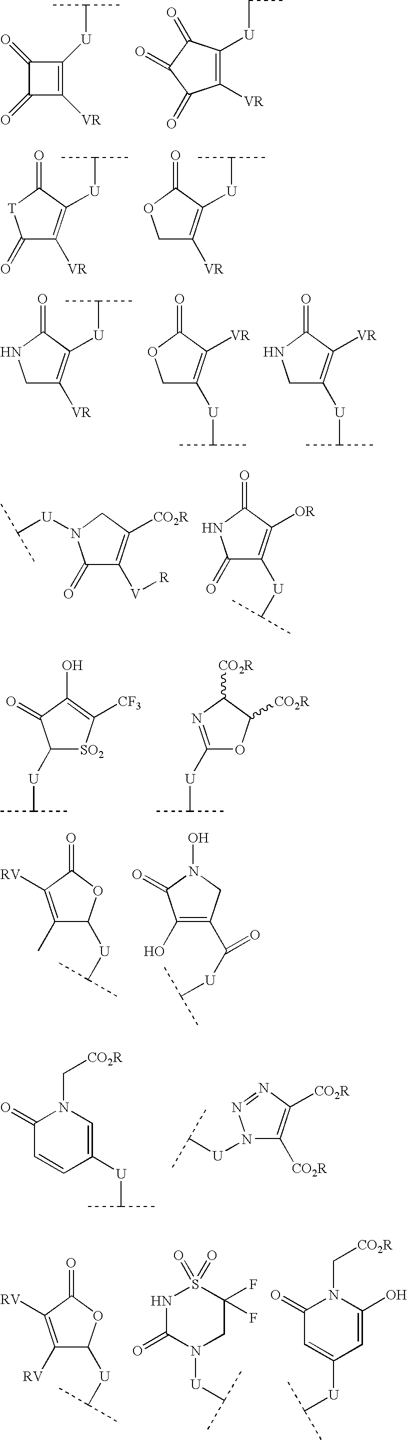 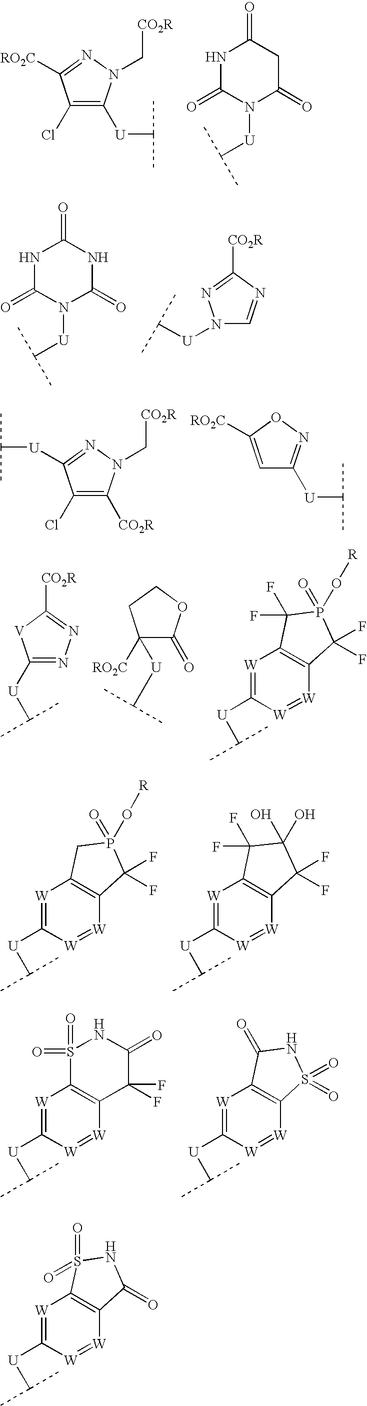
wherein T=O, NR1, CR; U and V are chosen from direct link, (CRR1)_{q}, O, S, NR1; W═CR, N; and R and R1 are as defined above; or

A₁ through A₆ substituent groups can be combined to form stable mono- or bicyclic-fused alicyclic, heterocyclic and/or (hetero)aromatic rings.

Also provided are pharmaceutically-acceptable derivatives, including salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Further provided are pharmaceutical compositions containing the compounds provided herein and a pharmaceutically acceptable carrier. In one embodiment, the pharmaceutical compositions are formulated for single dosage administration.

Methods of modulating protein tyrosine phosphatase, including PTP-1B, using the compounds and compositions provided herein are provided. Further provided are methods of inhibiting protein tyrosine phosphatase, including PTP-1B, using the compounds and compositions provided herein. Also provided are methods of increasing insulin sensitivity using the compounds and compositions provided herein. Methods of treating, preventing, or ameliorating one or more symptoms of protein tyrosine phosphatase, including PTP-1B, mediated diseases are also provided.

Protein tyrosine phosphatase, including PTP-1B, mediated diseases and disorders include, but are not limited to, diabetes including Type 1 and Type 2 diabetes (and associated complications such as hypertension, ischemic diseases of the large and small blood vessels, blindness, circulatory problems, kidney failure and atherosclerosis), syndrome X, metabolic syndrome, glucose intolerance, insulin resistance, leptin resistance, obesity, cancer, neurodegenerative diseases, and other diseases in which the activity of a tyrosine phosphatase or multiple tyrosine phosphatases contributes to the symptoms or pathology thereof.

In practicing the methods, effective amounts of the compounds or compositions containing therapeutically effective concentrations of the compounds are administered.

Articles of manufacture are provided containing packaging material, a compound or composition provided herein which is useful for treating, preventing, or ameliorating one or more symptoms of protein tyrosine phosphatase, including PTP-1B, mediated diseases or disorders, and a label that indicates that the compound or composition is useful for treating, preventing, or ameliorating one or more symptoms of protein tyrosine phosphatase, including PTP-1B, mediated diseases or disorders.

### DETAILED DESCRIPTION

### A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, protein tyrosine phosphatase (PTP) refers to an enzyme of the PTP class, including enzymes that are both tyrosine-specific and dual-specific in their phoshpatase activity. In one embodiment, such phosphatases encompass both transmembrane receptor-like PTPs (RPTPs) as well as soluble cytosolic proteins. RPTPs include small glycosylated segments (e.g., RPTPa, RPTPe), tandem repeats of immunoglobulin-like and/or fibronectin type III domains (e.g., LAR) or carbonic anhydrase like domains (e.g., RPTPg, RPTPb). Intracellular or cytoplasmic PTPs (CPTPs), include PTP1B or PTP-1B, PTP1C and PTP1D, and typically contain a single catalytic domain flanked by several types of modular conserved domains.

As used herein, protein tyrosine phosphatase 1 B (PTP-1B) refers to a 37-kD protein comprised of a single domain, is topologically organized into 8 alpha helices and 12 beta sheets. See, e.g., Jia, Z., Barford, D., Flint, A. J., and N. K. Tonks (1995) Science 268:1754-1758; Pannifer A., Flint A., Tonks N., and Barford D. (1998) The Journal of Biological Chemistry 273:10454-10462.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, aralkyl and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which (X-synuclein fibril formation is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of α-synuclein fibril formation, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392). Other prodrugs for use herein are described elsewhere herein.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is (are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is (are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR), —S(═O)—(CRR)ₙ—, and —(CRR), —S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Y1.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

The compounds provided herein modulate the activity, in one embodiment inhibit the activity, of protein tyrosine phosphatases, including PTP-1B. The compounds have formulae I wherein the substituents are as described elsewhere herein. The substituents are described in greater detail below. All combinations of the descriptions for each substituent are considered to be within the scope of the instant disclosure.

### 1. L₁

In one embodiment, the compounds for use in the compositions and methods provided herein have formulae I, where L₁ is a bond, CH₂, CH₂CH₂, CRR₁, or C(RR₁)C(RR₁),

where R and R1 are independently selected from hydrogen, an alkyl group of 1 to 6 carbon atoms, where the alkyl group is unsubstituted or mono-, di- or tri-substituted with 1 to 3 substituents selected from the group consisting of Y₁, Y₂, Y₃, an aryl group, —OC(R2R3)OC(═O)R4, and —OC(R2R3)OC(═O)OR4, or where R and R1 are joined to form a 4-8 membered cycloalkyl, cycloalkenyl, cycloalkynyl, or heterocyclic ring;

where R2, R3 and R4 are independently selected from (i) and (ii) as follows:

(i) H, C₁-C₇ alkyl, alkenyl of 2 to 6 carbon atoms, where the alkenyl group is unsubstituted or mono-, di- or tri-substituted with 1 to 3 substituents selected from the group consisting of Y₁, Y₂, and Y₃, alkynyl of 2 to 6 carbon atoms, where the alkynyl group is unsubstituted or mono-, di- or tri-substituted with 1 to 3 substituents selected from the group consisting of Y₁, Y₂, and Y₃, cycloalkyl of 3 to 8 carbon atoms, where the cycloalkyl group is unsubstituted or mono-, di- or tri-substituted with 1 to 3 substituents selected from the group consisting of Y₁, Y₂, and Y₃, aryl of 6 to 14 carbon atoms, where the aryl group is unsubstituted or mono-, di- or tri-substituted with 1 to 3 substituents selected from the group consisting of Y₁, Y₂, and Y₃, linked biaryl or heterobiaryl groups of 10 to 20 atoms featuring two aromatic or heteroaromatic ring systems linked through a single bond, with the ring atoms selected from carbon and heteroatoms, where the heteroatoms are selected from oxygen, nitrogen, and sulfur, and where the linked biaryl or heterobiaryl group is unsubstituted or mono-, di- or tri-substituted with 1 to 3 substituents selected from the group consisting of Y₁, Y₂, and Y₃, aralkyl of 7 to 16 carbon atoms, where the aralkyl is unsubstituted or mono-, di- or tri-substituted with 1 to 3 substituents selected from the group consisting of Y₁, Y₂, and Y₃, monocyclic-heteroaryl or bicyclic-heteroaryl having 5 to 14 ring atoms with the ring atoms selected from carbon and heteroatoms, where the heteroatoms are selected from oxygen, nitrogen, and sulfur, and where the monocyclic-heteroaryl or bicyclic heteroaryl group is unsubstituted or mono-, di- or tri-substituted with 1 to 3 substituents selected from the group consisting of Y₁, Y₂, and Y₃, and a heteroaralkyl group of 5 to 14 ring atoms with the ring atoms selected from carbon and heteroatoms, where the heteroatoms are selected from oxygen, nitrogen, and sulfur, where the heteroaralkyl is unsubstituted or substituted on the alkyl chain and which is unsubstituted on the ring or mono-, di- or tri-substituted on the ring with 1 to 3 substituents selected from the group consisting of Y₁, Y₂, and Y₃; or

(ii) R2 and R3, and/or R3 and R4, and/or R2 and R4 are joined to form a 4-8-membered cycloalkyl, cycloalkenyl, cycloalkynyl, or heterocyclic ring, and the other of R2, R3, and R4, when not joined in a ring, is selected as in (i) above;

and wherein Y1, Y2, and Y3 are independently selected from (i) or (ii) as follows:

(i) R5, (CR5R6)ₙOR5, OH, (CR5R6)ₙNR5R6, C(═NR5)NR5R6, C(═NOR5)NR5R6, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5, C(═O)R5, —(CR5R6)ₙC(═O)R5, (CF₂)ₙC(═O)R5, (CFR5)ₙC(═O)R5, tetrazolyl (Tz1), (CR5R6)ₙTz1, (CF₂)ₙTz1, (CFR5)ₙTz1, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, (CR5R6)ₙC(═O)C(═O)OR5, (CR5R6)ₙCH(OR5)C(═O)OR5, C(R5)═C(R6), C(═O)OR5, C(R5)═C(R6)-Tz1, (CR5R6)ₙP(═O)(OH)₂, (CR5R6)ₙP(═O)(OR5)(OR6), P(═O)(OR5)[(OCR5R6)OC(═O)R5], P(═O)(OR5)[(OCR5R6)OC(═O)OR5], P(═O)[(OCR5R6)OC(═O)R5)][(OCR5R6)OC(═O)R5], P(═O)[(OCR5R6)OC(═O)OR5)][(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5=CR5-P(═O)(OR5)(OR6), CR5=CR5-P(═O)(Me)(OR5), CC—P(═O)(OR5)(OR6), (C═O)P(═O)(OR5)(OR6), (C═O)P(═O)(Me)(OR5), (C═O)P(═O)(CF₃)(OR5), (CR5OR6)ₙP(═O)(OR5)(OR6), (CR5OR6)ₙP(═O)(Me)(OR5), (CR5OR6)ₙP(═O)(CF₃)(OR5), O(CR5R6)ₙC(═O)OR5, O(CF₂)ₙC(═O)OR5, OCH[C(═O)OR5]₂, O(CR5R6)ₙCH[C(═O)OR5]₂, OCF[C(═O)OR5]₂, O(CR5R6)ₙC(═O)C(═O)OR5, O(CF₂)ₙC(═O)C(═O)OR5, O(CR5R6)ₙTz1, O(CF₂)ₙTz1, OCH(Tz1)₂, O(CF₂)ₙP(═O)(OR5)(OR6), O(CF₂)—P(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF[P(═O)(Me)(OR5)]₂, SO₃H, —(CR5R6)ₙSO₃H, S(O)ₙR5, SCF₃, SCHF₂, SO₂CF₃, SO₂Ph, (CR5R6)ₙS(O)ₙR5, (CR5R6)ₙS(O)₂CF₃, (CR5R6)ₙSO₂NR5R6, (CR5R6)ₙSO₂NR5C(═O)(Me, CF₃), (CF₂)ₙSO₃H, (CFR5)ₙSO₃H, and (CF₂)ₙSO₂NR5R6, where n=0-2, and where R5 and R6 can be H, C1-C6 alkyl, C2-C6 alkenyl, C2-C6 alkynyl, a C3-C8 cycloalkyl ring, or a 5-7 membered heterocyclic ring; or

(ii) Y₁ and Y₂, and/or Y₁ and Y₃, and/or Y₂ and Y₃ are selected together to be (CR5R6)₂₋₆, —O[C(R8)(R9)]ᵣO— or —O[C(R8)(R9)]ᵣ₊₁-, where r is an integer from 1 to 4 and R8 and R9 are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroarylalkyl of 5 to 14 ring atoms, and the other of Y1, Y2, and Y3, when not selected as in (ii), is selected as in (i) above.

In another embodiment, L₁ is CH₂CH₂ or CH₂. In another embodiment, L₁ is CH₂.

### 2. X₁, X₂, X₃ and X₄

In another embodiment, X₁ and X₂ are CR₇ or N, where R₇ is H, C₁-C₃ alkyl; fluoro, chloro or bromo. In another embodiment, X₁ and X₂ are each independently CG₂ or CG₃. In another embodiment, X₁ is CG₂. In another embodiment, X₂ is CG₃.

In another embodiment, X₃ and X₄ are CG₂G₃. In another embodiment, X₃ is CG₂G₃. In another embodiment, X₃ is CHG₂. In another embodiment, X₄ is CH₂.

### 3. G1

In another embodiment, G₁ is a phenyl ring, where the phenyl ring is substituted with one or more moieties, in one embodiment one moiety, selected from A1-A3. In another embodiment, G₁ is selected from the group consisting of phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester, where acyl is C₂-C₇ alkanoyl or C4-C7 cycloalkanoyl, and phosphonodifluoromethyl mono-alkoxycarbonyloxymethyl ester, where alkoxy is C1-C6 or C3-C6 cycloalkoxy;

and is optionally further substituted with one or more groups selected from 2-carboxyethenyl optionally substituted with 1-2 fluorines or methyl groups, carboxymethoxy, carboxy-C2-C4-alkyl optionally further substituted with 1-4 halogen atoms or 1-4 methyl groups, Cl, Br, F, I, CN, OH, CH₃, and ethynyl.

In another embodiment, G₁ is a phenyl ring substituted with phosphonodifluoromethyl, and is further substituted with Cl, Br, F, CN, OH, CMe₃, CH₃ or ethynyl. In another embodiment, G₁ is a phenyl ring substituted with phosphonodifluoromethyl, and is further substituted with Cl or Br. In another embodiment, G₁ is a phenyl ring substituted with phosphonodifluoromethyl, and is further substituted with Br.

In another embodiment, G₁ is 4-phosphonodifluoromethyl-3-bromophenyl or 4-phosphonodifluoromethyl-3-chlorophenyl.

### 4. G₂

In another embodiment, G₂ is H, C₁-C₃ alkyl, or a phenyl, C₁₋₃alkylenephenyl or heteroaryl, in one embodiment a 5-membered heteroaryl, ring, where the phenyl, C₁₋₃alkylenephenyl or heteroaryl ring is optionally substituted with A4-A6. In another embodiment, G₂ is optionally substituted with:

(i) F, Cl, Br, CN, CF₃, OR, carboxy, alkylenedioxy, alkylene where the alkylene group forms a fused bicyclic group with the phenyl, C₁₋₃alkylenephenyl or heteroaryl ring, (CRR₁)ₙCO₂R, CF₂CO₂R, O(CRR₁)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)CO₂R, C₁-C₃-alkylsulfonyl, or CF₂P(═O)(OR)(OR1);

(ii) phenyl, where the phenyl is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1);

(iii) phenoxy, where the phenoxy is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1); or

(iv) benzyloxy, where the benzyloxy is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1), where m=0 to 6 and n=0 to 2.

In another embodiment, G₂ is H, C₁-C₃ alkyl, or a phenyl, benzyl, thienyl or pyridyl ring, where the phenyl, benzyl, thienyl or pyridyl ring is optionally substituted with (i)-(iv), above. In another embodiment, G₂ is a phenyl or thienyl ring, where the phenyl or thienyl ring is optionally substituted with (i)-(iv), above. In another embodiment, G₂ is a thienyl ring.

In another embodiment, G₂ is a phenyl ring that is optionally substituted with one or more, in one embodiment one, substituent selected from F, Cl, Br, CN, CF₃, OR, carboxy, alkylenedioxy, alkylene where the alkylene group forms a fused bicyclic group with the phenyl, benzyl, thienyl or pyridyl ring, (CRR₁)ₙCO₂R, CF₂CO₂R, O(CRR₁)CO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR₁)ₙCO₂R, C₁-C₃-alkylsulfonyl, and CF₂P(═O)(OR)(OR1).

In another embodiment, G₂ is a phenyl ring that is optionally substituted with one or more, in one embodiment one, substituent selected from Cl, Br, CN, carboxy, alkylenedioxy, alkylene where the alkylene group forms a fused bicyclic group with the phenyl or thienyl ring, (CRR₁)ₙCO₂R, O(CRR₁)CO₂R, CH═CHCO₂R, C(═O)NRR1 and C₁-C₃-alkylsulfonyl.

In another embodiment, G₂ is a phenyl ring that is optionally substituted with one or more, in one embodiment one, substituent selected from Cl, CN, methoxycarbonyl, carboxy, ethoxycarbonyl, ethylenedioxy, butylene where the butylene group forms a fused bicyclic group with the phenyl or thienyl ring, OCH₂CO₂H, OCH₂CO₂Me, OCMe₂CO₂H, OCMe₂CO₂Me, CH═CHCO₂H, CH═CHCO₂Me, C(═O)NH₂, C(═O)NHMe and methanesulfonyl.

In another embodiment, G₂ is 4-methanesulfonylphenyl, 4-carboxyphenyl, 4-methoxycarbonylphenyl, 4-ethoxycarbonylphenyl, 4-cyanophenyl, 4-chlorophenyl, 4-aminocarbonylphenyl, 4-methylaminocarbonylphenyl, phenyl, 3,4-ethylenedioxyphenyl, 3-cyanophenyl, 3-thienyl, 2-thienyl, 4-(2-methoxycarbonylethenyl)phenyl, 4-(2-carboxyethenyl)phenyl, 2-(5,6,7,8-tetrahydro)naphthyl, 4-methoxycarbonyl-methoxyphenyl, 4-carboxymethoxyphenyl, 3-methoxycarbonylphenyl, 3-carboxyphenyl, 4-(1-ethoxycarbonyl-1-methyl)ethoxyphenyl or 4-(1-carboxy-1-methyl)ethoxyphenyl. In another embodiment, G₂ is 4-methanesulfonylphenyl, 4-carboxyphenyl, or 4-methoxycarbonylphenyl.

In another embodiment, G₂ is 4-methanesulfonylphenyl, 4-methoxycarbonylphenyl, 4-ethoxycarbonylphenyl, 4-cyanophenyl, 4-chlorophenyl, 4-aminocarbonylphenyl, 4-methylaminocarbonylphenyl.

In another embodiment, G₂ is 4-methanesulfonylphenyl, 4-methoxycarbonylphenyl, 4-cyanophenyl, 4-(2-methoxycarbonylethenyl)phenyl, 4-(2-carboxyethenyl)phenyl, 4-methoxycarbonylmethoxyphenyl, 4-carboxymethoxyphenyl, 4-(1-ethoxycarbonyl-1-methyl)ethoxyphenyl or 4-(1-carboxy-1-methyl)ethoxyphenyl.

In another embodiment, G₂ is 4-(1-carboxy-1-methyl)ethoxyphenyl.

In another embodiment, G₂ is H or C₁₋₃alkyl. In another embodiment, G₂ is H. In another embodiment, G₂ and G3 together with the atoms to which they are attached form an aryl ring. In another embodiment, G₂ and G₃ together with the atoms to which they are attached form a phenyl ring.

### 5. G₃

In another embodiment, G₃ is H, C₁-C₃ alkyl, C₁-C₃ alkoxy, Cl, F, Br, or a phenyl, benzyl or pyridyl ring, where the phenyl, benzyl or pyridyl ring is optionally and independently substituted with 1, 2, or 3 of the following moieties: Cl, F, Br, CN, carboxy, (CRR₁)ₙCO₂R, OCF₃, OCHF₂, C₁-C₃ alkyl, and C₁-C₃-alkylsulfonyl.

In another embodiment, G₃ is H or a phenyl ring, where the phenyl ring is optionally substituted with one of the following: Cl, F, Br, CN, carboxy, (CRR₁)ₙCO₂R, OCF₃, OCHF₂, C₁-C₃ alkyl, and C₁-C₃-alkylsulfonyl.

In another embodiment, G₃ is H. In another embodiment, G₃ is H when G₂ is other than H. In another embodiment, G₃ is a phenyl ring that is optionally substituted with carboxy or (CRR₁)ₙCO₂R. In another embodiment, G₃ is a phenyl ring that is optionally substituted with carboxy or methoxycarbonyl. In another embodiment, G3 is 4-carboxyphenyl or 4-methoxycarbonylphenyl. In another embodiment, G₃ is 4-carboxyphenyl or 4-methoxycarbonylphenyl when G₂ is H. In another embodiment, G₃ is 4-carboxyphenyl. In another embodiment, G₃ is 4-carboxyphenyl when G₂ is H.

### 6. G₄

In another embodiment, G4 is H, C₁-C₁₀alkyl, cycloalkyl, COOalkyl, cycloalkylalkyl, C₁₋₃alkylenephenyl, phenyl, C₁₋₃alkyleneheteroaryl or heteroaryl, where the C₁₋₃alkylenephenyl, phenyl, C₁₋₃alkyleneheteroaryl or heteroaryl ring moiety is optionally and independently substituted with 1, 2, or 3 of the following moieties: Cl, F, Br, CN, carboxy, methoxycarbonyl, CF₃, OCF₃, OR, OCHF₂, C₁-C₃ alkyl, C₁-C₃-alkylsulfonyl, (CRR1)ₙCO₂R, CF₂CO₂R, O(CRR1)CO₂R, CH═CHCO₂R, tetrazolyl (Tz1), NRR1, NRC(═O)OR1, NRC(═O)R1, OC(═O)NRR1, C(═O)NRR1, NRC(═O)C(═O)OR1, SO₂NRR1, S(O)ₘ(CRR1)CO₂R, SO₂NRR1, or CF₂P(═O)(OR)(OR1).

In another embodiment, G₄ is H, C₁-C₁₀alkyl, C₃₋₁₀cycloalkyl, COO—C₁₋₆alkyl, C₃₋₁₀cycloalkyl-C₁₋₃alkylene, C₁₋₃alkylenephenyl, phenyl, C₁₋₃alkyleneheteroaryl or heteroaryl, where the C₁₋₃alkylenephenyl, phenyl, C₁₋₃alkyleneheteroaryl or heteroaryl ring moiety is optionally and independently substituted with 1, 2, or 3 of the following moieties: Cl, F, Br, CN, carboxy, methoxycarbonyl, CF₃, OCF₃, OR, OCHF₂, C₁-C₃ alkyl, C₁-C₃-alkylsulfonyl, (CRR1)ₙCO₂R, CF₂CO₂R, O(CRR1)CO₂R, CH═CHCO₂R, tetrazolyl (Tz1), NRR1, NRC(═O)OR1, NRC(═O)R1, OC(═O)NRR1, C(═O)NRR1, NRC(═O)C(═O)OR1, SO₂NRR1, S(O)ₘ(CRR1)CO₂, SO₂NRR1, or CF₂P(═O)(OR)(OR1).

In another embodiment, G₄ is H, C₁-C₆alkyl, C₃₋₁₀cycloalkyl, COO—C₁₋₆alkyl, C₃₋₁₀cycloalkyl-C₁₋₃alkylene, C₁₋₃alkylenephenyl, phenyl, C₁₋₃alkylenepyridyl or pyridyl, where the C₁₋₃alkylenephenyl, phenyl, C₁₋₃alkylenepyridyl or pyridyl ring moiety is optionally and independently substituted with 1, 2, or 3 of the following moieties: Cl, F, Br, CN, carboxy, methoxycarbonyl, CF₃, OCF₃, OR, OCHF₂, C₁-C₃ alkyl, C₁-C₃-alkylsulfonyl, (CRR1)ₙCO₂R, CF₂CO₂R, O(CRR1)CO₂R, CH═CHCO₂R, tetrazolyl (Tz1), NRR1, NRC(═O)OR1, NRC(═O)R1, OC(═O)NRR1, C(═O)NRR1, NRC(═O)C(═O)OR1, SO₂NRR1, S(O)ₘ(CRR1)CO₂R, SO₂NRR1, or CF₂P(═O)(OR)(OR1).

In another embodiment, G4 is H, methyl, ethyl, propyl, cyclopropyl, COOEt, cyclopropylmethylene, benzyl, phenethyl, phenyl, CH₂-pyridyl or pyridyl, where the benzyl, phenethyl, phenyl, CH₂-pyridyl or pyridyl ring moiety is optionally and independently substituted with 1, 2, or 3 of the following moieties: Cl, F, Br, CN, carboxy, methoxycarbonyl, CF₃, OCF₃, OR, OCHF₂, C₁-C₃ alkyl, C₁-C₃-alkylsulfonyl, (CRR1)ₙCO₂R, CF₂CO₂R, O(CRR1)CO₂R, CH═CHCO₂R, tetrazolyl (Tz1), NRR1, NRC(═O)OR1, NRC(═O)R1, OC(═O)NRR1, C(═O)NRR1, NRC(═O)C(═O)OR1, SO₂NRR1, S(O)ₘ(CRR1)CO₂, SO₂NRR1, or CF₂P(═O)(OR)(OR1).

In another embodiment, G₄ is H, methyl, ethyl, propyl, cyclopropyl, COOEt, cyclopropylmethylene, benzyl, phenethyl, phenyl or CH₂-pyridyl, where the benzyl, phenethyl, phenyl or CH₂-pyridyl ring moiety is optionally and independently substituted with 1, 2, or 3 of the following moieties: Cl, F, Br, CN, carboxy, methoxycarbonyl, ethoxycarbonyl, methoxy, methanesulfonyl, methoxycarbonylmethoxy, carboxymethoxy, NH₂, NHC(═O)Me or CF₂P(═O)(OH)₂.

In another embodiment, G₄ is H, methyl, ethyl, propyl, cyclopropyl, COOEt, cyclopropylmethylene, benzyl, 4-fluorobenzyl, phenethyl, 4-cyanobenzyl, phenyl, 3-fluorobenzyl, 3,5-dimethoxybenzyl, CH₂-2-pyridyl, 3-aminobenzyl, CH₂-cyclopropyl, 3-acetamidobenzyl, 4-methanesulfonylbenzyl, 4-methoxycarbonylbenzyl, 4-carboxybenzyl, 3-methoxybenzyl, 4-ethoxycarbonylphenyl, 3-chloro-4-methoxycarbonylmethoxybenzyl, 3-chloro-4-carboxymethoxybenzyl or 3-bromo-4-difluorophosphonomethylbenzyl.

In another embodiment, G₄ is 3,5-dimethoxybenzyl, 4-carboxybenzyl, 3-chloro-4-methoxycarbonylmethoxybenzyl or 3-chloro-4-carboxymethoxybenzyl. In another embodiment, G₄ is 4-carboxybenzyl or 3-chloro-4-methoxycarbonylmethoxybenzyl. In another embodiment, G₄ is 3-chloro-4-methoxycarbonylmethoxybenzyl.

In another embodiment, G4 is 4-carboxybenzyl or 4-methanesulfonylbenzyl.

In another embodiment, G₄ is ethoxycarbonyl, benzyl, hydrogen, methyl or ethyl.

In another embodiment, G₃ and G₄ can be linked together to form an alicyclic, aromatic or aromatic ring.

In another embodiment, G₂ and G₃ can be linked together to form an alicyclic, aromatic or aromatic ring.

### 7. Further Embodiments

In one embodiment, the compounds for use in the compositions and methods provided herein have formula II:

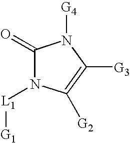

where G₁-G₄ and L₁ are as defined elsewhere herein.

In another embodiment, the compound is selected from the compounds shown in Table 1.

TABLE 1

| | | | Ac- |
|---|---|---|---|
| Com- | | | tiv- |
| pound | | | ity |
| ID | Structure | Chemical Name | Code |
| 1 | 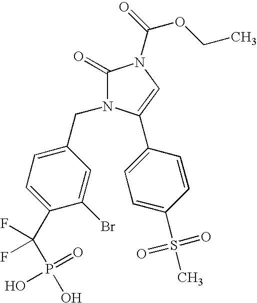 | 3-[3-Bromo-4- (difluoro- phosphono-methyl)- benzyl]-4-(4- methanesulfonyl- phenyl)-2-oxo- 2,3-dihydro-imidazole- 1-carboxylic acid ethyl ester | B |
| 2 | 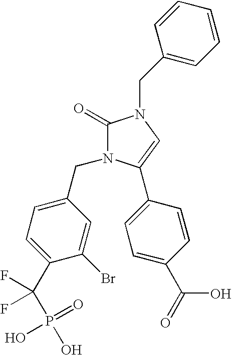 | 4-{1-Benzyl-3- [3-bromo-4- (difluoro-phosphono- methyl)- benzyl]-2-oxo- 2,3-dihydro-1H- imidazol-4-yl}- benzoic acid | C |
| 3 | 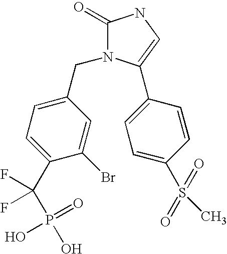 | ({2-Bromo-4- [5-(4- methanesulfonyl- phenyl)-2-oxo- 2,3-dihydro-imidazol- 1-ylmethyl]- phenyl}- difluoro-methyl)- phosphonic acid | C |
| 4 | 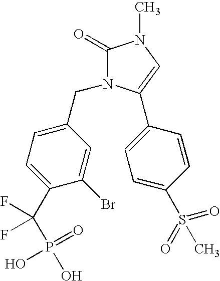 | ({2-Bromo-4- [5-(4- methanesulfonyl- phenyl)-3-methyl- 2-oxo-2,3-dihydro- imidazol-1- ylmethyl]- phenyl}-difluoro- methyl)- phosphonic acid | B |
| 6 | 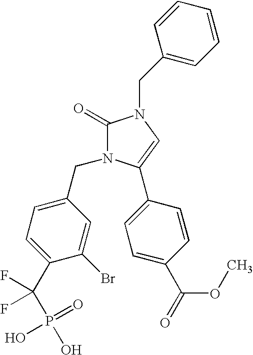 | 4-{1-Benzyl-3- [3-bromo-4- (difluoro- phosphono-methyl)- benzyl]-2- oxo-2,3-dihydro-1H- imidazol-4-yl}- benzoic acid methyl ester | B |
| 7 | 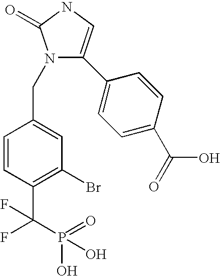 | 4-{3- [3-Bromo-4-(difluoro- phosphono- methyl)- benzyl]-2-oxo- 2,3-dihydro-1H- imidazol-4-yl}- benzoic acid | C |
| 9 | 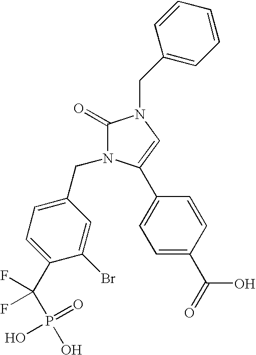 | 4-{1-Benzyl-3- [3-chloro-4- (difluoro- phosphono-methyl)- benzyl]-2-oxo-2,3- dihydro-1H- imidazol-4-yl}- benzoic acid | C |
| 10 | 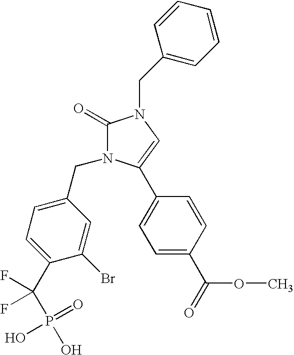 | 4-{1-Benzyl-3- [3-chloro-4- (difluoro- phosphono-methyl)- benzyl]-2-oxo-2,3- dihydro-1H- imidazol-4-yl}- benzoic acid methyl ester | C |
| 11 | 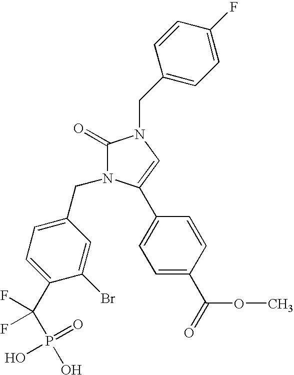 | 4-[3- [3-Bromo-4-(difluoro- phosphono- methyl)- benzyl]-1-(4- fluoro-benzyl)-2- oxo-2,3-dihydro- 1H-imidazol-4- yl]-benzoic acid methyl ester | B |
| 12 | 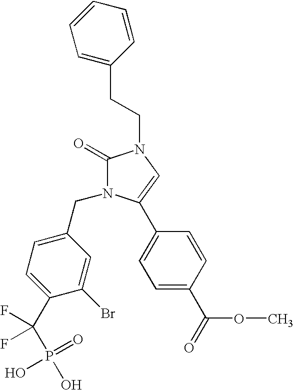 | 4-{3- [3-Bromo-4-(difluoro- phosphono- methyl)- benzyl]-2-oxo- 1-phenethyl-2,3- dihydro-1H- imidazol-4-yl}- benzoic acid methyl ester | C |
| 13 | 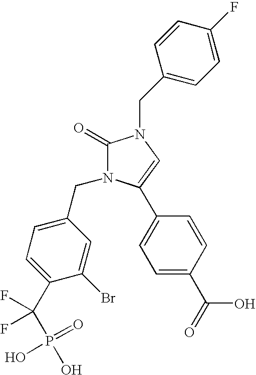 | 4-[3- [3-Bromo-4-(difluoro- phosphono- methyl)- benzyl]-1-(4- fluoro-benzyl)-2- oxo-2,3-dihydro- 1H-imidazol-4- yl]-benzoic acid | C |
| 14 | 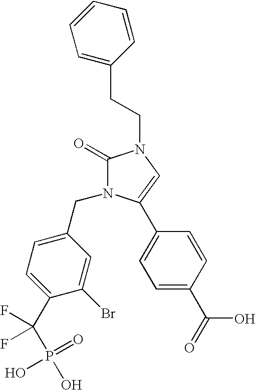 | 4-{3- [3-Bromo-4-(difluoro- phosphono- methyl)- benzyl]-2-oxo- 1-phenethyl-2,3- dihydro-1H- imidazol-4- yl}-benzoic acid | C |
| 15 | 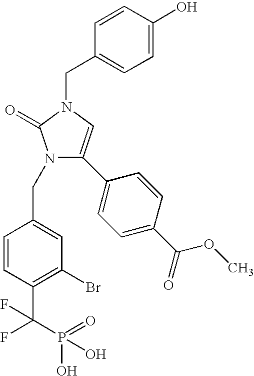 | 4-[3- [3-Bromo-4-(difluoro- phosphono- methyl)- benzyl]-1-(4- cyano-benzyl)- 2-oxo-2,3- dihydro- 1H-imidazol-4-yl]- benzoic acid methyl ester | B |
| 16 | 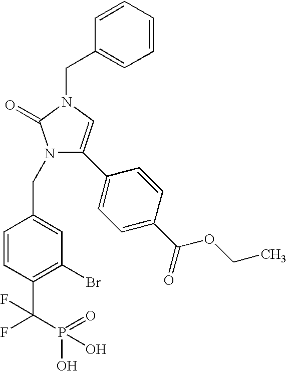 | 4-{1-Benzyl-3- [3-bromo-4- (difluoro- phosphono-methyl)- benzyl]-2-oxo-2,3- dihydro-1H- imidazol-4-yl}- benzoic acid ethyl ester | C |
| 17 | 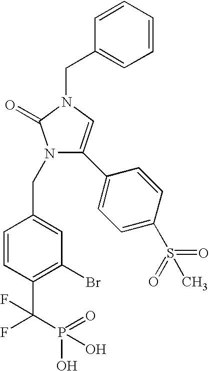 | ({4-[3-Benzyl-5-(4- methanesulfonyl- phenyl)-2-oxo- 2,3-dihydro- imidazol-1-ylmethyl]- 2-bromo-phenyl}- difluoro-methyl)- phosphonic acid | A |
| 18 | 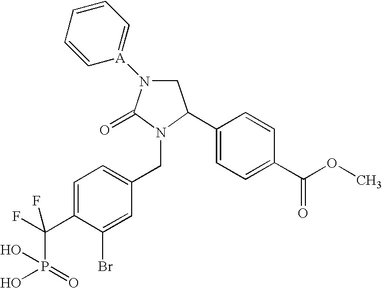 | 4-{3-[3-Bromo- 4-(difluoro- phosphono-methyl)- benzyl]-2-oxo- 1-phenyl-2,3- dihydro-1H-imidazol- 4-yl}-benzoic acid methyl ester | B |
| 19 | 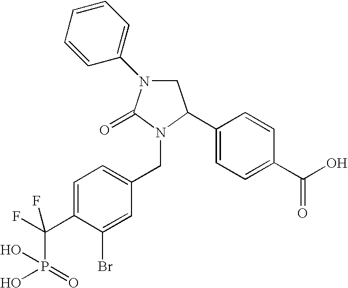 | 4-{3-[3-Bromo- 4-(difluoro- phosphono-methyl)- benzyl]-2-oxo- 1-phenyl-2,3- dihydro-1H-imidazol- 4-yl}-benzoic acid | C |
| 20 | 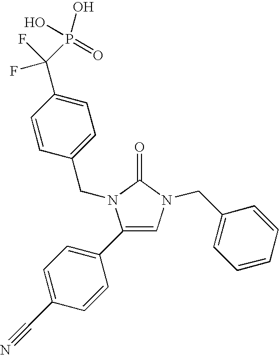 | ({4-[3-Benzyl- 5-(4-cyano-phenyl)- 2-oxo-2,3- dihydro- imidazol-1- ylmethyl]-2- bromo-phenyl}- difluoro-methyl)- phosphonic acid | B |
| 21 | 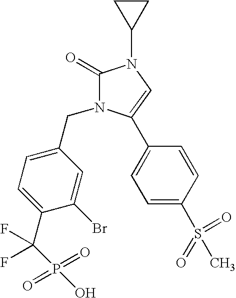 | ({2-Bromo-4-[3- cyclopropyl-5-(4- methanesulfonyl- phenyl)-2-oxo- 2,3-dihydro- imidazol-1- ylmethyl]- phenyl}- difluoro-methyl)- phosphonic acid | B |
| 22 | 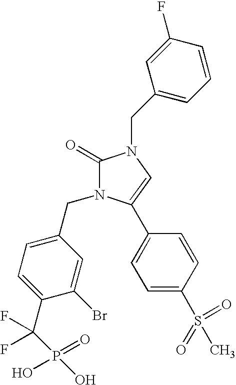 | ({2-Bromo-4-[3- (3-fluoro-benzyl)- 5-(4-methanesulfonyl- phenyl)-2- oxo-2,3- dihydro- imidazol-1- ylmethyl]- phenyl}-difluoro- methyl)- phosphonic acid | B |
| 23 | 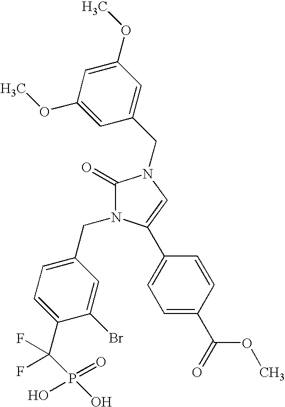 | 4-[3-[3-Bromo-4- (difluoro- phosphono-methyl)- benzyl]-1-(3,5- dimethoxy-benzyl)-2- oxo-2,3- dihydro-1H- imidazol-4-yl]-benzoic acid methyl ester | A |
| 24 | 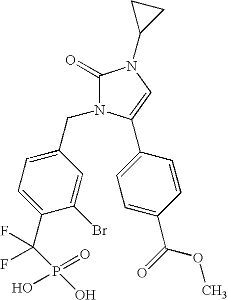 | 4-{3-[3-Bromo-4- (difluoro- phosphono-methyl)- benzyl]-1- cyclopropyl-2-oxo-2,3- dihydro-1H- imidazol-4-yl}- benzoic acid methyl ester | B |
| 25 | 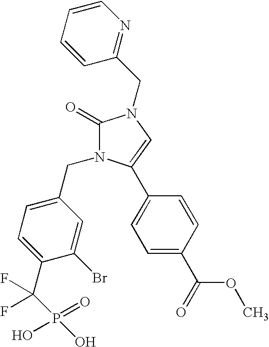 | 4-{3-[3-Bromo-4- (difluoro- phosphono-methyl)- benzyl]-2-oxo- 1-pyridin-2- ylmethyl-2,3-dihydro- 1H-imidazol-4-yl}- benzoic acid methyl ester | B |
| 26 | 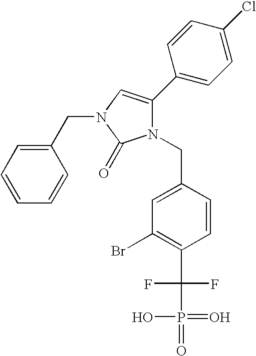 | ({4-[3-Benzyl-5- (4-chloro-phenyl)- 2-oxo-2,3- dihydro-imidazol-1- ylmethyl]- 2-bromo-phenyl}- difluoro-methyl)- phosphonic acid | B |
| 27 | 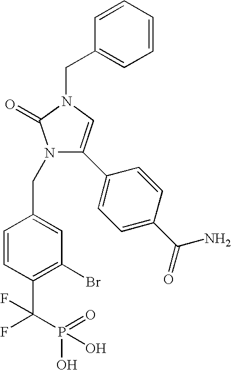 | ({4-[3-Benzyl-5- (4-carbamoyl- phenyl)-2-oxo-2,3- dihydro- imidazol-1-ylmethyl]- 2-bromo- phenyl}- difluoro-methyl)- phosphonic acid | B |
| 28 | 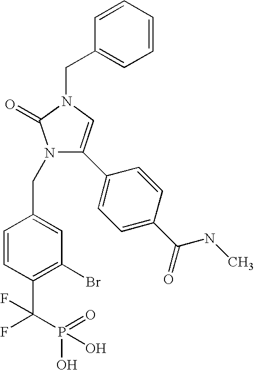 | ({4-[3-Benzyl-5- (4- methylcarbamoyl- phenyl)-2-oxo- 2,3-dihydro- imidazol-1-ylmethyl]- 2-bromo-phenyl}- difluoro-methyl)- phosphonic acid | C |
| 29 | 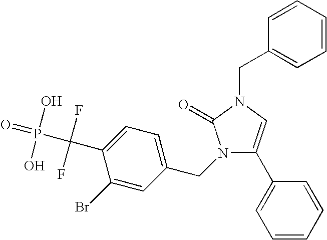 | {[4-(3-Benzyl-2- oxo-5-phenyl-2,3- dihydro-imidazol- 1-ylmethyl)-2- bromo-phenyl]- difluoro-methyl}- phosphonic acid | C |
| 30 | 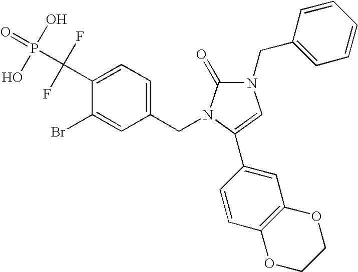 | ({4-[3-Benzyl-5- (2,3-dihydro- benzo[1,4]dioxin- 6-yl)-2-oxo-2,3- dihydro-imidazol-1- ylmethyl]-2- bromo-phenyl}- difluoro-methyl)- phosphonic acid | C |
| 31 | 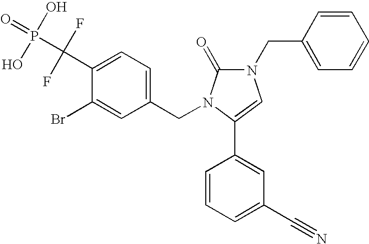 | ({4-[3-Benzyl-5- (3-cyano-phenyl)- 2-oxo-2,3-dihydro- imidazol-1- ylmethyl]-2- bromo-phenyl}- difluoro-methyl)- phosphonic acid | C |
| 32 | 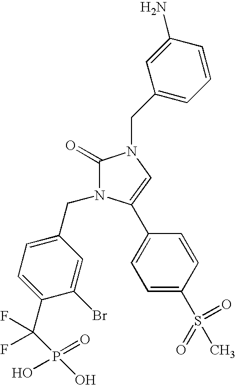 | ({4-[3-(3-Amino- benzyl)-5-(4- methanesulfonyl- phenyl)-2-oxo- 2,3-dihydro-imidazol- 1-ylmethyl]- 2-bromo-phenyl}- difluoro-methyl)- phosphonic acid | A |
| 33 | 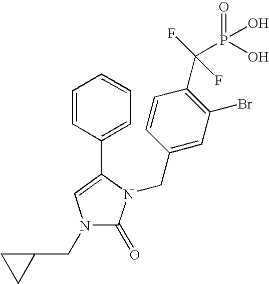 | {[2-Bromo-4-(3- cyclopropylmethyl- 2-oxo-5- phenyl-2,3- dihydro-imidazol-1- ylmethyl)- phenyl]-difluoro- methyl}- phosphonic acid | C |
| 34 | 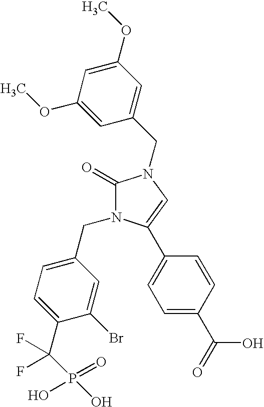 | 4-[3-[3-Bromo-4- (difluoro- phosphono-methyl)- benzyl]-1-(3,5- dimethoxy-benzyl)-2- oxo-2,3- dihydro-1H- imidazol-4-yl]-benzoic acid | C |
| 35 | 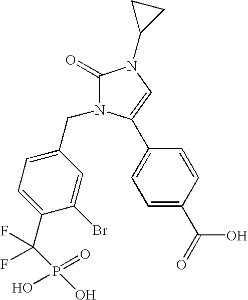 | 4-{3-[3-Bromo-4- (difluoro- phosphono-methyl)- benzyl]-1- cyclopropyl-2- oxo-2,3-dihydro-1H- imidazol-4-yl}- benzoic acid | C |
| 36 | 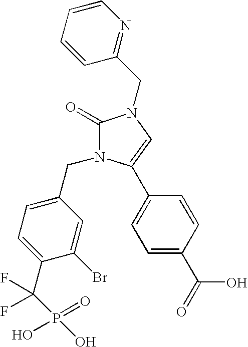 | 4-{3-[3-Bromo-4- (difluoro- phosphono-methyl)- benzyl]-2-oxo- 1-pyridin-2-ylmethyl- 2,3-dihydro- 1H-imidazol-4-yl}- benzoic acid | C |
| 37 | 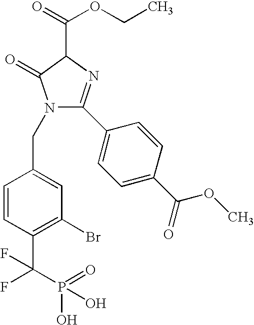 | 3-[3-Bromo-4- (difluoro- phosphono-methyl)- benzyl]-4-(4- methoxycarbonyl- phenyl)-2- oxo- 2,3-dihydro- imidazole-1-carboxylic acid ethyl ester | C |
| 38 | 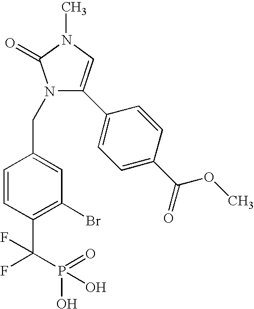 | 4-{3-[3-Bromo- 4-(difluoro- phosphono-methyl)- benzyl]-1- methyl-2-oxo-2,3- dihydro-1H- imidazol-4-yl}- benzoic acid methyl ester | C |
| 39 | 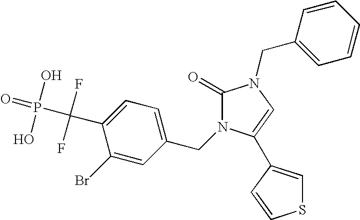 | {[4-(3-Benzyl-2- oxo-5-thiophen-3- yl-2,3-dihydro- imidazol-1- ylmethyl)-2- bromo-phenyl]- difluoro-methyl}- phosphonic acid | C |
| 40 | 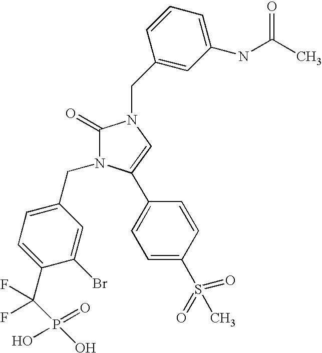 | ({4-[3-(3- Acetylamino- benzyl)-5- (4-methanesulfonyl- phenyl)-2-oxo- 2,3-dihydro-imidazol- 1-ylmethyl]- 2-bromo-phenyl}- difluoro-methyl)- phosphonic acid | A |
| 41 | 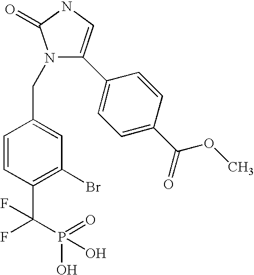 | 4-{3-[3-Bromo- 4-(difluoro- phosphono-methyl)- benzyl]-2-oxo- 2,3-dihydro-1H- imidazol-4-yl}- benzoic acid methyl ester | C |
| 42 | 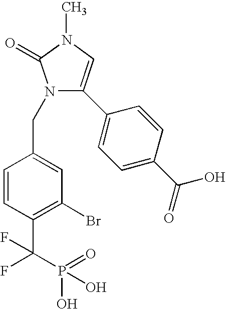 | 4-{3-[3-Bromo- 4-(difluoro- phosphono-methyl)- benzyl]-1- methyl-2-oxo-2,3- dihydro-1H- imidazol-4-yl}- benzoic acid | C |
| 43 | 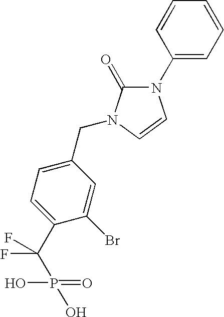 | {[2-Bromo-4- (2-oxo-3-phenyl-2,3- dihydro-imidazol- 1-ylmethyl)- phenyl]- difluoro-methyl}- phosphonic acid | D |
| 44 | 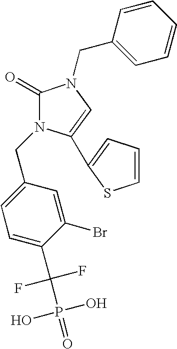 | {[4-(3-Benzyl- 2-oxo-5-thiophen-2- yl-2,3-dihydro- imidazol-1- ylmethyl)-2- bromo-phenyl]- difluoro-methyl}- phosphonic acid | C |
| 45 | 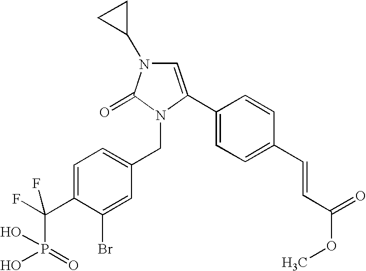 | 3-(4-{3-[3- Bromo-4-(difluoro- phosphono-methyl)- benzyl]-1- cyclopropyl-2- oxo-2,3-dihydro-1H- imidazol-4-yl}- phenyl)-acrylic acid methyl ester | A |
| 46 | 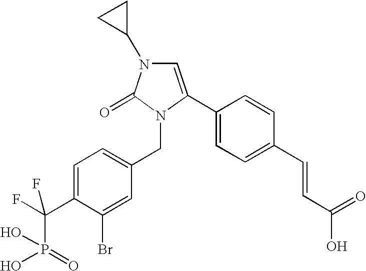 | 3-(4-{3-[3- Bromo-4-(difluoro- phosphono-methyl)- benzyl]-1- cyclopropyl-2- oxo-2,3-dihydro-1H- imidazol-4-yl}- phenyl)-acrylic acid | A |
| 47 | 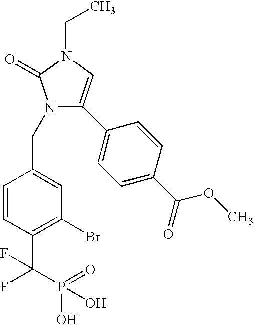 | 4-{3-[3- Bromo-4-(difluoro- phosphono-methyl)- benzyl]-1- ethyl-2-oxo-2,3- dihydro-1H- imidazol-4-yl}- benzoic acid methyl ester | B |
| 48 | 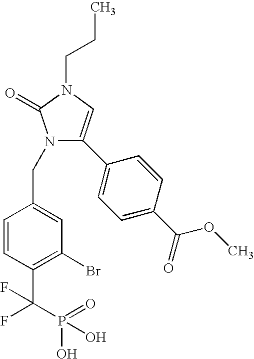 | 4-{3-[3- Bromo-4-(difluoro- phosphono-methyl)- benzyl]-2-oxo- 1-propyl-2,3- dihydro-1H-imidazol- 4-yl}- benzoic acid methyl ester | B |
| 49 | 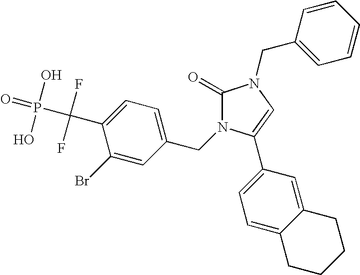 | ({4-[3-Benzyl- 2-oxo-5-(5,6,7,8- tetrahydro- naphthalen-2-yl)-2,3- dihydro- imidazol-1- ylmethyl]-2- bromo-phenyl}- difluoro-methyl)- phosphonic acid | C |
| 50 | 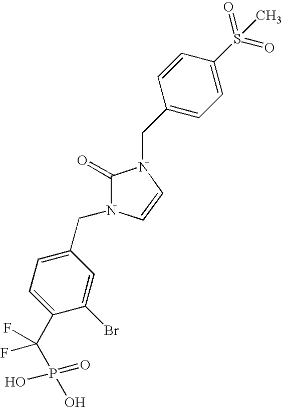 | ({2-Bromo-4-[3-(4- methanesulfonyl- benzyl)-2-oxo- 2,3-dihydro-imidazol- 1-ylmethyl]- phenyl}- difluoro-methyl)- phosphonic acid | C |
| 51 | 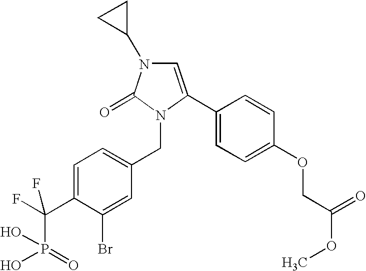 | (4-{3-[3-Bromo- 4-(difluoro- phosphono-methyl)- benzyl]-1- cyclopropyl-2-oxo- 2,3-dihydro-1H- imidazol-4-yl}- phenoxy)-acetic acid methyl ester | B |
| 52 | 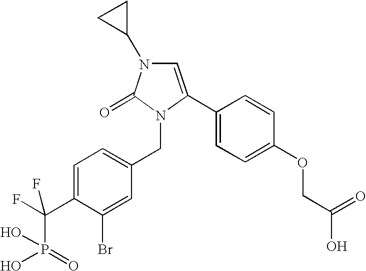 | (4-{3-[3-Bromo- 4-(difluoro- phosphono-methyl)- benzyl]-1- cyclopropyl-2-oxo- 2,3-dihydro-1H- imidazol-4-yl}- phenoxy)-acetic acid | A |
| 53 | 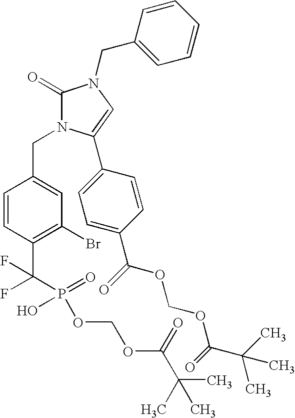 | 4-[1-Benzyl-3-(3- bromo-4-{[(2,2- dimethyl- propionyloxy- methoxy)- hydroxy-phosphoryl]- difluoro- methyl}- benzyl)-2-oxo-2,3- dihydro-1H-imidazol- 4-yl]-benzoic acid 2,2-dimethyl- propionyloxymethyl ester | D |
| 54 | 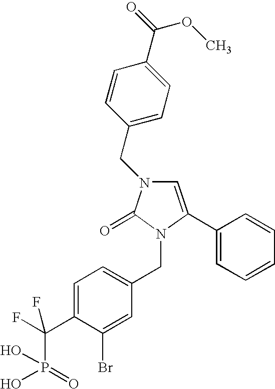 | 4-{3-[3-Bromo- 4-(difluoro- phosphono-methyl)- benzyl]-2-oxo- 4-phenyl-2,3-dihydro- imidazol-1- ylmethyl}- benzoic acid methyl ester | B |
| 55 | 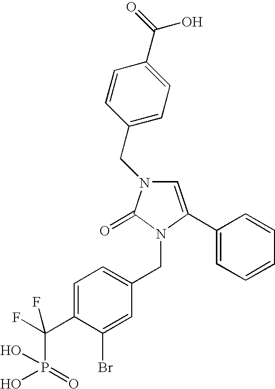 | 4-{3-[3-Bromo- 4-(difluoro- phosphono-methyl)- benzyl]-2-oxo- 4-phenyl-2,3-dihydro- imidazol-1- ylmethyl}- benzoic acid | C |
| 56 | 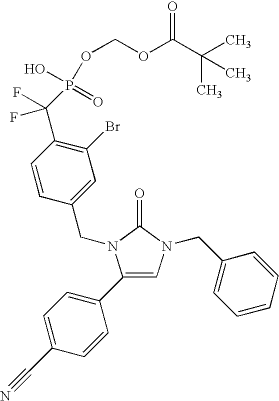 | 2,2-Dimethyl- propionic acid ({4- [3-benzyl-5- (4-cyano-phenyl)-2- oxo-2,3-dihydro- imidazol-1- ylmethyl]-2- bromo-phenyl}- difluoro- methyl)-hydroxy- phosphinoyloxymethyl ester | D |
| 57 | 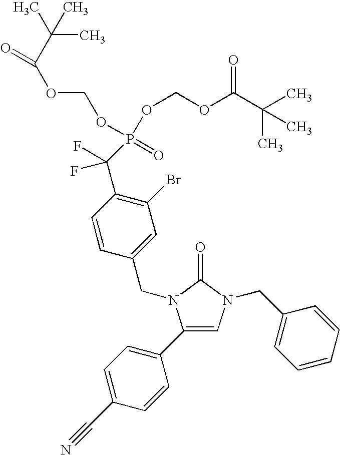 | 2,2-Dimethyl- propionic acid ({4- [3-benzyl-5-(4- cyano-phenyl)-2- oxo-2,3-dihydro- imidazol-1- ylmethyl]- 2-bromo-phenyl}- difluoro-methyl)- (2,2-dimethyl- propionyloxy- methoxy)- phosphinoyloxymethyl ester | E |
| 58 | 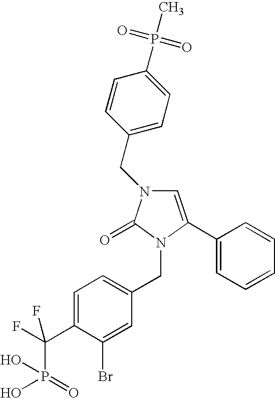 | ({2-Bromo-4-[3-(4- methanesulfonyl- benzyl)-2-oxo-5- phenyl-2,3- dihydro-imidazol-1- ylmethyl]- phenyl}-difluoro- methyl)- phosphonic acid | C |
| 59 | 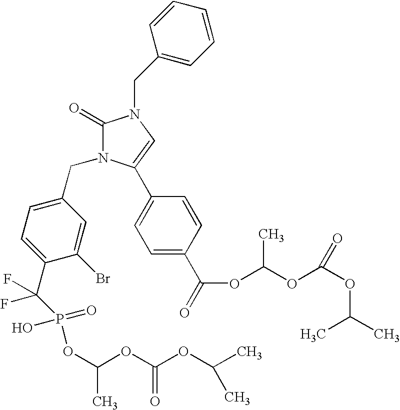 | 4-[1-Benzyl-3- (3-bromo-4- {difluoro- [hydroxy-(1- isopropoxy- carbonyloxy- ethoxy)- phosphoryl]- methyl}-benzyl)-2- oxo-2,3-dihydro- 1H-imidazol-4- yl]-benzoic acid 1- isopropoxy- carbonyloxy- ethyl ester | D |
| 60 | 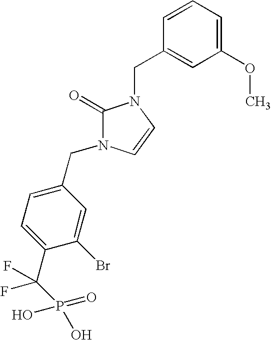 | ({2-Bromo-4- [3-(3-methoxy- benzyl)-2-oxo- 2,3-dihydro- imidazol-1- ylmethyl]- phenyl}- difluoro-methyl)- phosphonic acid | D |
| 61 | 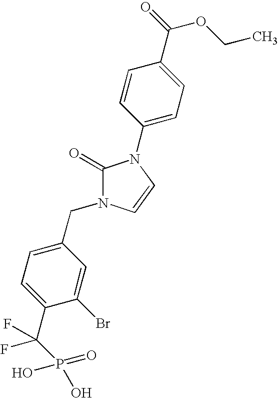 | 4-{3-[3-Bromo- 4-(difluoro- phosphono-methyl)- benzyl]-2-oxo- 2,3- dihydro-imidazol- 1-yl}- benzoic acid ethyl ester | C |
| 62 | 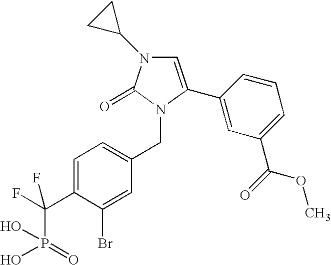 | 3-{3-[3-Bromo- 4-(difluoro- phosphono-methyl)- benzyl]-1- cyclopropyl-2- oxo-2,3-dihydro-1H- imidazol-4-yl}- benzoic acid methyl ester | C |
| 63 | 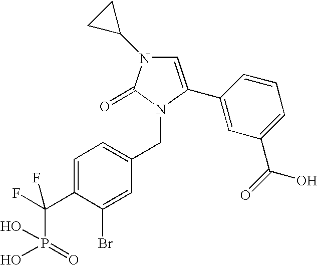 | 3-{3-[3-Bromo- 4-(difluoro- phosphono-methyl)- benzyl]-1- cyclopropyl-2- oxo-2,3-dihydro-1H- imidazol-4-yl}- benzoic acid | C |
| 64 | 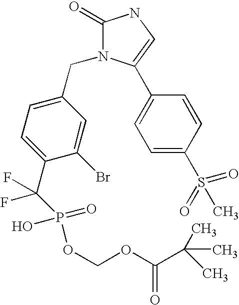 | 2,2-Dimethyl- propionic acid ({2- bromo-4-[5-(4- methanesulfonyl- phenyl)-2- oxo-2,3-dihydro- imidazol-1- ylmethyl]- phenyl}- difluoro- methyl)-hydroxy phosphinoyloxymethyl ester | D |
| 65 | 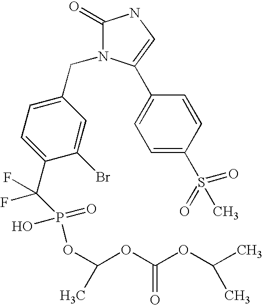 | ({2-Bromo-4-[5-(4- methanesulfonyl- phenyl)-2-oxo- 2,3-dihydro- imidazol-1-ylmethyl]phenyl}- difluoro-methyl) phosphonic acid mono-(1- isopropoxy- carbonyloxy- ethyl) ester | D |
| 66 | 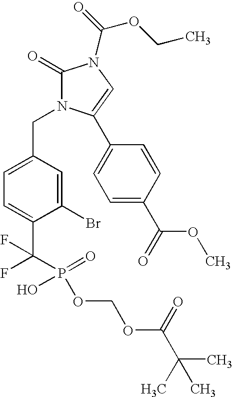 | 3-(3-Bromo-4- {[(2,2-dimethyl- propionyloxy- methoxy)- hydroxy- phosphoryl]- difluoro-methyl}- benzyl)-4-(4- methoxycarbonyl- phenyl)-2-oxo- 2,3-dihydro- imidazole-1- carboxylic acid ethyl ester | E |
| 67 | 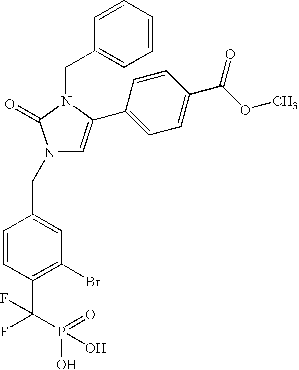 | 4-{3-Benzyl-1- [3-bromo-4- (difluoro- phosphono-methyl)- benzyl]-2-oxo-2,3- dihydro-1H- imidazol-4-yl}- benzoic acid methyl ester | C |
| 68 | 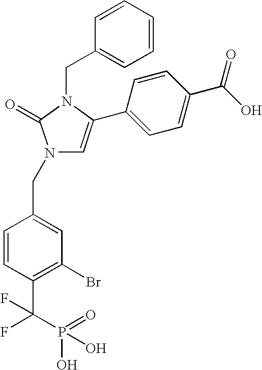 | 4-{3-Benzyl-1- [3-bromo-4- (difluoro- phosphono-methyl)- benzyl]-2-oxo-2,3- dihydro-1H- imidazol-4-yl}- benzoic acid | C |
| 69 | 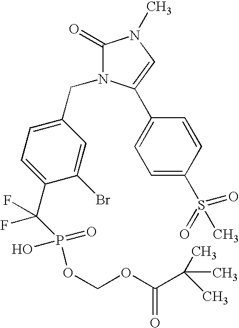 | 2,2-Dimethyl- propionic acid ({2- bromo-4-[5-(4- methanesulfonyl- phenyl)-3-methyl- 2-oxo-2,3- dihydro-imidazol- 1-ylmethyl]- phenyl}-difluoro- methyl)-hydroxy- phosphinoyloxymethyl ester | D |
| 70 | 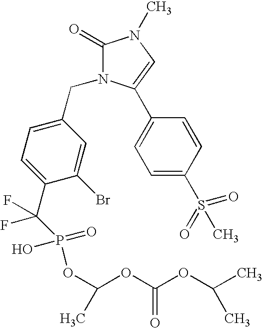 | ({2-Bromo-4-[5-(4- methanesulfonyl- phenyl)-3-methyl- 2-oxo-2,3-dihydro- imidazol-1- ylmethyl]- phenyl}-difluoro- methyl)-phosphonic acid mono-(1- isopropoxy- carbonyloxy- ethyl) ester | E |
| 71 | 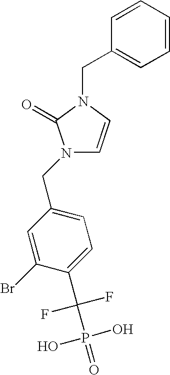 | {[4-(3-Benzyl-2- oxo-2,3 -dihydro- imidazol-1-ylmethyl)- 2-bromo- phenyl]- difluoro-methyl}- phosphonic acid | D |
| 72 | 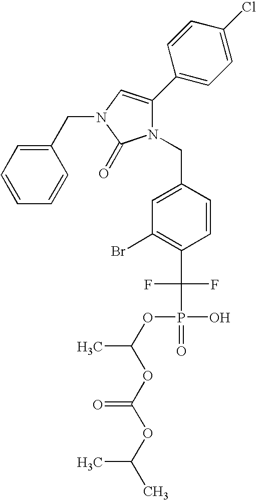 | ({4-[3-Benzyl-5- (4-chloro-phenyl)- 2-oxo-2,3-dihydro- imidazol-1- ylmethyl]-2- bromo-phenyl}- difluoro-methyl)- phosphonic acid mono-(1-isopropoxy- carbonyloxy- ethyl) ester | D |
| 73 | 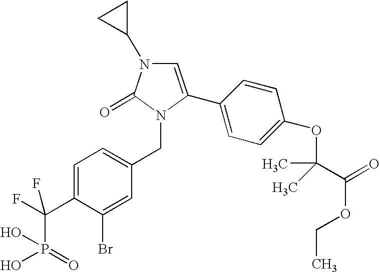 | 2-(4-{3-[3- Bromo-4-(difluoro- phosphono-methyl)- benzyl]-1- cyclopropyl-2- oxo-2,3-dihydro-1H- imidazol-4-yl}- phenoxy)-2-methyl- propionic acid ethyl ester | B |
| 74 | 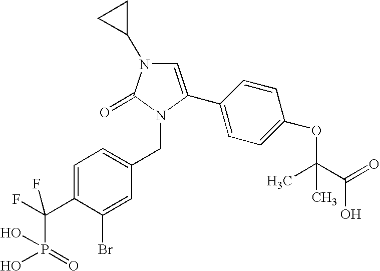 | 2-(4-{3-[3- Bromo-4-(difluoro- phosphono-methyl)- benzyl]-1- cyclopropyl-2-oxo- 2,3-dihydro-1H- imidazol-4-yl}- phenoxy)-2-methyl- propionic acid | A |
| 75 | 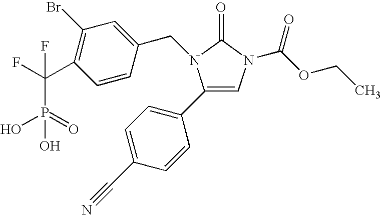 | 3-[3-Bromo-4- (difluoro- phosphono-methyl)- benzyl]-4-(4- cyano-phenyl)-2- oxo-2,3-dihydro- imidazole-1-carboxylic acid ethyl ester | B |
| 76 | 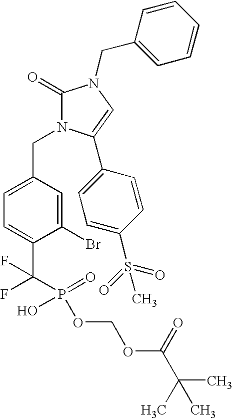 | 2,2-Dimethyl- propionic acid ({4- [3-benzyl-5-(4- methanesulfonyl- phenyl)-2- oxo-2,3-dihydro- imidazol-1-ylmethyl]- 2-bromo- phenyl}-difluoro- methyl)-hydroxy- phosphinoyloxymethyl ester | C |
| 77 | 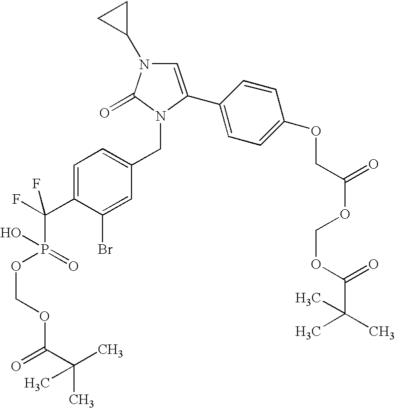 | 2,2-Dimethyl- propionic acid 2-{4- [3-(3-bromo-4- {[(2,2-dimethyl- propionyloxy- methoxy)- hydroxy- phosphoryl]- difluoro-methyl}- benzyl)-1- cyclopropyl-2-oxo-2,3- dihydro-1H- imidazol-4-yl]- phenoxy}- acetoxymethyl ester | C |
| 78 | 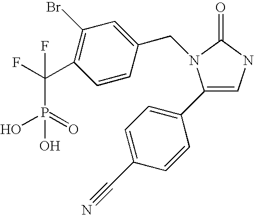 | ({2-Bromo-4- [5-(4-cyano-phenyl)- 2-oxo-2,3-dihydro- imidazol-1- ylmethyl]- phenyl}-difluoro- methyl)-phosphonic acid | B |
| 79 | 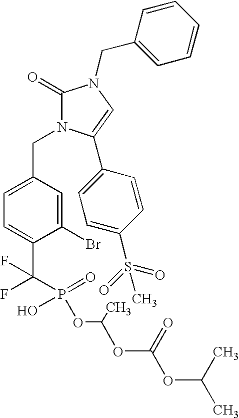 | ({4-[3- Benzyl-5-(4- methanesulfonyl- phenyl)-2-oxo- 2,3-dihydro-imidazol- 1-ylmethyl]- 2-bromo-phenyl}- difluoro-methyl)- phosphonic acid mono-(1- isopropoxy- carbonyloxy- ethyl) ester | B |
| 80 | 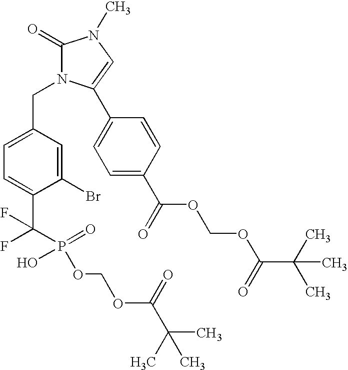 | 4-[3-(3- Bromo-4-{[(2, 2-dimethyl- propionyloxy- methoxy)-hydroxy- phosphoryl]- difluoro-methyl}- benzyl)-1-methyl- 2-oxo-2,3- dihydro-1H- imidazol- 4-yl]-benzoic acid 2,2-dimethyl- propionyloxymethyl ester | D |
| 81 | 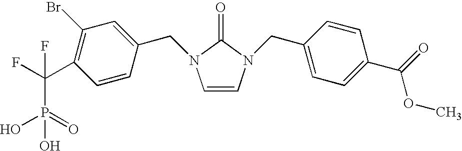 | 4-{3-[3- Bromo-4-(difluoro- phosphono-methyl)- benzyl]-2-oxo- 2,3-dihydro- imidazol-1-ylmethyl}- benzoic acid methyl ester | C |
| 82 | 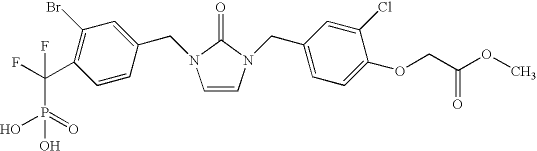 | (4-{3-[3- Bromo-4-(difluoro- phosphono-methyl)- benzyl]-2-oxo- 2,3-dihydro-imidazol- 1-ylmethyl}- 2-chloro-phenoxy)- acetic acid methyl ester | C |
| 83 | 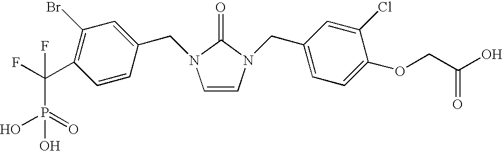 | (4-{3-[3- Bromo-4-(difluoro- phosphono-methyl)- benzyl]-2-oxo- 2,3-dihydro- imidazol-1-ylmethyl}- 2-chloro-phenoxy)- acetic acid | C |
| 84 | 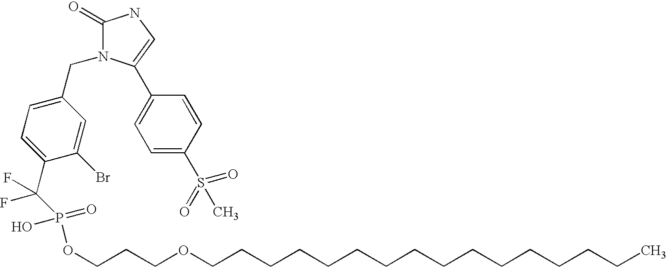 | ({2-Bromo-4-[5-(4- methanesulfonyl- phenyl)-2-oxo- 2,3-dihydro-imidazol- 1-ylmethyl]- phenyl}- difluoro-methyl)- phosphonic acid mono-(3- hexadecyloxy-propyl) ester | D |
| 85 | 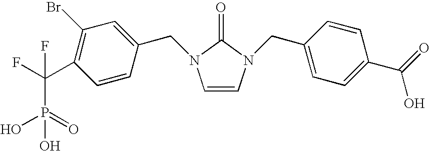 | 4-{3-[3- Bromo-4-(difluoro- phosphono-methyl)- benzyl]-2-oxo- 2,3-dihydro- imidazol-1-ylmethyl}- benzoic acid | C |
| 86 | 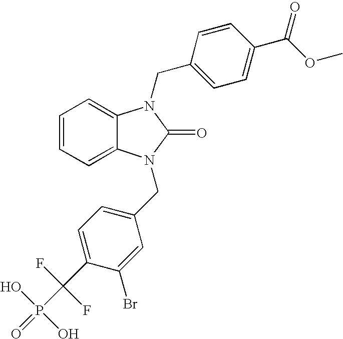 | 4-{3-[3- Bromo-4-(difluoro- phosphono-methyl)- benzyl]-2-oxo- 2,3-dihydro- benzoimidazol-1- ylmethyl}-benzoic acid methyl ester | C |
| 87 | 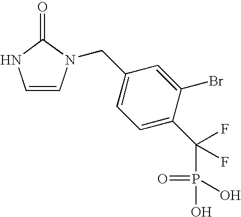 | {[2-Bromo-4-(2- oxo-2,3-dihydro- imidazol-1- ylmethyl)-phenyl]- difluoro-methyl}- phosphonic acid | D |
| 88 | 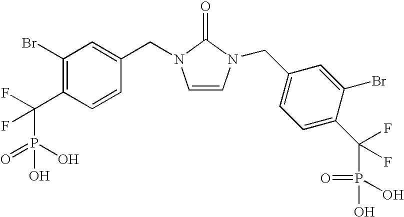 | [(2-Bromo-4-{3- [3-bromo-4- (difluoro- phosphono-methyl)- benzyl]-2- oxo-2,3-dihydro- imidazol-1- ylmethyl}-phenyl)- difluoro-methyl]- phosphonic acid | A |
| 89 | 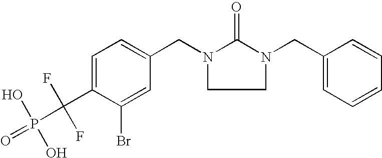 | {[4-(3- Benzyl-2-oxo- imidazolidin- 1-ylmethyl)-2- bromo-phenyl]- difluoro-methyl}- phosphonic acid | C |

Activity Class Key

| Class abbreviation | IC₅₀ range (nM) |
|---|---|
| A | <99 |
| B | 100-249 |
| C | 250-2499 |
| D | 2500-24999 |
| E | >25000 |

Mass spectral data for the compounds of Table 1 is shown in Table 2.

TABLE 2

| Compound | | | |
|---|---|---|---|
| ID | [M + H]+ | [M + NH4]+ | [M − H]− |
| 1 | 610 | | |
| 2 | | | 591 |
| 3 | | | 536 |
| 4 | | | 550 |
| 6 | | | 606 |
| 7 | | | 502 |
| 9 | | | 547 |
| 10 | | | 561 |
| 11 | | | 624 |
| 12 | | | 620 |
| 13 | | | 611 |
| 14 | | | 606 |
| 15 | 633 | | |
| 16 | 622 | | |
| 17 | 628 | | 626 |
| 18 | 593, 595 | | 591, 593 |
| 19 | 579, 581 | | 577, 579 |
| 20 | 574, 576 | 591, 593 | 572, 574 |
| 21 | | | 576 |
| 22 | | | 644 |
| 23 | 668 | | |
| 24 | 558 | | |
| 25 | 609 | | |
| 26 | 583, 585 | 602, 604 | 581, 583 |
| 27 | 593 | | |
| 28 | | | 605 |
| 29 | 549, 551 | | 547, 549 |
| 30 | 607, 609 | | 605, 607 |
| 31 | 574, 576 | 591, 593 | 572, 574 |
| 32 | | | 641 |
| 33 | 513, 515 | | 511, 513 |
| 34 | 652 | | |
| 35 | 544 | | |
| 36 | 595 | | |
| 37 | 590 | | 588 |
| 38 | | | 530 |
| 39 | 555, 557 | | 553, 555 |
| 40 | | | 683 |
| 41 | 518 | | |
| 42 | 518 | | 516 |
| 43 | 458 | | |
| 44 | 555, 557 | | 553, 555 |
| 45 | 583, 585 | | 581, 583 |
| 46 | 569, 571 | | 567, 569 |
| 47 | 546 | | |
| 48 | | | 558 |
| 49 | 603, 605 | | 601, 603 |
| 50 | | | 550 |
| 51 | 587, 589 | | 585, 587 |
| 52 | 573, 575 | | 571, 573 |
| 53 | 822 | | 820 |
| 54 | 607, 609 | 624, 626 | 605, 607 |
| 55 | 593, 595 | 610, 612 | 591, 593 |
| 56 | 688, 690 | 705, 070 | 686, 688 |
| 57 | | 819, 821 | |
| 58 | 627, 629 | 644, 646 | 625, 627 |
| 59 | | | 852 |
| 60 | 504 | | |
| 61 | 530 | | |
| 62 | 557, 559 | | 555, 557 |
| 63 | 543, 545 | | 541, 543 |
| 64 | | | 650 |
| 65 | | | 666 |
| 66 | | 720 | 702 |
| 67 | 608 | | 606 |
| 68 | 594 | | 592 |
| 69 | | | 664 |
| 70 | | | 680 |
| 71 | 473, 475 | 490, 492 | 471, 473 |
| 72 | | | 711, 713 |
| 73 | 629, 631 | | 627, 629 |
| 74 | 601, 603 | | 599, 601 |
| 75 | 556, 558 | 573, 575 | 554, 556 |
| 76 | 742 | | 740 |
| 77 | 801, 803 | | 799, 801 |
| 78 | 484, 486 | 501, 503 | 482, 484 |
| 79 | | | 757 |
| 80 | 747 | | 745 |
| 81 | 531, 533 | 548, 550 | 529, 531 |
| 82 | 595, 597, 599 | 612, 614, 616 | 593, 595, 597 |
| 83 | 581, 583, 585 | 598, 600, 602 | 579, 581, 583 |
| 84 | | | 819 |
| 85 | 517, 519 | 534, 536 | 515, 517 |
| 86 | 581, 583 | 598, 600 | 579, 581 |
| 87 | 383, 385 | 400, 402 | 381, 383 |
| 88 | 681, 683, 685 | 698, 700, 702 | 679, 681, 683 |
| 89 | 475, 477 | 492, 494 | 473, 475 |

### C. Preparation of the Compounds

The compounds for use in the compositions and methods provided herein may be obtained from commercial sources (e.g., Aldrich Chemical Company, Milwaukee, Wis.), may be prepared by methods well known to those of skill in the art, or by the methods shown herein (see, the EXAMPLES). One of skill in the art would be able to prepare all of the compounds for use herein by routine modification of these methods using the appropriate starting materials. See, e.g., International Patent Application Publication No. WO 03/032916.

### D. Formulation of Pharmaceutical Compositions

The pharmaceutical compositions provided herein contain therapeutically effective amounts of one or more of the compounds provided herein that are useful in the prevention, treatment, or amelioration of one or more of the symptoms of diseases or disorders associated with protein tyrosine phosphatase, including PTP-1B, activity, or in which protein tyrosine phosphatase, including PTP-1B, activity is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with protein tyrosine phosphatase, including PTP-1B, activity or in which protein tyrosine phosphatase, including PTP-1B, activity is implicated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in vitro and in vivo systems well known to those of skill in the art and described herein (see, e.g., EXAMPLES 41 and 42) and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with protein tyrosine phosphatase, including PTP-1B, activity or in which protein tyrosine phosphatase, including PTP-1B, activity is implicated, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

### 1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

### a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Patent Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

### 2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEENâ 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### 3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

### 4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### 5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

### 6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### 7. Articles of Manufacture

The compounds or pharmaceutically acceptable derivatives may be packaged as articles of manufacture containing packaging material, a compound or pharmaceutically acceptable derivative thereof provided herein, which is effective for modulating protein tyrosine phosphatase, including PTP-1B, activity, or for treatment, prevention or amelioration of one or more symptoms of diseases or disorders in which protein tyrosine phosphatase, including PTP-1B, activity, is implicated, within the packaging material, and a label that indicates that the compound or composition, or pharmaceutically acceptable derivative thereof, is used for modulating protein tyrosine phosphatase, including PTP-1B, activity, or for treatment, prevention or amelioration of one or more symptoms of diseases or disorders in which protein tyrosine phosphatase, including PTP-1B, activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which protein tyrosine phosphatase, including PTP-1B, activity is implicated as a mediator or contributor to the symptoms or cause.

### 8. Prodrugs

One form of prodrug is to prepare acetoxymethyl esters of the compounds provided herein, which may be prepared by the general procedure reported by C. Schultz et al., J. Biol. Chem. 1993, 268:6316-6322:

A carboxylic acid (1 eq) is suspended in dry acetonitrile (2 mL/0.1 mmol). Diisopropyl amine (3.0 eq) is added followed by bromomethyl acetate (1.5 eq). The mixture is stirred under nitrogen overnight at room temperature. Acetonitrile is removed under reduced pressure to yield an oil, which is diluted in ethylacetate and washed with water (3×). The organic layer is dried over anhydrous magnesium sulfate. Filtration, followed by solvent removal under reduced pressure, affords a crude oil. The product is purified by column chromatography on silica gel, using an appropriate solvent system.

Other prodrugs can routinely be prepared from compounds provided herein by the procedures outlined in the following reports:
Stankovic C J et al., “The Role of 4-Phosphonodifluoromethyl- and 4-Phosphono-phenylalanine in the Selectivity and Cellular Uptake of SH2 Domain Ligands.” Bioorg. Med. Chem. Lett. 1997; 7(14):1909-14.
Ortmann R et al., “Acyloxyalkyl ester prodrugs of FR900098 with improved in vivo anti-malarial activity.” Bioorg. Med. Chem. Lett. 2003; 13(13):2163-6.
Hughes W T et al., “Single-dose pharmacokinetics and safety of the oral antiviral compound adefovir dipivoxil in children infected with human immunodeficiency virus type 1.” Antimicrob Agents Chemother. 2000; 44(4):1041-6.
Starrett J E Jr et al., “Synthesis and in vitro evaluation of a phosphonate prodrug: bis(pivaloyloxymethyl) 9-(2-phosphonylmethoxyethyl)adenine.” Antiviral Res. 1992; 19(3):267-73.

Such prodrug preparations are routinely prepared, once a novel drug compound is identified, such as the novel PTP-1B inhibitors disclosed herein.

Other prodrugs of the compounds provided herein are prodrugs of difluoromethylphosphonic acids and have the formulae ArCF₂P(O)(OH)(OCH(H/Me)OC(═O)OiPr, ArCF₂P(O)[(OCH(H/Me)OC(═O)OiPr]₂, ArCF₂P(O)(OH)(OCH(H/Me)OC(═O) tBu, or ArCF2P(O)[(OCH(H/Me)OC(═O)tBu]₂. Other prodrugs of the compounds provided herein have the formulae ROCH₂CHR′CH₂O—P(O)(OH)CF₂Ar or (ROCH₂CHR′CH₂O)₂—P(O)CF₂Ar, where R is C₁₄₋₂₀-n-alkyl and R′ is H, OH or OMe. Further prodrugs of the compounds provided herein are prodrugs as described in EP 0 350 287; EP 0 674 646; U.S. Pat. No. 6,599,887; U.S. Pat. No. 6,448,392; U.S. Pat. No. 6,752,981; U.S. Pat. No. 6,312,662; U.S. 2002/0173490; Friis et al. Eur. J. Pharm. Sci. 4:49-59 (1996); Erion et al. J. Am. Chem. Soc. 126:5154-5163 (2004); WO 03/095665; Krise et al. Adv. Drug. Deliv. Rev. 19:287-310 (1996); and Ettmayer et al. J. Med. Chem. 47:2393-2404 (2004). The disclosures of these patents and publications are incorporated by reference herein in their entirety.

### E. Evaluation of the Activity of the Compounds

The activity of the compounds as modulators of protein tyrosine phosphatase, including PTP-1B, may be measured in standard assays (see, e.g., Examples 41 and 42). Briefly, the assay described herein employs human recombinant PTP-1B and a pNPP substrate in a rescue assay.

### F. Methods of Use of the Compounds and Compositions

Methods of modulating the activity of a protein tyrosine phosphatase, including PTP-1B, by contacting the protein tyrosine phosphatase with a compound or composition provided herein are provided. In one embodiment, the PTP, including PTP-1B, is inhibited by the compound or composition.

Methods of increasing insulin sensitivity by administering a compound or composition provided herein are provided.

Methods of treating, preventing, or ameliorating one or more symptoms of a protein tyrosine phosphatase mediated disease, including PTP-1B mediated diseases, by administering a compound or composition provided herein are provided. Such diseases include, but are not limited to, diabetes including Type 1 and Type 2 diabetes (and associated complications such as hypertension, ischemic diseases of the large and small blood vessels, blindness, circulatory problems, kidney failure and atherosclerosis), syndrome X, metabolic syndrome, glucose intolerance, insulin resistance, leptin resistance, obesity, cancer, neurodegenerative diseases, and other diseases in which the activity of a tyrosine phosphatase or multiple tyrosine phosphatases contributes to the symptoms or pathology thereof.

### G. Combination Therapy

The compounds and compositions provided herein may also be used in combination with other active ingredients. In another embodiment, the compounds may be administered in combination, or sequentially, with another therapeutic agent. Such other therapeutic agents include those known for treatment, prevention, or amelioration of one or more symptoms of protein tyrosine phosphatase, including PTP-1B, mediated diseases. Such therapeutic agents include, but are not limited to, antiobesity agents, antidiabetics, antihypertensive agents, agents for the treatment and/or prevention of complications resulting from or associated with diabetes and agents for the treatment and/or prevention of complications and disorders resulting from or associated with obesity.

In another embodiment, the compounds provided herein may be administered in combination with one or more antiobesity agents or appetite regulating agents. Such agents include, but are not limited to, CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators or TR B agonists.

In one embodiment the antiobesity agent is leptin. In other embodiments, the antiobesity agent is dexamphetamine or amphetamine, fenfluramine or dexfenfluramine, sibutramine, orlistat, mazindol or phentermine.

In another embodiment, the antidiabetic is insulin, GLP-1 (glucagons like peptide-1) derivatives such as those disclosed in WO 98/08871, which is incorporated herein by reference, as well as orally active hypoglycemic agents. The orally active hypoglycemic agents include, but are not limited to, sulfonylureas, biguanides, meglitinides, oxadiazolidinediones, thizolidinediones, glucosidase inhibitors, glucagons antagonists such as those disclosed in WO 99/01423, GLP-1 agonists, potassium channel openers such as those disclosed in WO 98/26265 and WO 99/03861, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism such as antihyperlipidemic agents and antilipedimic agents as HMG CoA inhibitors (statins), compounds lowering food intake, PPAR and RXR agonists and agents acting on the ATP-dependent potassium channel of the B-cells.

In one embodiment the present compounds are administered in combination with insulin. In further embodiments, the present compounds are administered in combination with a sulphonylurea e.g., tolbutamide, glibenclamide, glipizide or glicazide, a biguanide e.g. metformin, a meglitinide e.g., repaglinide, a thizolidinedione e.g., troglitazone, ciglitazone, pioglitazone, rosiglitazone or compounds disclosed in WO 97/41097 such as 5-[[4-[3-Methyl-4-oxo-3,4-dihydro-2-quinazolinyl]methoxy]phenyl-methyl]thiazolidine-2,4-dione or a pharmaceutically acceptable salt thereof.

In another embodiment, the present compounds may be administered in combination with the insulin sensitizers disclosed in WO 99/19313 such as (−) 3-[4-[2-phenoxazin-10-yl)ethoxy]phenyl]-2-ethoxypropanoic acid or a pharmaceutically acceptable salts thereof, preferably the arginine salt.

In further embodiments, the present compounds are administered in combination with an α-glucosidase inhibitor e.g. miglitol or acarbose, an agent acting on the ATP-dependent potassium channel of the B-cells e.g. tolbutamide, glibenclamide, glipizide, glicazide or repaglinide, nateglinide, an antihyperlipidemic agent or antilipidemic agent e.g., cholestyramine, colestipol, clofibrate, gemfibrozil, lovastatin, pravastatin, simvastatin, probucol or dextrothyroxine,

In still further embodiments, the present compounds are administered in combination with more than one of the above-mentioned compounds e.g., in combination with a sulphonylurea and metformin, a sulphonylurea and acarbose, repaglinide and metformin, insulin and a sulphonylurea, insulin and metformin, insulin and lovastatin, CART agonist and a CCK agonist, etc.

In another embodiment, the present compounds may be administered in combination with one or more antihypertensive agents. Examples of antihypertensive agents are B-blockers such as alprenolol, atenolol, timolot, pindolol, propranolol and metoprolol, ACE (angiotensin converting enzyme) inhibitors such as benazepril, captopril, analapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, Pa., 1995.

It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients.

The following examples are provided for illustrative purposes only and are not intended to limit the scope of the invention.

### EXAMPLE 1

### 4-(2-Bromo-acetyl)-benzoic acid methyl ester (A)

To a solution of methyl 4-acetylbenzoate (25 g, 0.14 mol) in 500 mL chloroform was added, drop-wise, bromine (22.42 g, 0.14 mol) in 60 mL chloroform over a period of 3 h. The mixture was stirred over night. Water (200 mL) was carefully added to the reaction mixture, mixed well and the organic layer isolated was washed with saturated sodium hydrogen carbonate in water (200 mL), followed by brine (200 mL), dried over anhydrous sodium sulfate, filtered and filtrate evaporated to get pure product as a light yellow powder (32 g, 89%).

### 4-(1-Benzyl-2-oxo-2,3-dihydro-1H-imidazol-4-yl)-benzoic acid methyl ester (B)

To an ice cooled mixture of benzylamine (4.4 g, 41 mmol) and triethylamine (4.5 g, 45 mmol) in 200 mL chloroform was added, drop-wise, a solution of A (10.6 g, 41 mmol) in 50 mL chloroform. After allowing the mixture to warm to room temperature, it was diluted with 100 mL dichloromethane and the mixture was washed with water (2×150 mL), dried over anhydrous sodium sulfate, filtered and evaporated. The residue was dissolved in 150 mL methanol, cooled to 0° C., 40 mL glacial acetic acid was added followed by potassium cyanate (3.6 g, 45 mmol). The mixture was refluxed for 1.5 and cooled. The precipitate formed was isolated to get 3.15 g (25%) of pure B.

### Diethyl (bromodifluoromethyl)phosphonate (C)

A solution of triethyl phosphite (95.50 g, 575 mmol) in 300 mL of anhydrous diethyl ether was cooled, under a nitrogen atmosphere, to 4° C. before addition of dibromodifluoromethane (144.72 g, 690 mmol). The mixture was allowed to warm to room temperature and stirred overnight, then heated to reflux for 24 hours. Ether was removed by rotary evaporation, and the resultant liquid was distilled to afford 142.75 g (93%) of a clear colorless liquid: bp 144-145° C. (25 mmHg) [lit. bp 97-98° C. (19 mmHg) (This material is commercially available from Lancaster and Aldrich).

### [(2-Bromo-4-methyl-phenyl)-difluoromethyl]-phosphonic acid diethyl ester (D)

To activated zinc (12 g, 190 mmol) in DMA (70 mL) was added bromodifluoromethyldiethyl-phosphonate (50 g, 190 mmol) in DMA (70 mL). The resulting mixture was stirred at 45° C. for 3 hours, after which copper (I) bromide (27 g, 190 mmol) was added and stirring was continued for 0.5 hours at room temperature. 3-Bromo-4-iodotoluene (28 g, 94 mmol) was then added and the mixture was sonicated at room temperature for 12 hours. The reaction mixture was partitioned between ether and H₂O, filtered through Celite, and the organic layer was dried over MgSO₄ and concentrated in vacuo to yield 21 g (63%) of a clear, colorless oil.

### [(2-Bromo-4-bromomethyl-phenyl)-difluoro-methyl]-phosphonic acid diethyl ester (E)

To [(2-Bromo-4-methyl-phenyl)-difluoromethyl]-phosphonic acid diethyl ester (21 g, 58 mmol) in benzene (500 mL) was added N-bromosuccinimide (12 g, 68 mmol) and AIBN (0.50 g). The resulting mixture was stirred for 12 hours at room temperature in front of a 100 W bulb. It was then washed with H₂O, sat. NaHCO₃, and brine, and the organic layer was dried over MgSO4, and concentrated in vacuo. The resulting material was purified via column chromatography (4/1 hexanes/ethyl acetate) to yield 1.7 g (66%) of clear, colorless oil.

### 4-(1-Benzyl-3-{3-bromo-4-[(diethoxy-phosphoryl)-difluoro-methyl]-benzyl}-2-oxo-2,3-dihydro-1H-imidazol-4-yl)-benzoic acid methyl ester (F)

To a suspension of sodium hydride (95%, 303 mg, 12 mmol) in anhydrous dimethylformamide (30 mL) was added B (3.08 g, 10 mmol) and stirred for 15 minutes. To this solution was added E (3.45 g, 12 mmol) in 6 mL anhydrous dimethylformamide slowly. The mixture was stirred at room temperature for 16 h. Solvent was removed under reduced pressure and the residue dissolved in 100 mL ethyl acetate was washed with water (2×50 mL), dried over anhydrous sodium sulfate, filtered and evaporated. The residue obtained was loaded on a silica gel column and purified using hexane and 1:1 hexane:ethyl acetate to elute the impurities followed by 2:1 ethyl acetate:hexane to elute the compound. Fractions collected were pooled and evaporated to obtain 3.4 g (51%) of pure product.

### 4-{1-Benzyl-3-[3-bromo-4-(difluoro-phosphono-methyl)-benzyl]-2-oxo-2,3-dihydro-1H-imidazol-4-yl}-benzoic acid (G)

To F (3.4 g, 5.13 mmol) in CH₂Cl₂ (50 mL) was added bistrimethylsilyltrifluoroacetamide (13.18 g, 51.3 mmol) and reaction mixture was stirred at room temperature for 1 h after which the reaction mixture is cooled to −20° C. and iodotrimethylsilane (10.26 g, 51.3 mmol) was added drop wise. The resulting mixture was stirred at room temperature for 1.5 hours, after which it was concentrated in vacuo. The residue was dissolved in 25 mL dichloromethane and cooled in ice-bath. To this solution was added methanol (25 mL) slowly. The mixture was stirred at room temperature for 2 h and then solvent was removed under reduced pressure. The residue was dissolved in 5 mL methanol and to this was added 150 mL ethyl acetate and washed with freshly acidified (pH<1) 2.5% solution (40 mL) of sodium dithionite in water followed by brine (40 mL). The organic solution was dried over anhydrous sodium sulfate, filtered and evaporated under reduced pressure to obtain 3.11 g of product.

The above product was dissolved in 40 mL of 1:1 v/v mixture of methanol:tetrahydrofuran and to this solution was added 2.5 M aqueous sodium hydroxide solution (10 mL). After 16 hours of stirring at room temperature, solvent was removed under reduced pressure and the residue was dissolved in 25 mL water and acidified with 2.5 N HCl in water. The precipitate formed was filtered, washed with water and dried under vacuum to get 2.5 g (82%). M/z (LCMS) 590.9 (M−1), purity by LCMS: 100.00%. ¹H NMR (DMSO-d₆) δ 4.87 (s, 2H), 5.01 (s, 2H), 7.08-7.90 (m, 13H).

### EXAMPLE 2

### 4-{3-[3-Bromo-4-(difluoro-phosphono-methyl)-benzyl]-2-oxo-2,3-dihydro-1H-imidazol-4-yl}-benzoic acid

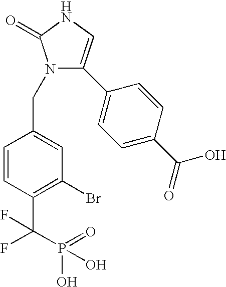

3-[3-Bromo-4-(difluoro-phosphono-methyl)-benzyl]-4-(4-methoxycarbonylphenyl)-2-oxo-2,3-dihydro-imidazole-1-carboxylic acid ethyl ester, (EXAMPLE 3) (50 mg, 0.42 mmol) was dissolved in 2 mL 0.2 M NaOH. The solution was stirred at 25° C. for 4 h then acidified with 6 M HCl to pH≈1 resulting in a fine white precipitate. The suspension was extracted thrice with 25% iPrOH/CH₂Cl₂. The combined organic extracts were dried (MgSO₄) then concentrated in vacuo. The resulting residue was triturated with ether to give the product as an off-white solid, 30 mg (70%): ¹H NMR (DMSO-d₆, 500 MHz): δ (ppm) 4.96 (s, 2H), 6.90 (d, J=2.5 Hz, 1H), 7.12 (d, J=8.0 Hz, 1H), 7.36 (s, 1H), 7.43 (dd, J=7.0 Hz, 1.5 Hz, 2H), 7.54 (d, J=7.5 Hz, 1H), 7.89 (dd, J=7.0 Hz, 1.5 Hz, 2H), 10.66 (d, J=2.5 Hz, 1H), 12.5 (bs, 3H); MS (ESI): m/z 504 (M+H)⁺.

### EXAMPLE 3

### 3-[3-Bromo-4-(difluoro-phosphono-methyl)-benzyl]-4-(4-methoxycarbonylphenyl)-2-oxo-2,3-dihydro-imidazole-1-carboxylic acid ethyl ester

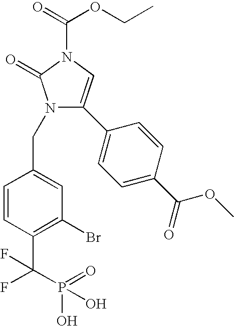

3-{3-Bromo-4-[(diethoxy-phosphoryl)-difluoro-methyl]-benzyl}-4-(4-methoxycarbonyl-phenyl)-2-oxo-2,3-dihydro-imidazole-1-carboxylic acid ethyl ester (400 mg, 0.62 mmol) was deprotected using iodotrimethylsilane as described in EXAMPLE 1 to give the title compound as a pale yellow solid, 325 mg (89%): MS (ESI): m/z 590 (M+H)⁺.

### EXAMPLE 4

### 4-{3-[3-Bromo-4-(difluoro-phosphono-methyl)-benzyl]-1-methyl-2-oxo-2,3-dihydro-1H-imidazol-4-yl}-benzoic acid methyl ester

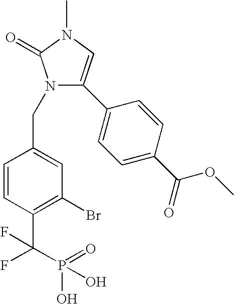

4-(3-{3-Bromo-4-[(diethoxy-phosphoryl)-difluoro-methyl]-benzyl}-1-methyl-2-oxo-2,3-dihydro-1H-imidazol-4-yl)-benzoic acid methyl ester (33 mg, 0.056 mmol) was deprotected using iodotrimethylsilane as described in EXAMPLE 1 to give the title compound as an off-white solid, 23 mg (77%): MS (ESI): m/z 532 (M+H)⁺.

### EXAMPLE 5

### 4-{3-[3-Bromo-4-(difluoro-phosphono-methyl)-benzyl]-1-methyl-2-oxo-2,3-dihydro-1H-imidazol-4-yl}-benzoic acid

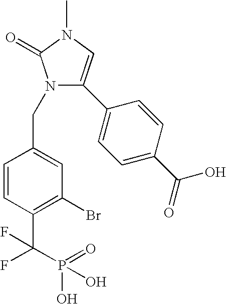

The title compound was prepared by hydrolysis of the methyl ester as described in EXAMPLE 2. 12 mg (82%): MS (ESI): m/z 518 (M+H)⁺.

### EXAMPLE 6

### 4-{3-[3-Bromo-4-(difluoro-phosphono-methyl)-benzyl]-2-oxo-2,3-dihydro-1H-imidazol-4-yl}-benzoic acid methyl ester

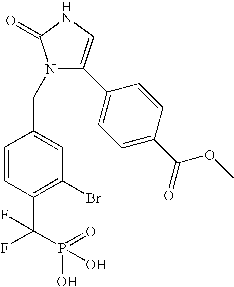

3-[3-Bromo-4-(difluoro-phosphono-methyl)-benzyl]-4-(4-methoxycarbonylphenyl)-2-oxo-2,3-dihydro-imidazole-1-carboxylic acid ethyl ester, (EXAMPLE 3) (50 mg, 0.085 mmol) was dissolved in 4 mL 2.0 M diethylamine in THF. A precipitate appeared immediately. The suspension was stirred at 25° C. for 2.5 h then the reaction mixture concentrated in vacuo. The resulting residue was triturated with dichloromethane then ether to give an off-white solid which was further purified by reversed phase C18-silica chromatography (eluting with 0 to 30% MeCN/H₂O) to give the title compound as the hygroscopic bis-dimethylamino salt, 30 mg (58%): MS (ESI): m/z 518 (M+H)⁺.

### EXAMPLE 7

### 4-{3-[3-Bromo-4-(difluoro-phosphono-methyl)-benzyl]-1-ethyl-2-oxo-2,3-dihydro-1H-imidazol-4-yl}-benzoic acid methyl ester

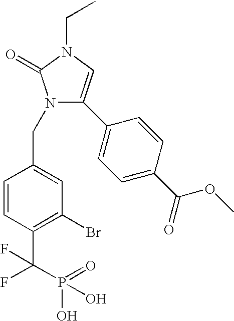

The title compound was prepared as described in EXAMPLE 4. 12 mg (66%) white solid: MS (ESI): m/z 546 (M+H)⁺.

### EXAMPLE 8

### 4-{3-[3-Bromo-4-(difluoro-phosphono-methyl)-benzyl]-2-oxo-1-propyl-2,3-dihydro-1H-imidazol-4-yl}-benzoic acid methyl ester

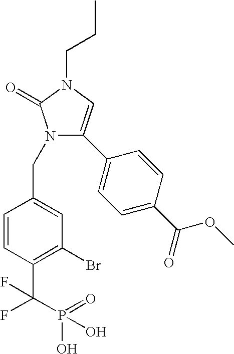

The title compound was prepared as described in EXAMPLE 4. 12 mg (57%) off-white solid: MS (ESI): m/z 558 (M−H)⁻.

### EXAMPLE 9

### 4-[3-[3-Bromo-4-(difluoro-phosphono-methyl)-benzyl]-1-(4-cyano-benzyl)-2-oxo-2,3-dihydro-1H-imidazol-4-yl]-benzoic acid methyl ester

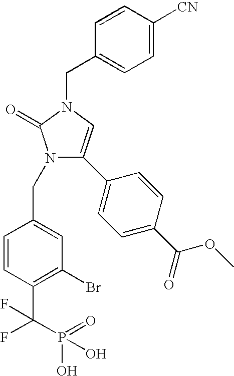

The phosphonate diethyl ester was deprotected using iodotrimethylsilane as described in EXAMPLE 1 giving the title compound as a white solid, 50 mg (68%): ¹H NMR (DMSO-d₆, 500 MHz): δ (ppm) 3.84 (s, 3H), 4.99 (s, 2H), 5.04 (s, 2H), 7.14 (d, J=8.5 Hz, 1H), 7.18 (s, 1H), 7.36 (s, 1H), 7.50 (m, 5H), 7.87 (d, J=8.5 Hz, 2H), 7.93 (d, J=8.5 Hz, 2H); MS (ESI): m/z 633 (M+H)⁺.

### EXAMPLE 10

### 4-{1-Benzyl-3-[3-bromo-4-(difluoro-phosphono-methyl)-benzyl]-2-oxo-2,3-dihydro-1H-imidazol-4-yl}-benzoic acid ethyl ester

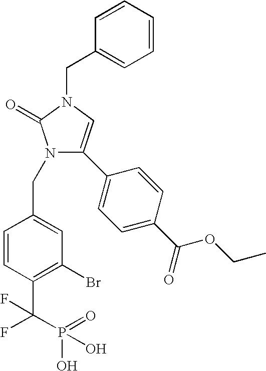

4-{1-Benzyl-3-[3-bromo-4-(difluoro-phosphono-methyl)-benzyl]-2-oxo-2,3-dihydro-1H-imidazol-4-yl}-benzoic acid methyl ester (40 mg, 0.066 mmol) was dissolved in 10 mL ethanol. One drop concentrated sulfuric acid was added, and the solution was refluxed for 24 h at which time conversion to the ethyl ester was complete. The solution was concentrated in vacuo. Water was added and the mixture was extracted thrice with 25% iPrOH/CH₂Cl₂. The combined organic extracts were washed with brine, dried (MgSO₄), then concentrated in vacuo. The resulting residue was triturated with ether to yield the title compound as a white solid, 25 mg (61%): ¹H NMR (DMSO-d₆, 500 MHz): δ (ppm) 1.30 (t, J=7.5 Hz, 3H), 4.29 (q, J=7.5 Hz, 2H), 4.88 (s, 2H), 5.03 (s, 2H), 7.11 (m, 2H), 7.30-7.40 (m, 6H), 7.46 (d, J=8.5 Hz, 2H), 7.91 (d, J=8.5 Hz, 2H); MS (ESI): m/z 622 (M+H)⁺.

### EXAMPLE 11

### ({4-[3-Benzyl-5-(4-carbamoyl-phenyl)-2-oxo-2,3-dihydro-imidazol-1-ylmethyl]-2-bromo-phenyl}-difluoro-methyl)-phosphonic acid

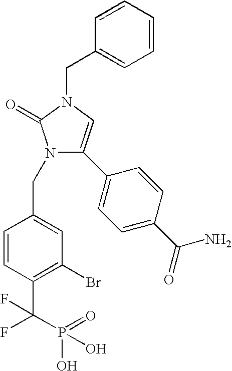

4-{1-Benzyl-3-[3-bromo-4-(difluoro-phosphono-methyl)-benzyl]-2-oxo-2,3-dihydro-1H-imidazol-4-yl}-benzoic acid methyl ester, (40 mg, 0.066 mmol) was partially dissolved in 2 mL 7.0 M ammonia in methanol. The mixture was stirred in a sealed vessel at 50° C. for one week then concentrated in vacuo. The crude product was purified by reversed phase C18-silica chromatography, eluting with 0 to 30% MeCN/H₂O to give the title compound as the bis-ammonium salt, 20 mg (48%): ¹H NMR (DMSO-d₆, 500 MHz): δ (ppm) 4.87 (s, 2H), 4.96 (s, 2H), 7.00 (m, 2H), 7.24-7.40 (m, 8H), 7.72 (d, J=8.0 Hz, 1H), 7.84 (d, J=8.5 Hz, 2H), 8.02 (bs, 1H); MS (ESI): m/z 593 (M+H)⁺.

### EXAMPLE 12

### ({4-[3-Benzyl-5-(4-methylcarbamoyl-phenyl)-2-oxo-2,3-dihydro-imidazol-1-ylmethyl]-2-bromo-phenyl}-difluoro-methyl)-phosphonic acid

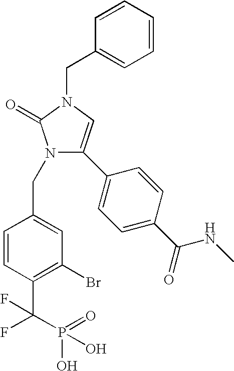

4-{1-Benzyl-3-[3-bromo-4-(difluoro-phosphono-methyl)-benzyl]-2-oxo-2,3-dihydro-1H-imidazol-4-yl}-benzoic acid methyl ester (40 mg, 0.066 mmol) was partially dissolved in 4 mL 33 wt % methylamine in ethanol. The mixture was stirred in a sealed vessel at 50° C. for three days then concentrated in vacuo. The crude product was purified by reversed phase C18-silica chromatography, eluting with 0 to 30% MeCN/H₂O to give the title compound as the bis-methylammonium salt, 20 mg (45%): ¹H NMR (DMSO-d₆, 500 MHz): δ (ppm) 2.74 (d, J=4.0 Hz, 3H), 4.87 (s, 2H), 4.96 (s, 2H), 6.99 (m, 2H), 7.23-7.40 (m, 8H), 7.68 (d, J=8.0 Hz, 1H), 7.79 (d, J=9.0 Hz, 2H), 8.57 (q, J=4.0 Hz, 1H); MS (ESI): m/z 605 (M−H)⁻.

### EXAMPLE 13

### 4-[1-Benzyl-3-(3-bromo-4-{[(2,2-dimethyl-propionyloxymethoxy)-hydroxy-phosphoryl]-difluoro-methyl}-benzyl)-2-oxo-2,3-dihydro-1H-imidazol-4-yl]-benzoic acid 2,2-dimethyl-propionyloxymethyl ester sodium salt

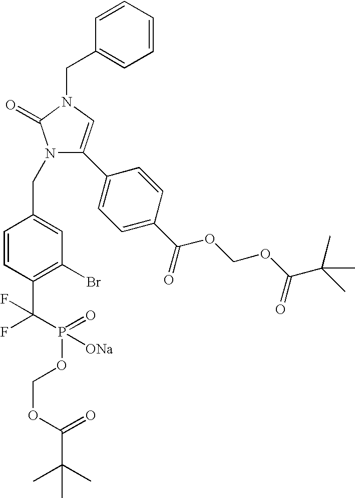

To a solution of the final product (4-{1-Benzyl-3-[3-bromo-4-(difluoro-phosphono-methyl)-benzyl]-2-oxo-2,3-dihydro-1H-imidazol-4-yl}-benzoic acid) from Example 1 ((150 mg, 0.25 mmol) and diisopropylethylamine (312 mg, 2.4 mmol) in 8 ml anhydrous dimethylformamide was added chloromethyl pivalate (365 mg, 2.4 mmol) and the mixture was heated at 70° C. for 5 hours. Solvent was removed under reduced pressure and the residue was purified on silica gel column to get 50 mg product. ¹H NMR (DMSO-d₆, 500 MHz): δ (ppm) 1.09 (s, 9H), 1.14 (s, 9H), 4.89 (s, 2H), 5.03 (s, 2H), 5.34 (d, JP-H=11.0 Hz, 2H), 5.92 (s, 2H), 7.03 (d, J=8.0 Hz, 1H), 7.14 (s, 1H), 7.27-7.40 (m, 6H), 7.49 (d, J=8.0 Hz, 2H), 7.64 (d, J=9.0 Hz, 1H), 7.91 (d, J=8.0 Hz, 1H); MS (ESI): m/z 820 (M−H)⁻.

### EXAMPLE 14

### 3-(3-Bromo-4-{[(2,2-dimethyl-propionyloxymethoxy)-hydroxy-phosphoryl]-difluoro-methyl}-benzyl)-4-(4-methoxycarbonyl-phenyl)-2-oxo-2,3-dihydro-imidazole-1-carboxylic acid ethylester

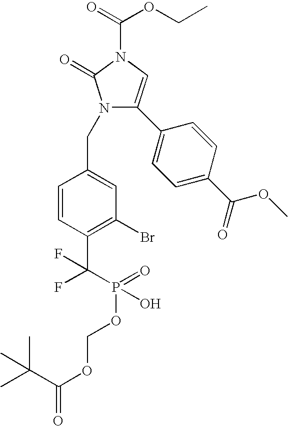

The title compound was prepared from 3-[3-Bromo-4-(difluoro-phosphono-methyl)-benzyl]-4-(4-methoxycarbonyl-phenyl)-2-oxo-2,3-dihydro-imidazole-1-carboxylic acid ethyl ester (EXAMPLE 3) as described in Example 13, white solid, (20 mg, 34%): MS (ESI): m/z 702 (M−H)⁻.

### EXAMPLE 15

### 4-{3-Benzyl-1-[3-bromo-4-(difluoro-phosphono-methyl)-benzyl]-2-oxo-2,3-dihydro-1H-imidazol-4-yl}-benzoic acid methyl ester

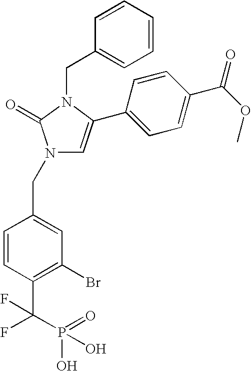

4-(3-Benzyl-1-{3-bromo-4-[(diethoxy-phosphoryl)-difluoro-methyl]-benzyl}-2-oxo-2,3-dihydro-1H-imidazol-4-yl)-benzoic acid methyl ester (132 mg, 0.20 mmol) was deprotected using iodotrimethylsilane as described for EXAMPLE 1 to give the title compound as an off-white solid, 100 mg (83%): MS (ESI): m/z 608 (M+H)⁺.

### EXAMPLE 16

### 4-{3-Benzyl-1-[3-bromo-4-(difluoro-phosphono-methyl)-benzyl]-2-oxo-2,3-dihydro-1H-imidazol-4-yl}-benzoic acid

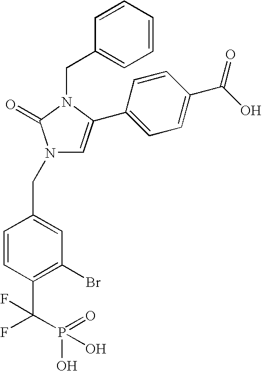

4-{3-Benzyl-1-[3-bromo-4-(difluoro-phosphono-methyl)-benzyl]-2-oxo-2,3-dihydro-1H-imidazol-4-yl}-benzoic acid methyl ester was treated with aqueous sodium hydroxide as described in EXAMPLE 2 to give the title compound as a tan solid, 35 mg (72%): MS (ESI): m/z 594 (M+H)⁺.

### EXAMPLE 17

### 4-[3-(3-Bromo-4-{[(2,2-dimethyl-propionyloxymethoxy)-hydroxy-phosphoryl]-difluoro-methyl}-benzyl)-1-methyl-2-oxo-2,3-dihydro-1H-imidazol-4-yl]-benzoic acid 2,2-dimethyl-propionyloxymethyl ester

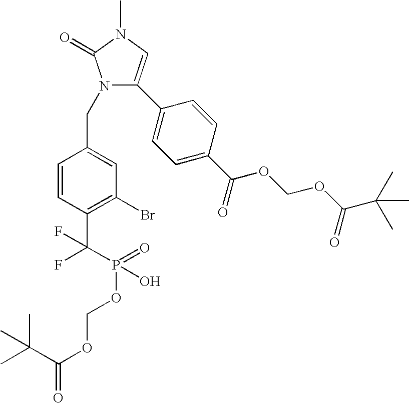

The title compound was prepared as described in Example 13, white solid (30 g, 42%). MS (ESI): m/z 745 (M−H)⁻.

### EXAMPLE 18

### 1-{[4-(difluorophosphonomethyl)-3-bromophenyl]methyl}-3-benzylimidazolidin-2-one

### N-(2,2-diethoxyethyl)[benzylamino]carboxamide

N-(2,2-diethoxyethyl)[benzylamino]carboxamide was prepared by adding slowly aminoactaldehyde diethyl ether (2.90 ml, 20.0 mmols) to a solution of benzyl isocyanate (2.47 ml, 20 mmols) in dichloromethane. The mixture was stirred for three hours, washed with sodium bicarbonate solution (2×50 ml) and brine (2×50 ml), dried over anhydrous sodium sulphate. The DCM solution was rotovaped and the residue was used in the next reaction without further purification.

### 1-benzyl-4-imidazolin-2-one

1-benzyl-4-imidazolin-2-one was prepared by dissolving N-(2,2-diethoxyethyl)[benzylamino]carboxamide from previous reaction in acetonitril (10 ml) and stirred with water (4 ml) and trifluoroacetic acid (4 ml) for 6 hours. The reaction mixture was concentrated under vacuum, dissolved in dichloromethane and washed saturated sodium bicarbonate (240 ml) and brine (2×40 ml), dried over anhydrous sodium sulphate and rotovaped and dries to get off white solid. Yield 3.0 grams.

### 1-Benzyl-imidazolidin-2-one

1-Benzyl-imidazolidin-2-one was prepared by hydrogenation of 1-benzyl-4-imidazolin-2-one (1.39 g, 8 mmols) in methanol (50 ml) in presence of 10% Pd/C (200 mg). After hydrogenation, the Pd/c was filtered and rotovaped to get 1-Benzyl-imidazolidin-2-one. Yield 1.25 grams.

### {[4-(3-Benzyl-2-oxo-imidazolidin-1-ylmethyl)-2-bromo-phenyl]-difluoro-methyl}-phosphonic acid diethyl ester

{[4-(3-Benzyl-2-oxo-imidazolidin-1-ylmethyl)-2-bromo-phenyl]-difluoro-methyl}-phosphonic acid diethyl ester was prepared by alkylating 1-Benzyl-imidazolidin-2-one (0.352 g, 2 mmols) with [(2-Bromo-4-bromo methyl-phenyl)-difluoro-methyl]-phosphonic acid diethyl ester (2.0 mmols) in presence of sodium hydride in DMF. Yield: 0.30 grams.

### 1-{[4-(difluorophosphonomethyl)-3-bromophenyl]methyl}-3-benzylimidazolidin-2-one

1-{[4-(difluorophosphonomethyl)-3-bromophenyl]methyl}-3-benzylimidazolidin-2-one was prepared by deprotecting the ethyl groups using trimethylsilyliodide method of EXAMPLE 1.

### EXAMPLE 19

### {[(2-bromo-4-{[5-(4-cyanophenyl)-2-oxo-3-benzyl(4-imidazolinyl)]methyl}phenyl)-difluoromethyl] (hydroxyphosphoryl)oxy}methyl 2,2-dimethylpropanoate, sodium salt

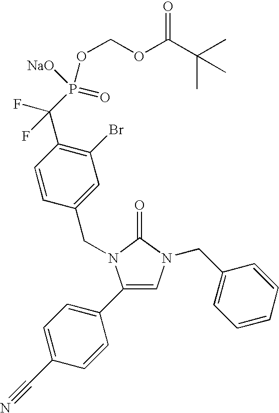

### EXAMPLE 20

### 2,2-Dimethyl-propionic acid ({4-[3-benzyl-5-(4-cyano-phenyl)-2-oxo-2,3-dihydro-imidazol-1-ylmethyl]-2-bromo-phenyl}-difluoro-methyl)-(2,2-dimethyl-propionyloxymethoxy)-phosphinoyloxymethyl ester

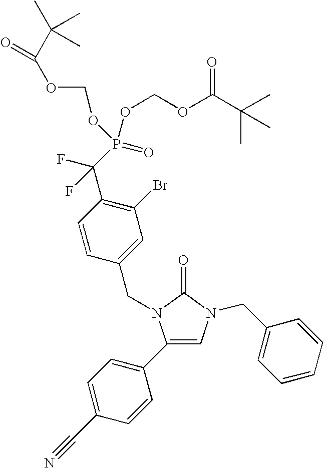

A mixture of 4-(3-{[4-(difluorophosphonomethyl)-3-bromophenyl]methyl}-2-oxo-1-benzyl-4-imidazolin-4-yl)benzenecarbonitrile (0.273 g, 0.48 mmol), chloromethyl pivalate (0.716 g, 4.75 mmol) and Cs₂CO₃ (0.310 g, 0.95 mmol) in CH₃CN (10 mL) was refluxed for overnight. The reaction mixture was partitioned between saturated NH₄Cl and ethyl acetate. The organic phase was separated, dried over Na₂SO₄ and evaporated under reduced pressure. The crude residue was suspension in 10 mL of saturated NaHCO₃ and purified by C-18 flash chromatography (CH₃CN/H₂O, 100% H₂O to 80:20) to give 0.075 g (22%) of {[(2-bromo-4-{[5-(4-cyanophenyl)-2-oxo-3-benzyl(4-imidazolinyl)]methyl}phenyl)-difluoromethyl](hydroxyphosphoryl)oxy}methyl 2,2-dimethylpropanoate, sodium salt as white solid. LC: RT, 2.1 min; MS (M+NH₄)⁺ 705, 707; (M−H)⁻ 686, 688; and 0.125 g (32%) of 2,2-Dimethyl-propionic acid ({4-[3-benzyl-5-(4-cyano-phenyl)-2-oxo-2,3-dihydro-imidazol-1-ylmethyl]-2-bromo-phenyl}-difluoro-methyl)-(2,2-dimethyl-propionyloxymethoxy)-phosphinoyloxymethyl ester as white solid. LC: RT, 3.15 min; MS (M+NH₄)⁺ 819, 821; (M-CH₂OC(═O)Me₃)⁻ 686, 688.

### EXAMPLE 21

### Methyl 4-[(3-{[4-(difluorophosphonomethyl)-3-bromophenyl]methyl}-2-oxo-4-imidazolinyl)methyl]benzoate

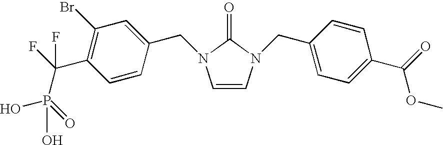

Step A. To a solution of 4-imidazolin-2-one [ref: J. Am. Chem. Soc. 1976, 8218-8221] (1.00 g, 12 mmol) in 60 mL of dry DMF was added sodium hydride (0.476 g, 12 mmol, 60%) at room temperature under an nitrogen atmosphere. After 20 minute, di-tert-butyl dicarbonate (2.60 g, 12 mmol) was added, and the solution was stirred at room temperature for overnight. The DMF was evaporated (rotavap) under vacuum. The reaction mixture was partitioned between saturated NH₄Cl and CH₂Cl₂. The organic phase was separated, dried over Na₂SO₄ and evaporated under reduced pressure. The crude residue was purified by flash chromatography (ethyl acetate/hexanes, 100% hexanes to 2:1) to isolate the tert-butyl 2-oxo-4-imidazolinecarboxylate (0.496 g) in 23% yield as white solid. MS (M−H)-183.

Step B. To a solution of tert-butyl 2-oxo-4-imidazolinecarboxylate (0.486 g, 2.65 mmol) in 20 mL of dry DMF was added potassium tert-butoxide (2.90 mL g, 2.90 mmol, 1M in tert-butanol) at room temperature under an nitrogen atmosphere. After 5 minute, {[4-(bromomethyl)-2-bromophenyl]difluoromethyl}diethoxyphosphino-1-one (1.382 g, 3.16 mmol) was injected, and the solution was stirred at room temperature for overnight. The DMF was evaporated (rotavap) under vacuum. The crude residue was purified by flash chromatography (ethyl acetate/hexanes, 100% hexanes to 2:1) to isolate the 3-{3-bromo-4-[(diethoxy-phosphoryl)-difluoro-methyl]-benzyl}-2-oxo-2,3-dihydro-imidazole-1-carboxylic acid tert-butyl ester (1.156 g) in 81% yield as colorless oil. MS (M+NH₄)⁺ 556, 558; (M-Et)⁻ 509,511.

Step C. The 3-{3-bromo-4-[(diethoxy-phosphoryl)-difluoro-methyl]-benzyl}-2-oxo-2,3-dihydro-imidazole-1-carboxylic acid tert-butyl ester (1.156 g, 2.14 mmol) in 15 mL of 4M solution of HCl in 1,4-dioxane, and the solution was stirred at room temperature for 2 hours. The reaction was then rotary evaporated to an oil which was dissolved in ethyl acetate and washed with saturated NaHCO₃ followed by a wash with saturated NaCl. The organic layer was dried (Na₂SO₄) and concentrated to yield 0.870 g (93%) of {[2-bromo-4-(2-oxo-2,3-dihydro-imidazol-1-ylmethyl)-phenyl]-difluoro-methyl}-phosphonic acid diethyl ester as yellow oil. MS (M+NH₄)⁺ 456, 458; (M−H)⁻ 437, 439.

Step D. To a solution of {[2-bromo-4-(2-oxo-2,3-dihydro-imidazol-1-ylmethyl)-phenyl]-difluoro-methyl}-phosphonic acid diethyl ester (0.451 g, 1.03 mmol) in 20 mL of dry DMF was added potassium tert-butoxide (1.13 mL g, 1.13 mmol, 1M in tert-butanol) at room temperature under an nitrogen atmosphere. After 5 minute, methyl 4-(bromomethyl)benzoate (0.282 g, 1.23 mmol) was added, and the solution was stirred at room temperature for overnight. The DMF was evaporated (rotavap) under vacuum. The crude residue was purified by flash chromatography (ethyl acetate/hexanes, 100% hexanes to 4:1) to isolate the 4-(3-{3-Bromo-4[(diethoxy-phosphoryl)-difluoro-methyl]-benzyl}-2-oxo-2,3-dihydro-imidazol-1-ylmethyl)-benzoic acid methyl ester (0.316 g) in 52% yield as colorless oil. MS (M+NH₄)⁺ 604, 606; (M−Et)⁻ 557,559.

Step E. A solution of 4-(3-{3-Bromo-4-[(diethoxy-phosphoryl)-difluoro-methyl]-benzyl}-2-oxo-2,3-dihydro-imidazol-1-ylmethyl)-benzoic acid methyl ester (0.296 g, 0.50 mmol) and BSTFA (1.61 mL, 6.05 mmol) in dry dichloromethane (5 mL) was stirred at room temperature for 1 hour. The reaction mixture was cool to −20° C., then added iodotrimethylsilane (0.57 mL, 4.03 mmol). The reaction mixture was slowly warmed up to room temperature to stir for an additional 2 hour. The solvent and excess iodotrimethylsilane was removed by vacuum. The residue was treated with acetonitrile (16 mL) and H₂O (4 mL), and stirred for overnight at room temperature. The reaction was then rotary evaporated to an oil which was dissolved in ethyl acetate and washed with 5% Na₂S₂O₄ (acidified) followed by a wash with saturated NaCl. The organic layer was dried (Na₂SO₄) and concentrated. The crude residue was purified by C-18 flash chromatography (MeOH/H₂O, 100% H₂O to 3:7) to give the methyl 4-[(3-{[4-(difluorophosphonomethyl)-3-bromophenyl]methyl}-2-oxo-4-imidazolinyl)methyl]benzoate (0.233 g) in 87% yield as white solid. MS (M+NH₄)⁺ 548, 550; (M−H)⁻ 529, 531.

### EXAMPLE 22

### 4-[(3-{[4-(difluorophosphonomethyl)-3-bromophenyl]methyl}-2-oxo-4-imidazolinyl)methyl]benzoic acid

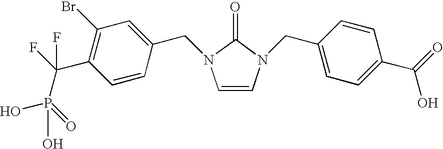

The methyl 4-[(3-{[4-(difluorophosphonomethyl)-3-bromophenyl]methyl}-2-oxo-4-imidazolinyl)methyl]benzoate (0.075 g, 0.14 mmol) was dissolved in 20 mL of 0.25 M LiOH solution (in MeOH/H₂O, 75/25). The mixture was stirred overnight at room temperature and concentrated in vacuo. The residue added water, then added 1 N HCl aq. solution until pH<7. The crude residue was purified by C-18 flash chromatography (MeCN/H₂O, 100% H₂O to 2:8) to give the 4-[(3-{[4-(difluorophosphonomethyl)-3-bromophenyl]methyl}-2-oxo-4-imidazolinyl)methyl]benzoic acid (0.070 g) in 96% yield as white solid. MS (M+NH₄)⁺ 534, 536; (M−H)⁻ 515, 517.

### EXAMPLE 23

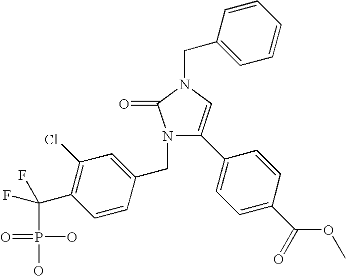

### 4-{1-Benzyl-3-[3-chloro-4-(difluoro-phosphono-methyl)-benzyl]-2-oxo-2,3-dihydro-1H-imidazol-4-yl}-benzoicacid methyl ester

This compound was prepared employing the procedure used in Example 1 using [(2-chloro-4-methyl-phenyl)-difluoromethyl]-phosphonic acid diethyl ester instead of [(2-bromo-4-methyl-phenyl)-difluoromethyl]-phosphonic acid diethyl ester. MS (ESI): m/z 561 (M−H)⁺.

### EXAMPLE 24

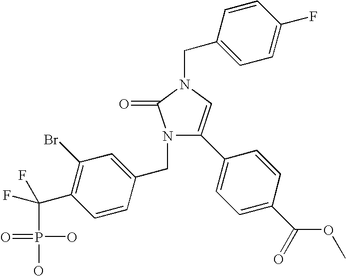

### 4-[3-[3-Bromo-4-(difluoro-phosphono-methyl)-benzyl]-1-(4-fluoro-benzyl)-2-oxo-2,3-dihydro-1H-imidazol-4-yl]-benzoic acid methyl ester

This compound was prepared employing the procedure as described in Example 1 using 4-fluorobenzylamine instead of benzylamine. MS (ESI): m/z 624 (M−H)⁺.

### EXAMPLE 25

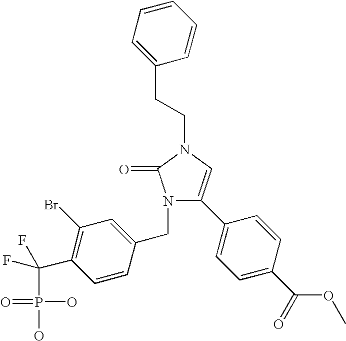

### 4-[3-[3-Bromo-4-(difluoro-phosphono-methyl)-benzyl]-1-(4-fluoro-benzyl)-2-oxo-2,3-dihydro-1H-imidazol-4-yl]-benzoic acid methyl ester

This compound was prepared employing the procedure as described in Example 1 using phenethylamine instead of benzylamine. MS (ESI): m/z 620 (M−H)⁺.

### EXAMPLE 26

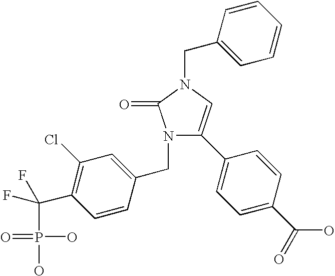

### 4-{1-Benzyl-3-[3-chloro-4-(difluoro-phosphono-methyl)-benzyl]-2-oxo-2,3-dihydro-1H-imidazol-4-yl}-benzoic acid

This compound was prepared employing the procedure as described in Example 1 using [(2-chloro-4-methyl-phenyl)-difluoromethyl]-phosphonic acid diethyl ester instead of [(2-bromo-4-methyl-phenyl)-difluoromethyl]-phosphonic acid diethyl ester. MS (ESI): m/z 547 (M−H)⁺.

### EXAMPLE 27

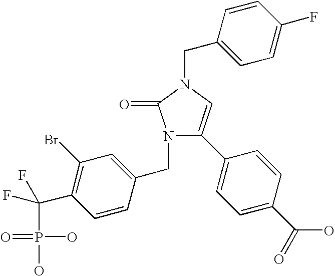

### 4-{1-Benzyl-3-[3-Bromo-4-(difluoro-phosphono-methyl)-benzyl]-2-oxo-2,3-dihydro-1H-imidazol-4-yl}-benzoic acid

This compound was prepared employing the procedure of EXAMPLE 1 using 4-fluorobenzylamine instead of benzylamine. MS (ESI): m/z 609 (M−2H)⁺.

### EXAMPLE 28

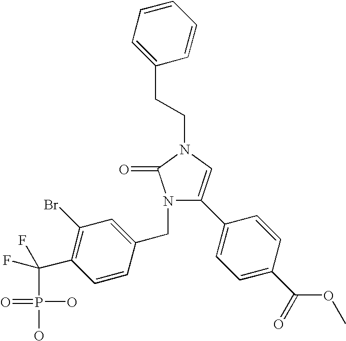

### 4-{3-[3-Bromo-4-(difluoro-phosphono-methyl)-benzyl]-2-oxo-1-phenethyl-2,3-dihydro-1H-imidazol-4-yl}-benzoic acid

This compound was prepared employing the procedure used in EXAMPLE 1 using phenethylamine instead of benzylamine. MS (ESI): m/z 606 (M−H)⁺.

### EXAMPLE 29

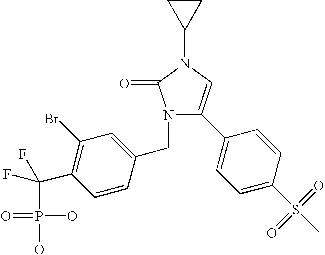

### 2-Bromo-1-(4-methanesulfonyl-phenyl)-ethanone)

To a solution of 1-(4-Methanesulfonyl-phenyl)-ethanone (25 g, 0.12 mol) in 500 mL chloroform was added, drop-wise, bromine (19.2 g, 0.12 mol) in 60 mL chloroform over a period of 3 h. The mixture was stirred over night. Water (200 mL) was carefully added to the reaction mixture, mixed well and the organic layer isolated was washed with saturated sodium hydrogen carbonate in water (200 mL), followed by brine (200 mL), dried over anhydrous sodium sulfate, filtered and filtrate evaporated to get pure product as a light yellow powder (32 g, 87%).

### 1-Cyclopropyl-4-(4-methanesulfonyl-phenyl)-1,3-dihydro-imidazol-2-one

To an ice cooled mixture of cyclopropyl amine (1.01 mg, 18 mmol) and triethylamine (334 mg, 3.3 mmol) in 20 mL chloroform was added, drop-wise, a solution 2-Bromo-1-(4-methanesulfonyl-phenyl)-ethanone (831 mg, 3 mmol) in 10 mL chloroform. After allowing the mixture to warm to room temperature, the mixture was stirred at room temperature for 2 hours. The mixture was diluted with 20 mL dichloromethane and the washed with water (2×50 mL), dried over anhydrous sodium sulfate, filtered and evaporated. The residue was dissolved in 12 mL methanol, cooled to 0° C., 4 mL glacial acetic acid was added followed by potassium cyanate (364 mg). The mixture was refluxed for 1.5 and cooled. The precipitate formed was isolated to get 300 mg pure title compound.

### ({2-Bromo-4-[3-cyclopropyl-5-(4-methanesulfonyl-phenyl)-2-oxo-2,3-dihydro-imidazol-1-ylmethyl]-phenyl}-difluoro-methyl)-phosphonic acid diethyl ester

To a suspension of sodium hydride (95%, 22 mg, 0.86 mmol) in anhydrous dimethylformamide (3 mL) was added (200 mg, 0.72 mmol) of 1-Cyclopropyl-4-(4-methanesulfonyl-phenyl)-1,3-dihydro-imidazol-2-one and stirred for 15 minutes. To this solution was added a solution of [(2-Bromo-4-bromomethyl-phenyl)-difluoro-methyl]-phosphonic acid diethyl ester (376 mg, 0.86 mmol) in 1 mL anhydrous dimethylformamide slowly. The mixture was stirred at room temperature for 6 h. Solvent was removed under reduced pressure and the residue dissolved in 100 mL ethyl acetate was washed with water (2×20 mL), dried over anhydrous sodium sulfate, filtered and evaporated. The residue obtained was loaded on a silica gel column and purified using hexane and 1:1 hexane:ethyl acetate to elute the impurities followed by 2:1 ethyl acetate:hexane to elute the compound. Fractions collected were pooled and evaporated to obtain 115 mg (25%) of pure product.

### ({2-Bromo-4-[3-cyclopropyl-5-(4-methanesulfonyl-phenyl)-2-oxo-2,3-dihydro-imidazol-1-ylmethyl]-phenyl}-difluoro-methyl)-phosphonic acid

To ({2-Bromo-4-[3-cyclopropyl-5-(4-methanesulfonyl-phenyl)-2-oxo-2,3-dihydro-imidazol-1-ylmethyl]-phenyl}-difluoro-methyl)-phosphonic acid diethyl ester (105 mg, 0.16 mmol) in CH₂Cl₂ (50 mL) was added bistrimethylsilyltrifluoroacetamide (213 mg, 0.83 mmol) and reaction mixture was stirred at room temperature for 1 h after which the reaction mixture is cooled to −20° C. and iodotrimethylsilane (166 mg, 0.83 mmol) was added drop-wise. The resulting mixture was stirred at room temperature for 1.5 hours, after which it was concentrated in vacuo. The residue was dissolved in 25 mL dichloromethane and cooled in ice-bath. To this solution was added methanol (25 mL) slowly. The mixture was stirred at room temperature for 2 h and then solvent was removed under reduced pressure. The residue was dissolved in 5 mL methanol and to this was added 150 mL ethyl acetate and washed with freshly acidified (pH<1) 2.5% solution (40 mL) of sodium dithionite in water followed by brine (40 mL). The organic solution was dried over anhydrous sodium sulfate, filtered and evaporated under reduced pressure to obtain 72 mg (78%) of product. MS (ESI): m/z 576 (M−H)⁺.

### EXAMPLE 30

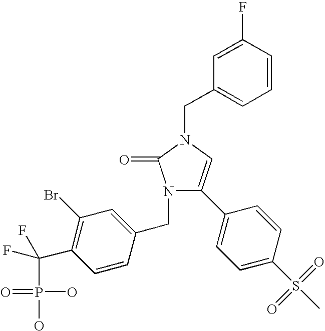

### ({2-Bromo-4-[3-(3-fluoro-benzyl)-5-(4-methanesulfonyl-phenyl)-2-oxo-2,3-dihydro-imidazol-1-ylmethyl]-phenyl}-difluoro-methyl)-phosphonic acid

This compound was prepared employing the procedure of Example 29 using 3-fluorobenzylamine instead of benzylamine. MS (ESI): m/z 644 (M−H)⁺.

### EXAMPLE 31

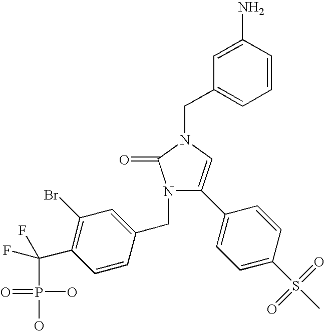

### Step 1. ({2-Bromo-4-[5-(4-methanesulfonyl-phenyl)-3-(3-nitro-benzyl)-2-oxo-2,3-dihydro-imidazol-1-ylmethyl]-phenyl}-difluoro-methyl)-phosphonic acid diethyl ester

This compound was prepared according to the general procedure for making similar substituted imidazolone as described in Example 29.

### Step 2. ({4-[3-(3-Amino-benzyl)-5-(4-methanesulfonyl-phenyl)-2-oxo-2,3-dihydro-imidazol-1-ylmethyl]-2-bromo-phenyl}-difluoro-methyl)-phosphonic acid diethyl ester

Compound from Step 1 (380 mg, 0.52 mmol) and tin (II) chloride dihydrate (0.589 mg, 2.60 mmol) were refluxed in ethanol (6 ml) for 3 h. After evaporation of solvent under reduced pressure the residue was suspended in 50 ml ethyl acetate, washed with saturated aqueous sodium bicarbonate (30 ml), reextracted the bicarbonate wash with ethyl acetate 92×40 ml), combined extracts was dried (anhydrous sodium sulfate), filtered and evaporated to get pure product (330 mg, 91%).

### Step 3. ({4-[3-(3-Amino-benzyl)-5-(4-methanesulfonyl-phenyl)-2-oxo-2,3-dihydro-imidazol-1-ylmethyl]-2-bromo-phenyl}-difluoro-methyl)-phosphonic acid

Compound from Step 2 was subjected to deprotection using bistrifluoromethlsilyl-acetamide and iodomethylsilane as employed in EXAMPLE 1 to get the title product. MS (ESI): m/z 641 (M−H)⁺.

### EXAMPLE 32

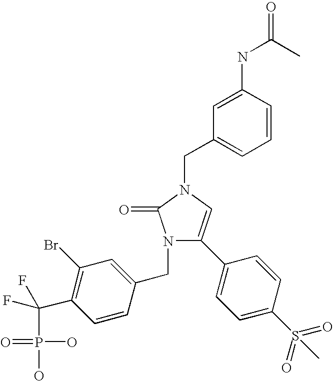

### Step 1. ({4-[3-(3-Acetylamino-benzyl)-5-(4-methanesulfonyl-phenyl)-2-oxo-2,3-dihydro-imidazol-1-ylmethyl]-2-bromo-phenyl}-difluoro-methyl)-phosphonic acid diethyl ester

Product form Step 2 of EXAMPLE 31 (236 mg, 0.34 mmol), acetic anhydride (69 mg, 0.68 mmol), triethylamine (69 mg, 0.68 mmol) and catalytic amount of N,N-dimethylaminopyridine were stirred in 4 ml anhydrous dichloromethane at room temperature over night. The mixture was diluted with 25 ml dichloromethane, washed with water (10 ml), saturated aqueous sodium bicarbonate (10 ml) and brine (10 ml), dried over sodium sulfate, filtered and evaporated. The residue was purified on silica gel column to get 86 mg pure product.

### Step 2. ({4-[3-(3-Acetylamino-benzyl)-5-(4-methanesulfonyl-phenyl)-2-oxo-2,3-dihydro-imidazol-1-ylmethyl]-2-bromo-phenyl}-difluoro-methyl)-phosphonic acid

The product from previous step was subjected to deprotection using bistrifluoromethlsilylacetamide and iodomethylsilane as employed in Example 1 to get the title product. MS (ESI): m/z 683 (M−H)⁺.

### EXAMPLE 33

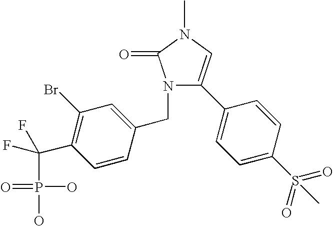

### Step 1. ({2-Bromo-4-[5-(4-methanesulfonyl-phenyl)-3-methyl-2-oxo-2,3-dihydro-imidazol-1-ylmethyl]-phenyl}-difluoro-methyl)-phosphonic acid diethyl ester

To 3-{3-Bromo-4-[(diethoxy-phosphoryl)-difluoro-methyl]-benzyl}-4-(4-methanesulfonyl-phenyl)-2-oxo-2,3-dihydro-imidazole-1-carboxylic acid ethyl ester (405 mg, 0.609 mmol) in anhydrous THF (4 ml) was added 2.5 ml of 1M dimethylamine in THF at 0° C. and stirred for 1 hour. Solvent was evaporated under reduced pressure and the residue was dried under vacuum to get 353 mg product.

Step 2. To the product from previous step (117 mg, 0.197 mmol) in 3 ml anhydrous N,N-dimethylformamide at 0° C. was added 1M potassium tert-butoxide in tert-butanol (0.3 ml) followed by iodomethane drop-wise. The mixture was stirred for 20 minutes. Solvent was removed and the residue was purified on silica gel column to get 100 mg product.

Step 3. Product from previous step was subjected to deprotection using standard protocol (BSTFA/TMSI) as described in Example 1 to get the title product. MS (ESI): m/z 550 (M−H)⁺.

### EXAMPLE 34

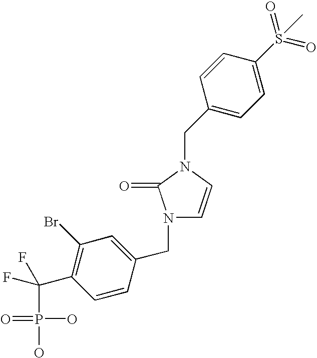

### Step 1. ({2-Bromo-4-[3-(4-methanesulfonyl-benzyl)-2-oxo-2,3-dihydro-imidazol-1-ylmethyl]-phenyl}-difluoro-methyl)-phosphonic acid diethyl ester

To a solution of triphosgene (384 mg, 1.29 mmol) in 10 ml anhydrous dichloromethane was added, drop-wise, a solution of 4-methanesulfonylbenzyleamine (776 mg, 3.5 mmol) and diisopropylethylamine (498 mg, 3.85 mmol) in 10 ml anhydrous dichloromethane over a period of 1 hours at room temperature. After 5 minutes of additional stirring, a solution of aminoacetaldehyde diethylacetal (466 mg, 3.5 mmol) and diisoprpylethylamine (498 mg, 3.85 mmol) in 10 ml anhydrous dichloromethane was added in one portion and stirred at room temperature for 5 h. The mixture was washed with 20 ml each of saturated aqueous sodium bisulfate and sodium bicarbonate and evaporated under reduced pressure. The residue was dissolved in a mixture of 5 ml acetonitrile and 1 ml water and 1 ml trifluoroacetic acid was added to this. This mixture was stirred at room temperature over night. Solvent was evaporated and the residue dissolved in dichloromethane was washed with saturated sodium bicarbonate in water, dried over sodium sulfate, filtered and evaporated. The residue was purified on silica gel column to get 135 mg product.

### Step 2. ({2-Bromo-4-[3-(4-methanesulfonyl-benzyl)-2-oxo-2,3-dihydro-imidazol-1-ylmethyl]-phenyl}-difluoro-methyl)-phosphonic acid

Product from previous step was deprotected using standard protocol (BSTFA/TMSI) as described in Example 1 to get the title product. MS (ESI): m/z 550 (M−H)⁺.

### EXAMPLE 35

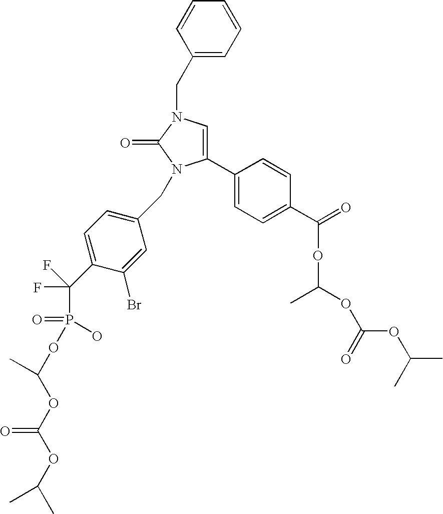

### 4-[1-Benzyl-3-(3-bromo-4-{difluoro-[hydroxy-(1-isopropoxycarbonyloxy-ethoxy)-phosphoryl]-methyl}-benzyl)-2-oxo-2,3-dihydro-1H-imidazol-4-yl]-benzoic acid 1-isopropoxycarbonyloxy-ethyl ester

To a solution of 4-{1-Benzyl-3-[3-bromo-4-(difluoro-phosphono-methyl)-benzyl]-2-oxo-2,3-dihydro-1H-imidazol-4-yl}-benzoic acid (275 mg, 0.46 mmol) in 5 mL N,N-dimethylformamide under nitrogen atmosphere was added triethylamine (0.52 mL, 3.71 mmol) followed by 1-chloroethyl isopropyl carbonate (617.7 mg, 3.71 mmol) followed by tetra-n-butyl ammonium iodide (68 mg). The mixture was stirred at 80° C. for 20 hours. Solvent was evaporated under reduced pressure. The residue was purified on silica gel column to obtain 127 mg (32%) of title compound. MS (ESI): m/z 851 (M−H)⁺.

### EXAMPLE 36

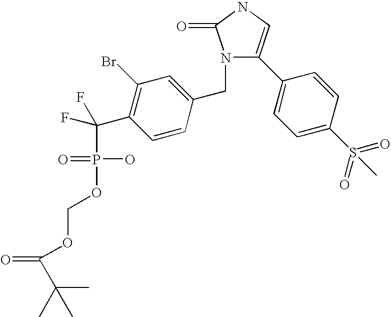

### Step 1. 3-(3-Bromo-4-{[(2,2-dimethyl-propionyloxymethoxy)-hydroxy-phosphoryl]-difluoro-methyl}-benzyl)-4-(4-methanesulfonyl-phenyl)-2-oxo-2,3-dihydro-imidazole-1-carboxylic acid ethylester

To a solution of 3-[3-Bromo-4-(difluoro-phosphono-methyl)-benzyl]-4-(4-methanesulfonyl-phenyl)-2-oxo-2,3-dihydro-imidazole-1-carboxylic acid ethyl ester (150 mg, 0.24 mmol) and diisopropyethylamine (156.4 mg, 1.21 mmol) in 4 ml anhydrous dimethylformamide was added chloromethyl pivalate (182.5 mg, 1.21 mmol) and the mixture was heated at 70° C. for 5 hours. Solvent was removed under reduced pressure and the residue was purified on silica gel column to get 60 mg product.

### Step 2. 2,2-Dimethyl-propionic acid ({2-bromo-4-[5-(4-methanesulfonyl-phenyl)-2-oxo-2,3-dihydro-imidazol-1-ylmethyl]-phenyl}-difluoro-methyl)-hydroxy phosphinoyloxymethyl ester

Product from previous step was deprotected employing the protocol used in Example 6 to get the title product. MS (ESI): m/z 649 (M−H)⁺.

### EXAMPLE 37

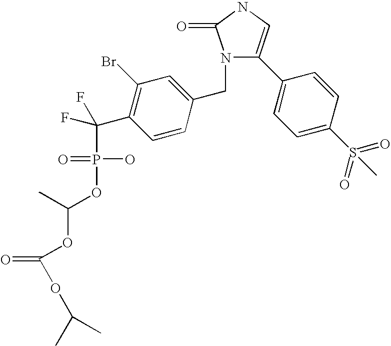

### Step 1. 3-(3-Bromo-4-{difluoro-[hydroxy-(1-isopropoxycarbonyloxy-ethoxy)-phosphoryl]-methyl}-benzyl)-4-(4-methanesulfonyl-phenyl)-2-oxo-2,3-dihydro-imidazole-1-carboxylic acid ethyl ester

This compound was prepared from 3-[3-Bromo-4-(difluoro-phosphono-methyl)-benzyl]-4-(4-methanesulfonyl-phenyl)-2-oxo-2,3-dihydro-imidazole-1-carboxylic acid ethyl ester and 1-chloroethyl isopropyl carbonate employing the same procedure used in Example 35.

### Step 2. ({2-Bromo-4-[5-(4-methanesulfonyl-phenyl)-2-oxo-2,3-dihydro-imidazol-1-ylmethyl]-phenyl}-difluoro-methyl)-phosphonic acid mono-(1-isopropoxycarbonyloxy-ethyl) ester

Product from previous step was deprotected employing the protocol as described in Example 6 to get the title product. MS (ESI): m/z 665 (M−H)⁺.

### EXAMPLE 38

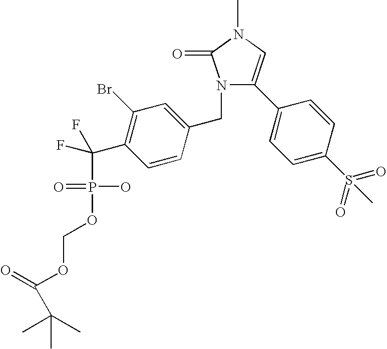

### 2,2-Dimethyl-propionic acid ({2-bromo-4-[5-(4-methanesulfonyl-phenyl)-3-methyl-2-oxo-2,3-dihydro-imidazol-1-ylmethyl]-phenyl}-difluoro-methyl)-hydroxyphosphinoyloxymethyl ester

This compound was prepared from ({2-Bromo-4-[5-(4-methanesulfonyl-phenyl)-3-methyl-2-oxo-2,3-dihydro-imidazol-1-ylmethyl]phenyl}-difluoro-methyl)-phosphonic acid and chloromethyl pivalate employing the protocol as described in Example 36. MS (ESI): m/z 663 (M−H)⁺.

### EXAMPLE 39

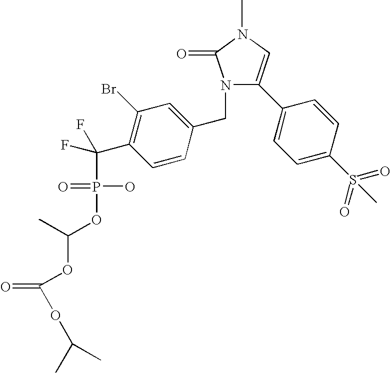

### ({2-Bromo-4-[5-(4-methanesulfonyl-phenyl)-3-methyl-2-oxo-2,3-dihydro-imidazol-1-ylmethyl]-phenyl}-difluoro-methyl)-phosphonic acid mono-(1-isopropoxycarbonyloxy-ethyl) ester

This compound was prepared from ({2-Bromo-4-[5-(4-methanesulfonyl-phenyl)-3-methyl-2-oxo-2,3-dihydro-imidazol-1-ylmethyl]phenyl}-difluoro-methyl)-phosphonic acid and 1-chloroethyl isopropyl carbonate employing the protocol as described in Example 35. MS (ESI): m/z 679 (M−H)⁺.

### EXAMPLE 40

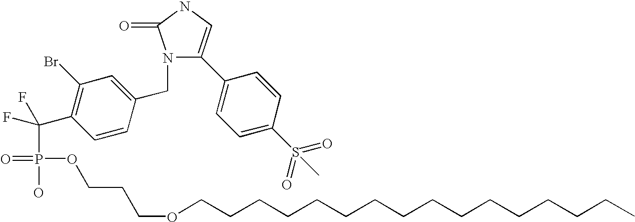

### ({2-Bromo-4-[5-(4-methanesulfonyl-phenyl)-2-oxo-2,3-dihydro-imidazol-1-ylmethyl]-phenyl}-difluoro-methyl)-phosphonic acid mono-(3-hexadecyloxy-propyl) ester

This compound was prepared employing the procedure of Example 36 using 1-(3-chloro-propoxy)-hexadecane as the alkylating agent. MS (ESI): m/z 702 (M−H)⁺.

### EXAMPLE 41

### Assay for PTP-1B Activity

Materials:

EDTA—ethylenediaminetetraacetic acid (Sigma)

HEPES—N-(2-Hydroxyethyl)piperazine-N′-(2-ethanesulfonic acid) (Sigma)

DTT—dithiothreitol (Sigma)

NaCl—sodium chloride

Enzyme: Human recombinant PTP-1B, containing amino acids 1-321, fused to GST enzyme (glutathione S-transferase) purified by affinity chromatography (Huyer et al, 1997, J. Biol. Chem., 272, 843-852).

pNPP: p-Nitrophenyl Phosphate (Calbiochem)

Assay Buffer: 50 mM HEPES (pH 7.4), 100 mM NaCl, 2 mM EDTA, 3 mM DTT

(5×) pNPP substrate: 10 mM pNPP in assay buffer

Enzyme: 1 mg/ml PTP1B (1-321), dilute 1:200 in assay buffer

Enzyme Assay PTP-1B

Assay buffer: 50 mM HEPES (pH 7.4), 100 mM NaCl, 2 mM EDTA, 3 mM DTT

Enzyme dilution buffer: 50 mM HEPES (pH 7.4), 100 mM NaCl, 2 mM EDTA, 3 mM DTT

Substrate 198 mM p-Nitrophenyl Phosphate (pNPP) store at 4° C.

The assay was carried out at 30° C. in 96 well plates. The reaction mixture in 60 ul contained 50 mM HEPES (pH 7.4), 100 mM NaCl, 2 mM EDTA, 3 mM DTT, and 2 mM p-Nitrophenyl Phosphate (pNPP). 5 ul of the test compound (inhibitor) dissolved in DMSO or DMSO alone for control was added to each well and the plate was mixed for 2 min. The reaction was initiated by adding 20 ul of diluted PTP1B (5 ng/ul in 50 mM HEPES (pH 7.4), 100 mM NaCl, 2 mM EDTA, 3 mM DTT). After 20 min at 30° C., the enzyme reaction was terminated by adding 100 ul of 2 M K2CO3 to each well. The phosphatase activity was detected by using Victor II plate reader (Wallac) with absorbance detection at 405 nm. All the assays were done at least in duplicate. The initial rate of pNP is plotted against the concentration of inhibitor.

### EXAMPLE 42

### Cell Based Assays

Antibodies and Chemicals. The antibody against phosphorylated insulin receptor (pIR) and the ELISA kit for detection of pIR were from Biosource (Camarillo, Calif.). Rabbit anti-IR/IGF-1R [pYpY1162/1163] phosphospecific antibody recognizes both the insulin receptor (IR) and the insulin-like growth factor-1 receptor (IGF-1R) phosphorylated at the active site tyrosine residues, 1162 and 1163 (1135 and 1136 for IGF-1R) (pIR/pIGF-1R). The Insulin Receptor [pYpY1162/1163] ELISA kit specifically recognizes IR phosphorylated at tyrosine residues 1162 and 1163 (and does not recognize phosphorylated IGF-1R). HRP-conjugated secondary antibodies were from Cell Signaling Technology (Beverly, Mass.). The ECL detection system was from Amersham (Buckinghamshire, UK), and human insulin was from Invitrogen (Carlsbad, Calif.).

Tissue Culture. FAO rat hepatoma cells were obtained from ECACC (#89042701) and maintained at 37° C. in a 5% CO₂ environment in Dulbecco's modified Eagle's medium with high glucose (DMEM-high glucose) (4500 mg/liter) supplemented with 10% FBS and 50 units/ml penicillin, 100 μg/ml streptomycin and 0.292 mg/ml L-glutamine. For assays, cells were seeded in 24-well plates at a density of 2×10⁵ cells/well and maintained until they reached confluency (about 3 days).

IR phosphorylation assays. Cells in 24-well plates were serum starved overnight in DMEM-low glucose (1000 mg/liter) without serum. Just before use, the starvation medium was discarded and replaced with 0.5 ml of DMEM without serum. Cells were treated for 1 hour with indicated concentrations of compounds, followed by stimulation with or without insulin for 15-30 minutes. For Western analysis, the reaction was stopped by discarding the medium and adding 80 μl of boiling SDS sample lysis buffer [62.5 mM Tris-HCl (pH 6.8), 50 mM DTT, 2% w/v SDS, 10% glycerol, 50 mM NaF, 1 mM Na₃VO₄, 2 mM pNPP, 20 mM P-glycerol phosphate and 0.1% w/v bromophenol blue]. 20 μl of the lysates were loaded onto 4-20% Tris-Glycine grandient gels (Invitrogen, Carlsbad, Calif.) and the proteins resolved by SDS-PAGE and transferred to nitrocellulose membranes. The membranes were probed for detection of pIR/pIGF-1 R and total PTP-1B using the ECL chemiluminescence detection system. The pIR/pIGF-1R signals were scanned (HP scanjet 3570c) and quantified (Scion Image). For ELISA analysis, the medium was discarded and the plates placed onto a dry ice/ethanol bath for 3 minutes to stop the reaction, then placed on ice. The cells were then lysed and processed according to the ELISA instruction kit manuals for detection of pIR and pAkt (Biosource, Camarillo, Calif.)

Since modifications will be apparent to those of skill in the art, it is intended that the invention be limited only by the scope of the appended claims.

1.-89. (canceled)

90. A compound of formulae I: 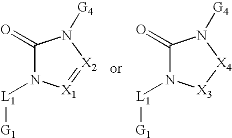
or a pharmaceutically acceptable derivative thereof, wherein:
X₁ and X₂ are each, independently, N, CG₂ or CG₃, with the proviso that only one of X₁ and X₂ can be N;
X₃ and X₄ are each, independently, NR or CG₂G₃, where R is H, alkyl, alkenyl, alkynyl, cycloalkyl, heterocyclyl, aryl or heteroaryl, with the proviso that only one of X₃ and X₄ can be NR;
L₁ is a linker;
G₁ is: 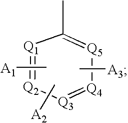
G₂ and G₃ are selected from (i) or (ii) as follows:
(i) G₂ is H, alkyl or: 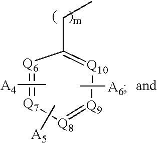
G₃ is H, alkyl, alkoxy, F, Br, Cl or: 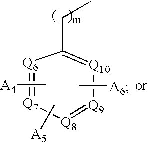
(ii) G₂ and G₃, together with the atoms to which they are attached, form a 5-7 membered cycloalkyl, heterocyclyl, aryl or heteroaryl ring;
G₄ is H, alkyl, cycloalkyl, COOalkyl, cycloalkylalkylor: 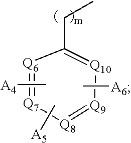
m is an integer from 0 to 2;
Q₁ through Q₁₀ are independently selected from no bond (direct link), C, N, S, and O, with the proviso that the resulting combination of atoms is a chemically stable cyclic and/or (hetero)aromatic ring system, and where if the Q is C, then it is substituted with one of A₁-A₆ or hydrogen, and where if the Q is N, S or O, it is not substituted with one of A₁-A₆;
where A1-A6 are each independently selected from (i)-(ix) as follows:
(i) no substituent, H, F, Cl, Br, I, CF₃, CF₂CF₃, CH₂CF₃, CF₂CH₃, OH, OCF₃, OCHCl₂, CN, NO₂, C₁-C₆-alkyl which is unsubstituted or mono-, di- or tri-substituted with 1 to 3 substituents selected from the group consisting of Y₁, Y₂, and Y₃, C₂-C₆-alkenyl which is unsubstituted or mono-, di- or tri-substituted with 1 to 3 substituents selected from the group consisting of Y₁, Y₂, and Y₃, C₂-C₆-alkynyl which is unsubstituted or mono-, di- or tri-substituted with 1 to 3 substituents selected from the group consisting of Y₁, Y₂, and Y₃, C₁-C₆ alkoxy which is unsubstituted or mono-, di- or tri-substituted with 1 to 3 substituents selected from the group consisting of Y₁, Y₂, and Y₃, C₃-C₆ alkenyloxy which is unsubstituted or mono-, di- or tri-substituted with 1 to 3 substituents selected from the group consisting of Y₁, Y₂, and Y₃, C₃-C₆ alkynyloxy which is unsubstituted or mono-, di- or tri-substituted with 1 to 3 substituents selected from the group consisting of Y₁, Y₂, and Y₃, C₃-C₈-cycloalkyl which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃; or two adjacent A groups may be joined together to form a fused cycloalkyl, heterocyclyl, heteroaromatic or aromatic ring;
(ii) P(═O)(OR)(OR1), P(═O)(OH)₂, P(═O)(OH)(OCH₃), P(═O)(OH)(OC₂H₅), P(═O)(OR)[(OCRR1)OC(═O)R], P(═O)(OR)[(OCRR 1)OC(═O)OR], P(═O)[(OCRR1)OC(═O)R)]₂, P(═O)[(OCRR1)OC(═O)OR)]₂, P(═O)(OR)(OR1), P(═O)(Me)(OR), P(═O)(CF₃)(OR), P(═O)(Me)(NHR), P(═O)(NHR)(OR), P(═O)(NHR)(NHR1), CR═CR—P(═O)(OR)(OR1), CR═CR—P(═O)(Me)(OR), CR═CR—P(═O)(CF₃)(OR), CR═CR—P(═O)(Me)(NHR), CR═CR—P(═O)(NHR)(OR), CR═CR—P(═O)(NHR)(NHR1), [CH(OH)]_{q}P(═O)(OR)(OR1), [CH(OH)]_{q}P(═O)(Me)(OR1), [CH(OH)]_{q}P(═O)(CF₃)(OR1), CC—P(═O)(OR)(OR1), CC—P(═O)(Me)(OR), CC—P(═O)(CF₃)(OR), CC—(CF₂)_{q}—P(═O)(OR)(OR1), CC—(CF₂)_{q}—P(═O)(Me)(OR1), CC—(CF₂)_{q}—P(═O)(CF₃)(OR1), [CH(OH)]_{q}CF₂P(═O)(OR)(OR1) [CH(OH)]_{q}(CF₂)_{q}P(═O)(Me)(OR1), [CH(OH)]_{q}(CF₂)_{q}P(═O)(CF₃)(OR1), (CF₂)_{q}P(═O)(OR)(OR1), (CF₂)_{q}P(═O)(Me)(OR), (CF₂)_{q}P(═O)(CF₃)(OR), (CF₂)_{q}P(═O)(Me)NHR, (CF₂)_{q}P(═O)(NHR)(OR), (CFR)_{q}P(═O)(OR)(OR1), (CFR)_{q}P(═O)(Me)(OR), —(CFR)_{q}P(═O)(CF₃)(OR), (CFR)_{q}P(═O)(Me)NHR, (CF₂)_{q}P(═O)(NHR)(OR), CF═CF—P(═O)(OR)(OR1), CF═CF—P(═O)(Me)(OR), CF═CF—P(═O)(CF₃)(OR), CF═CF—P(═O)(Me)(NHR), CF═CF—P(═O)(NHR)(OR), CH═C[P(═O)(OR)]₂]₂, CF═C[P(═O)(OR)₂]₂, CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)]₂]₂, CF [P(═O)(OR)(OR1)]₂, CF[P(═O)(Me)(OR)]₂, CF [P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, and C(OH) [P(═O)(NHR)(OR)]₂;
(iii) SO₃H, SO₂NH₂, SO₂NHTz1, SO₂NHC(═O) (Me, CF₃), SO₂NHC(═O)NH₂, (CRR1)_{q}SO₃H, (CRR1)_{q}SO₂NH₂, (CRR1)_{q}SO₂NHTz1, (CRR1)_{q}SO₂NHC(═O)(Me, CF₃), (CRR1)_{q}SO₂NHC(═O)NH₂, SO₂NHCRR1C(═O)C(═O)OR, SO₂CF₃, CH(SO₂Me)₂, CH(SO₂CF₃)₂, SO₂CRR1C(═O)OR, SO₂CH[C(═O)OR]₂, (CRR1)_{q}SO₂NHCRR1C(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tz1, SO₂(CRR1)_{q}P(═O)(OR)₂, SO₂(CF₂)_{q}C(═O)OR, SO₂(CF₂)_{q}Tz1, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO₂NHC(═O)(Me, CF₃), and C(═NSO₂CF₃)(NHSO₂CF₃);
(iv) NHC(═O)C(═O)OR. NHC(═O)C(═O)O(CRR1)OC(═O)R, NHC(═O)C(═O)O(CRR1)OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTz1, NHC(═O)Tz1, NHSO₂Tz1, NH(CF₂)_{q}Tz1, NHSO₂(CRR1)_{q}C(═O)OR, NHSO₂(CF₂)_{q}C(═O)OR, (CRR1)_{q}NO₂, (CF₂)_{q}NO₂, CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR]CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), and NH(CFR)_{q}P(═O)(CF₃)(OR);
(v) C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂, CH═C[C(═O)OR]₂, CF═C[C(═O)OR]₂, C(R4)═C(R5)(R6), C(═O)C(═O)OR, C(═O)CH[C(═O)OR]₂, C(═O)CH(Tz1)₂, C(═O)CRR1C(═O)(Me, CF₃, Ph), C(═O)CRR1SO₂(Me, CF₃, Ph), (CRR1)_{q}C(═O)C(═O)OR, (CF₂)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, CHFC(═O)CF₃, CHFC(OR)₂CF₃ CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C [C(═O)OH]═NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tz1)₂, C(═O)N[CRR1P(═O)(OR)₂]₂, and C(═O)NHC(CRR1OR)₃;
(vi) Tz1, CR(Tz1)₂, (CRR1)_{q}Tz1, (CF₂)_{q}Tz1, (CFR)_{q}Tz1, CF(Tz1)₂, (CF₂)_{q}CF(Tz1)₂, (CF₂)_{q}CR(Tz1)₂, CR═CR-Tz1, CF═CH-Tz1, CH═CF-Tz1, CF═CF-Tz1, CH═C(Tz1)₂, CF═C(Tz1)₂, or C(H, F)═C(Tz1)[P(═O)(OR)(OR1), P(═O)(Me)(OR), P(═O)(CF₃)(OR), P(═O)(Me)(NHR), P(═O)(NHR)(OR), or C(═O)OR];
(vii) OH, OR, O(CRR1)_{q}C(═O)OR, O(CF₂)_{q}C(═O)OR, OCH[C(═O)OR]₂, O(CRR 1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTz1, O(CRR1)_{q}Tz1, O(CF₂)_{q}Tz1, OCH(Tz1)₂, O(CF₂)_{q}CF(Tz1)₂, O(CF₂)_{q}CR(Tz1)₂, OCF(Tz1)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(NHR)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR 1)_{q}P(═O)(NHR)(OR), O(CRR 1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF [P(═O)(OR)(OR1)]₂, OCF[P(═O)(Me)(OR)]₂, OCF[P(═O)(CF₃)(NHR)]₂, OCF[P(═O)(NHR)(OR)]₂, O(CRR 1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(NHR), O(CRR1)_{q}(CF₂)_{q}P(═O)(NHR)(OR), ON═CH—C(═O)OR, and ON═C[C(═O)OR]CRR1C(═O)OR;
(viii) 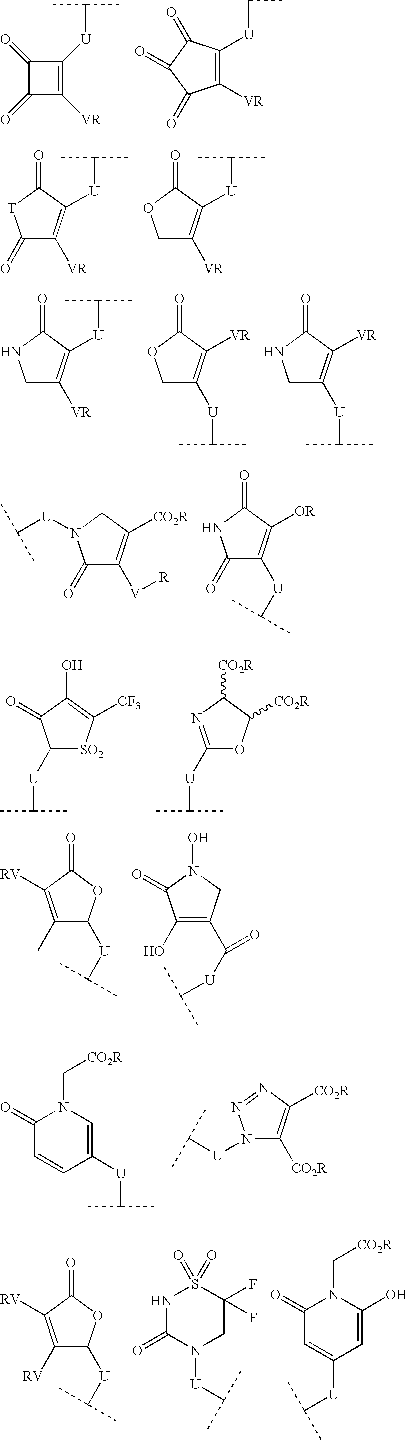 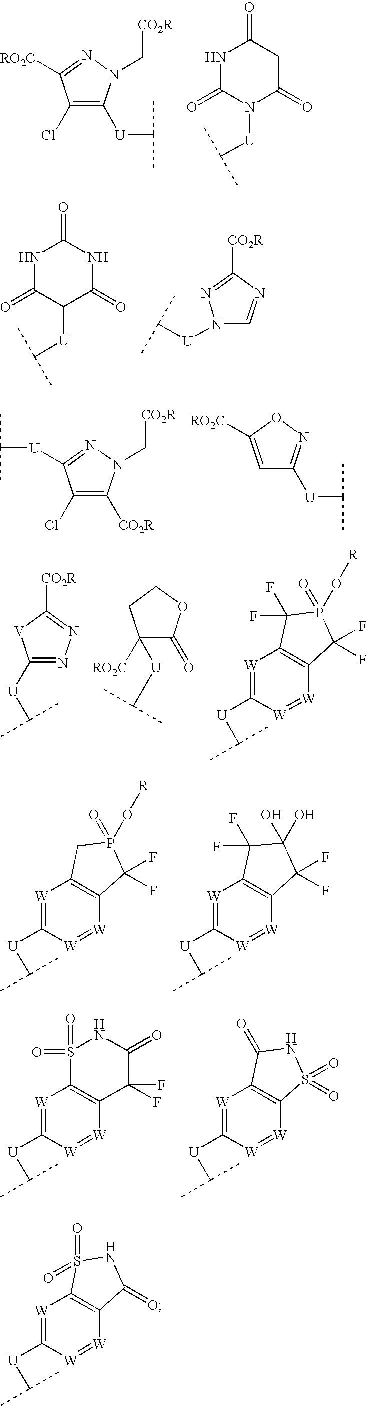
(ix) A₁ through A₆ substituent groups are combined to form stable mono- or bicyclic-fused cycloalkyl, heterocyclyl, heteroaryl, or aryl rings;
wherein:
Y1, Y2, and Y3 are independently selected from (i) or (ii) as follows:
(i) R5, (CR5R6)ₙOR5, OH, (CR5R6)ₙNR5R6, C(═NR5)NR5R6, C(═NOR5)NR5R6, halo, cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5, C(═O)R5, —(CR5R6)ₙC(═O)R5, (CF₂)ₙC(═O)R5, (CFR5)ₙC(═O)R5, tetrazolyl, (CR5R6)ₙTz1, (CF₂)ₙTz1, (CFR5)ₙTz1, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, (CR5R6)ₙC(═O)C(═O)OR5, (CR5R6)ₙCH(OR5)C(═O)OR5, C(R5)═C(R6), C(═O)OR5, C(R5)═C(R6)-Tz1, (CR5R6)ₙP(═O)(OH)₂, (CR5R6)ₙP(═O)(OR5)(OR6), P(═O)(OR5)[(OCR5R6)OC(═O)R5], P(═O)(OR5) [(OCR5R6)OC(═O)OR5], P(═O)[(OCR5R6)OC(═O)R5)][(OCR5R6)OC(═O)R5], P(═O) [(OCR5R6)OC(═O)OR5)] [(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5=CR5—P(═O)(OR5)(OR6), CR5=CR5—P(═O)(Me)(OR5), CC—P(═O)(OR5)(OR6), (C═O)P(═O)(OR5)(OR6), (C═O)P(═O)(Me)(OR5), (C═O)P(═O)(CF₃)(OR5), (CR5OR6)ₙP(═O)(OR5)(OR6), (CR5OR6)ₙP(═O)(Me)(OR5), (CR5OR6)ₙP(═O)(CF₃)(OR5), O(CR5R6)ₙC(═O)OR5, O(CF₂)ₙC(═O)OR5, OCH[C(═O)OR5]₂, O(CR5R6)ₙCH[C(═O)OR5]₂, OCF[C(═O)OR5]₂, O(CR5R6)ₙC(═O)C(═O)OR5, O(CF₂)ₙC(═O)C(═O)OR5, O(CR5R6)ₙTz1, O(CF₂)ₙTz1, OCH(Tz1)₂, O(CF₂)ₙP(═O)(OR5)(OR6), O(CF₂)ₙP(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF[P(═O)(Me)(OR5)]₂, SO₃H, —(CR5R6)ₙSO₃H, S(O)ₙR5, SCF₃, SCHF₂, SO₂CF₃, SO₂Ph, (CR5R6)ₙS(O)ₙR5, (CR5R6)ₙS(O)₂CF₃, (CR5R6)ₙSO₂NR5R6, (CR5R6)ₙSO₂NR5C(═O)(Me, CF₃), (CF₂)ₙSO₃H, (CFR5)ₙSO₃H, and (CF₂)ₙSO₂NR5R6; or
(ii) Y₁ and Y₂, and/or Y₁ and Y₃, and/or Y₂ and Y₃ are selected together to be (CR5R6)₂₋₆, —O[C(R8)(R9)]ᵣO— or —O[C(R8)(R9)]ᵣ₊₁-, where r is an integer from 1 to 4 and R8 and R9 are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroarylalkyl of 5 to 14 ring atoms, and the other of Y1, Y2, and Y3, when not selected as in (ii), is selected as in (i) above;
R and R1 are each independently selected in each case from H, C₁-C₆-alkyl, C₂-C₆-alkenyl and C₂-C₆-alkynyl, where the alkyl, alkenyl or alkynyl group is unsubstituted or mono-, di- or tri-substituted with 1 to 3 substituents selected from the group consisting of Y₁, Y₂, Y₃, an aryl group, —OC(R2R3)OC(═O)R4, and —OC(R2R3)OC(═O)OR4; or R and R1, together with the atoms to which they are attached, form an cycloalkyl, cycloalkenyl, cycloalkynyl, or heterocyclic ring;
where R2, R3 and R4 are independently selected from (i) and (ii) as follows:
(i) H, C₁-C₇ alkyl, alkenyl of 2 to 6 carbon atoms, where the alkenyl group is unsubstituted or mono-, di- or tri-substituted with 1 to 3 substituents selected from the group consisting of Y₁, Y₂, and Y₃, alkynyl of 2 to 6 carbon atoms, where the alkynyl group is unsubstituted or mono-, di- or tri-substituted with 1 to 3 substituents selected from the group consisting of Y₁, Y₂, and Y₃, cycloalkyl of 3 to 8 carbon atoms, where the cycloalkyl group is unsubstituted or mono-, di- or tri-substituted with 1 to 3 substituents selected from the group consisting of Y₁, Y₂, and Y₃, aryl of 6 to 14 carbon atoms, where the aryl group is unsubstituted or mono-, di- or tri-substituted with 1 to 3 substituents selected from the group consisting of Y₁, Y₂, and Y₃, linked biaryl or heterobiaryl groups of 10 to 20 atoms featuring two aromatic or heteroaromatic ring systems linked through a single bond, with the ring atoms selected from carbon and heteroatoms, where the heteroatoms are selected from oxygen, nitrogen, and sulfur, and where the linked biaryl or heterobiaryl group is unsubstituted or mono-, di- or tri-substituted with 1 to 3 substituents selected from the group consisting of Y₁, Y₂, and Y₃, aralkyl of 7 to 16 carbon atoms, where the aralkyl is unsubstituted or mono-, di- or tri-substituted with 1 to 3 substituents selected from the group consisting of Y₁, Y₂, and Y₃, monocyclic-heteroaryl or bicyclic-heteroaryl having 5 to 14 ring atoms with the ring atoms selected from carbon and heteroatoms, where the heteroatoms are selected from oxygen, nitrogen, and sulfur, and where the monocyclic-heteroaryl or bicyclic heteroaryl group is unsubstituted or mono-, di- or tri-substituted with 1 to 3 substituents selected from the group consisting of Y₁, Y₂, and Y₃, and a heteroaralkyl group of 5 to 14 ring atoms with the ring atoms selected from carbon and heteroatoms, where the heteroatoms are selected from oxygen, nitrogen, and sulfur, where the heteroaralkyl is unsubstituted or substituted on the alkyl chain and which is unsubstituted on the ring or mono-, di- or tri-substituted on the ring with 1 to 3 substituents selected from the group consisting of Y₁, Y₂, and Y₃; or
(ii) R2 and R3, and/or R3 and R4, and/or R2 and R4 are joined to form a 4-8-membered cycloalkyl, cycloalkenyl, cycloalkynyl, or heterocyclic ring, and the other of R2, R3, and R4, when not joined in a ring, is selected as in (i) above;
R5 and R6 are each independently H, C1-C6 alkyl, C2-C6 alkenyl, C2-C6 alkynyl, a C3-C8 cycloalkyl ring, or a 5-7 membered heterocyclic ring;
T is O, NR1 or CR;
U and V are each independently selected from a direct link, (CRR1)_{q}, O, S, NR1; and
W is CR or N;
each n is, independently, 0 to 2; and
each q is, independently, 1 to 3
and prodrugs thereof.

91. The compound of claim 90, wherein
L₁ is a bond, CH₂, CH₂CH₂, CRR₁, or C(RR₁)C(RR₁),
where R and R1 are independently selected from hydrogen, an alkyl group of 1 to 6 carbon atoms, where the alkyl group is unsubstituted or mono-, di- or tri-substituted with 1 to 3 substituents selected from the group consisting of Y₁, Y₂, Y₃, an aryl group, —OC(R2R3)OC(═O)R4, and —OC(R2R3)OC(═O)OR4, or where R and R1 are joined to form a 4-8 membered cycloalkyl, cycloalkenyl, cycloalkynyl, or heterocyclic ring.

92. The compound of claim 90, wherein L₁ is CH₂CH₂ or CH₂.

93. The compound of claim 92, wherein L₁ is CH₂.

94. The compound of claim 90, wherein X₁ and X₂ are CR₇ or N, where R₇ is H, C₁-C₃ alkyl, fluoro, chloro or bromo.

95. The compound of claim 90, wherein X₁ and X₂ are each independently CG₂ or CG₃, where G₂ is H, C₁-C₃ alkyl, phenyl, C₁₋₃alkylenephenyl or heteroaryl, where the phenyl, C₁₋₃alkylenephenyl or heteroaryl ring is optionally substituted with (i)-(iv) as follows:
(i) F, Cl, Br, CN, CF₃, OR, carboxy, alkylenedioxy, alkylene where the alkylene group forms a fused bicyclic group with the phenyl, C₁₋₃alkylenephenyl or heteroaryl ring, (CRR₁)ₙCO₂R, CF₂CO₂R, O(CRR₁)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR₁)CO₂R, C₁-C₃-alkylsulfonyl, or CF₂P(═O)(OR)(OR1);
(ii) phenyl, where the phenyl is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1);
(iii) phenoxy, where the phenoxy is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1); or
(iv) benzyloxy, where the benzyloxy is optionally further substituted with F, Cl, Br, CF₃, OR, methoxycarbonyl, carboxy, (CRR1)ₙCO₂R, CF₂CO₂R, O(CRR1)ₙCO₂R, CH═CHCO₂R, tetrazolyl (Tz1), NRR1, NRC(═O)OR1, OC(═O)NRR1, C(═O)NRR1, NRC(═O)C(═O)OR1, SO₂NRR1, S(O)ₘ(CRR1)ₙCO₂R, SO₂NRR1, C₁-C₃-alkylsulfonyl, or CF₂P(═O)(OR)(OR1); and G₃ is H, C₁-C₃ alkyl, C₁-C₃ alkoxy, Cl, F, Br, or a phenyl, benzyl or pyridyl ring, where the phenyl, benzyl or pyridyl ring is optionally and independently substituted with 1, 2, or 3 of the following moieties: Cl, F, Br, CN, carboxy, (CRR1)ₙCO₂R, OCF₃, OCHF₂, C₁-C₃ alkyl, and C₁-C₃-alkylsulfonyl;
where each m is independently 0 to 6 and each n is independently 0 to 2.

96. The compound of claim 90, wherein G₁ is a phenyl ring, where the phenyl ring is substituted with one or more substituents selected from the group consisting of phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester, where acyl is C₂-C₇ alkanoyl or C4-C7 cycloalkanoyl, and phosphonodifluoromethyl mono-alkoxycarbonyloxymethyl ester, where alkoxy is C1-C6 or C3-C6 cycloalkoxy;
and is optionally further substituted with one or more groups selected from 2-carboxyethenyl optionally substituted with 1-2 fluorines or methyl groups, carboxymethoxy, carboxy-C2-C4-alkyl optionally further substituted with 1-4 halogen atoms or 1-4 methyl groups, Cl, Br, F, I, CN, OH, CH₃, and ethynyl.

97. The compound of claim 90, wherein G₃ and G₄ are linked together to form an alicyclic, aromatic or aromatic ring.

98. The compound of claim 90, wherein G₂ and G₃ are linked together to form an alicyclic, aromatic or aromatic ring.

99. The compound of claim 90, wherein the compound has formula II:

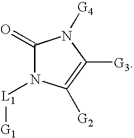

100. The compound of claim 90, wherein the compound is:
3-[3-Bromo-4-(difluoro-phosphono-methyl)-benzyl]-4-(4-methanesulfonyl-phenyl)-2-oxo-2,3-dihydro-imidazole-1-carboxylic acid ethyl ester;
4-{1-Benzyl-3-[3-bromo-4-(difluoro-phosphono-methyl)-benzyl]-2-oxo-2,3-dihydro-1H-imidazol-4-yl}-benzoic acid;
({2-Bromo-4-[5-(4-methanesulfonyl-phenyl)-2-oxo-2,3-dihydro-imidazol-1-ylmethyl]-phenyl}-difluoro-methyl)-phosphonic acid;
({2-Bromo-4-[5-(4-methanesulfonyl-phenyl)-3-methyl-2-oxo-2,3-dihydro-imidazol-1-ylmethyl]-phenyl}-difluoro-methyl)-phosphonic acid;
4-{1-Benzyl-3-[3-bromo-4-(difluoro-phosphono-methyl)-benzyl]-2-oxo-2,3-dihydro-1H-imidazol-4-yl}-benzoic acid methyl ester;
4-{3-[3-Bromo-4-(difluoro-phosphono-methyl)-benzyl]-2-oxo-2,3-dihydro-1H-imidazol-4-yl}-benzoic acid;
4-{1-Benzyl-3-[3-chloro-4-(difluoro-phosphono-methyl)-benzyl]-2-oxo-2,3-dihydro-1H-imidazol-4-yl}-benzoic acid;
4-{1-Benzyl-3-[3-chloro-4-(difluoro-phosphono-methyl)-benzyl]-2-oxo-2,3-dihydro-1H-imidazol-4-yl}-benzoic acid methyl ester;
4-[3-[3-Bromo-4-(difluoro-phosphono-methyl)-benzyl]-1-(4-fluoro-benzyl)-2-oxo-2,3-dihydro-1H-imidazol-4-yl]-benzoic acid methyl ester;
4-{3-[3-Bromo-4-(difluoro-phosphono-methyl)-benzyl]-2-oxo-1-phenethyl-2,3-dihydro-1H-imidazol-4-yl}-benzoic acid methyl ester;
4-[3-[3-Bromo-4-(difluoro-phosphono-methyl)-benzyl]-1-(4-fluoro-benzyl)-2-oxo-2,3-dihydro-1H-imidazol-4-yl]-benzoic acid;
4-{3-[3-Bromo-4-(difluoro-phosphono-methyl)-benzyl]-2-oxo-1-phenethyl-2,3-dihydro-1H-imidazol-4-yl}-benzoic acid;
4-[3-[3-Bromo-4-(difluoro-phosphono-methyl)-benzyl]-1-(4-cyano-benzyl)-2-oxo-2,3-dihydro-1H-imidazol-4-yl]-benzoic acid methyl ester;
4-{1-Benzyl-3-[3-bromo-4-(difluoro-phosphono-methyl)-benzyl]-2-oxo-2,3-dihydro-1H-imidazol-4-yl}-benzoic acid ethyl ester;
({4-[3-Benzyl-5-(4-methanesulfonyl-phenyl)-2-oxo-2,3-dihydro-imidazol-1-ylmethyl]-2-bromo-phenyl}-difluoro-methyl)-phosphonic acid;
4-{3-[3-Bromo-4-(difluoro-phosphono-methyl)-benzyl]-2-oxo-1-phenyl-2,3-dihydro-1H-imidazol-4-yl}-benzoic acid methyl ester;
4-{3-[3-Bromo-4-(difluoro-phosphono-methyl)-benzyl]-2-oxo-1-phenyl-2,3-dihydro-1H-imidazol-4-yl}-benzoic acid;
({4-[3-Benzyl-5-(4-cyano-phenyl)-2-oxo-2,3-dihydro-imidazol-1-ylmethyl]-2-bromo-phenyl}-difluoro-methyl)-phosphonic acid;
({2-Bromo-4-[3-cyclopropyl-5-(4-methanesulfonyl-phenyl)-2-oxo-2,3-dihydro-imidazol-1-ylmethyl]-phenyl}-difluoro-methyl)-phosphonic acid;
({2-Bromo-4-[3-(3-fluoro-benzyl)-5-(4-methanesulfonyl-phenyl)-2-oxo-2,3-dihydro-imidazol-1-ylmethyl]-phenyl}-difluoro-methyl)-phosphonic acid;
4-[3-[3-Bromo-4-(difluoro-phosphono-methyl)-benzyl]-1-(3,5-dimethoxy-benzyl)-2-oxo-2,3-dihydro-1H-imidazol-4-yl]-benzoic acid methyl ester;
4-{3-[3-Bromo-4-(difluoro-phosphono-methyl)-benzyl]-1-cyclopropyl-2-oxo-2,3-dihydro-1H-imidazol-4-yl}-benzoic acid methyl ester;
4-{3-[3-Bromo-4-(difluoro-phosphono-methyl)-benzyl]-2-oxo-1-pyridin-2-ylmethyl-2,3-dihydro-1H-imidazol-4-yl}-benzoic acid methyl ester;
({4-[3-Benzyl-5-(4-chloro-phenyl)-2-oxo-2,3-dihydro-imidazol-1-ylmethyl]-2-bromo-phenyl}-difluoro-methyl)-phosphonic acid;
({4-[3-Benzyl-5-(4-carbamoyl-phenyl)-2-oxo-2,3-dihydro-imidazol-1-ylmethyl]-2-bromo-phenyl}-difluoro-methyl)-phosphonic acid;
({4-[3-Benzyl-5-(4-methylcarbamoyl-phenyl)-2-oxo-2,3-dihydro-imidazol-1-ylmethyl]-2-bromo-phenyl}-difluoro-methyl)-phosphonic acid;
{[4-(3-Benzyl-2-oxo-5-phenyl-2,3-dihydro-imidazol-1-ylmethyl)-2-bromo-phenyl]-difluoro-methyl}-phosphonic acid;
({4-[3-Benzyl-5-(2,3-dihydro-benzo[1,4]dioxin-6-yl)-2-oxo-2,3-dihydro-imidazol-1-ylmethyl]-2-bromo-phenyl}-difluoro-methyl)-phosphonic acid;
({4-[3-Benzyl-5-(3-cyano-phenyl)-2-oxo-2,3-dihydro-imidazol-1-ylmethyl]-2-bromo-phenyl}-difluoro-methyl)-phosphonic acid;
({4-[3-(3-Amino-benzyl)-5-(4-methanesulfonyl-phenyl)-2-oxo-2,3-dihydro-imidazol-1-ylmethyl]-2-bromo-phenyl}-difluoro-methyl)-phosphonic acid;
{[2-Bromo-4-(3-cyclopropylmethyl-2-oxo-5-phenyl-2,3-dihydro-imidazol-1-ylmethyl)-phenyl]-difluoro-methyl}-phosphonic acid;
4-[3-[3-Bromo-4-(difluoro-phosphono-methyl)-benzyl]-1-(3,5-dimethoxy-benzyl)-2-oxo-2,3-dihydro-1H-imidazol-4-yl]-benzoic acid;
4-{3-[3-Bromo-4-(difluoro-phosphono-methyl)-benzyl]-1-cyclopropyl-2-oxo-2,3-dihydro-1H-imidazol-4-yl}-benzoic acid;
4-{3-[3-Bromo-4-(difluoro-phosphono-methyl)-benzyl]-2-oxo-1-pyridin-2-ylmethyl-2,3-dihydro-1H-imidazol-4-yl}-benzoic acid.
3-[3-Bromo-4-(difluoro-phosphono-methyl)-benzyl]-4-(4-methoxycarbonyl-phenyl)-2-oxo-2,3-dihydro-imidazole-1-carboxylic acid ethyl ester;
4-{3-[3-Bromo-4-(difluoro-phosphono-methyl)-benzyl]-1-methyl-2-oxo-2,3-dihydro-1H-imidazol-4-yl}-benzoic acid methyl ester;
{[4-(3-Benzyl-2-oxo-5-thiophen-3-yl-2,3-dihydro-imidazol-1-ylmethyl)-2-bromo-phenyl]-difluoro-methyl}-phosphonic acid;
({4-[3-(3-Acetylamino-benzyl)-5-(4-methanesulfonyl-phenyl)-2-oxo-2,3-dihydro-imidazol-1-ylmethyl]-2-bromo-phenyl}-difluoro-methyl)-phosphonic acid;
4-{3-[3-Bromo-4-(difluoro-phosphono-methyl)-benzyl]-2-oxo-2,3-dihydro-1H-imidazol-4-yl}-benzoic acid methyl ester;
4-{3-[3-Bromo-4-(difluoro-phosphono-methyl)-benzyl]-1-methyl-2-oxo-2,3-dihydro-1H-imidazol-4-yl}-benzoic acid;
{[2-Bromo-4-(2-oxo-3-phenyl-2,3-dihydro-imidazol-1-ylmethyl)-phenyl]-difluoro-methyl}-phosphonic acid;
{[4-(3-Benzyl-2-oxo-5-thiophen-2-yl-2,3-dihydro-imidazol-1-ylmethyl)-2-bromo-phenyl]-difluoro-methyl}-phosphonic acid;
3-(4-{3-[3-Bromo-4-(difluoro-phosphono-methyl)-benzyl]-1-cyclopropyl-2-oxo-2,3-dihydro-1H-imidazol-4-yl}-phenyl)-acrylic acid methyl ester;
3-(4-{3-[3-Bromo-4-(difluoro-phosphono-methyl)-benzyl]-1-cyclopropyl-2-oxo-2,3-dihydro-1H-imidazol-4-yl}-phenyl)-acrylic acid;
4-{3-[3-Bromo-4-(difluoro-phosphono-methyl)-benzyl]-1-ethyl-2-oxo-2,3-dihydro-1H-imidazol-4-yl}-benzoic acid methyl ester;
4-{3-[3-Bromo-4-(difluoro-phosphono-methyl)-benzyl]-2-oxo-1-propyl-2,3-dihydro-1H-imidazol-4-yl}-benzoic acid methyl ester;
({4-[3-Benzyl-2-oxo-5-(5,6,7,8-tetrahydro-naphthalen-2-yl)-2,3-dihydro-imidazol-1-ylmethyl]-2-bromo-phenyl}-difluoro-methyl)-phosphonic acid;
({2-Bromo-4-[3-(4-methanesulfonyl-benzyl)-2-oxo-2,3-dihydro-imidazol-1-ylmethyl]-phenyl}-difluoro-methyl)-phosphonic acid;
(4-{3-[3-Bromo-4-(difluoro-phosphono-methyl)-benzyl]-1-cyclopropyl-2-oxo-2,3-dihydro-1H-imidazol-4-yl}-phenoxy)-acetic acid methyl ester;
(4-{3-[3-Bromo-4-(difluoro-phosphono-methyl)-benzyl]-1-cyclopropyl-2-oxo-2,3-dihydro-1H-imidazol-4-yl}-phenoxy)-acetic acid;
4-[1-Benzyl-3-(3-bromo-4-{[(2,2-dimethyl-propionyloxymethoxy)-hydroxy-phosphoryl]-difluoro-methyl}-benzyl)-2-oxo-2,3-dihydro-1H-imidazol-4-yl]-benzoic acid 2,2-dimethyl-propionyloxymethyl ester;
4-{3-[3-Bromo-4-(difluoro-phosphono-methyl)-benzyl]-2-oxo-4-phenyl-2,3-dihydro-imidazol-1-ylmethyl}-benzoic acid methyl ester;
4-{3-[3-Bromo-4-(difluoro-phosphono-methyl)-benzyl]-2-oxo-4-phenyl-2,3-dihydro-imidazol-1-ylmethyl}-benzoic acid;
2,2-Dimethyl-propionic acid ({4-[3-benzyl-5-(4-cyano-phenyl)-2-oxo-2,3-dihydro-imidazol-1-ylmethyl]-2-bromo-phenyl}-difluoro-methyl)-hydroxy-phosphinoyloxymethyl ester;
2,2-Dimethyl-propionic acid ({4-[3-benzyl-5-(4-cyano-phenyl)-2-oxo-2,3-dihydro-imidazol-1-ylmethyl]-2-bromo-phenyl}-difluoro-methyl)-(2,2-dimethyl-propionyloxymethoxy)-phosphinoyloxymethyl ester;
({2-Bromo-4-[3-(4-methanesulfonyl-benzyl)-2-oxo-5-phenyl-2,3-dihydro-imidazol-1-ylmethyl]-phenyl}-difluoro-methyl)-phosphonic acid;
4-[1-Benzyl-3-(3-bromo-4-{difluoro-[hydroxy-(1-isopropoxycarbonyloxy-ethoxy)-phosphoryl]-methyl}-benzyl)-2-oxo-2,3-dihydro-1H-imidazol-4-yl]-benzoic acid 1-isopropoxycarbonyloxy-ethyl ester;
({2-Bromo-4-[3-(3-methoxy-benzyl)-2-oxo-2,3-dihydro-imidazol-1-ylmethyl]-phenyl}-difluoro-methyl)-phosphonic acid;
4-{3-[3-Bromo-4-(difluoro-phosphono-methyl)-benzyl]-2-oxo-2,3-dihydro-imidazol-1-yl}-benzoic acid ethyl ester;
3-{3-[3-Bromo-4-(difluoro-phosphono-methyl)-benzyl]-1-cyclopropyl-2-oxo-2,3-dihydro-1H-imidazol-4-yl}-benzoic acid methyl ester;
3-{3-[3-Bromo-4-(difluoro-phosphono-methyl)-benzyl]-1-cyclopropyl-2-oxo-2,3-dihydro-1H-imidazol-4-yl}-benzoic acid;
2,2-Dimethyl-propionic acid ({2-bromo-4-[5-(4-methanesulfonyl-phenyl)-2-oxo-2,3-dihydro-imidazol-1-ylmethyl]-phenyl}-difluoro-methyl)-hydroxy-phosphinoyloxymethyl ester;
({2-Bromo-4-[5-(4-methanesulfonyl-phenyl)-2-oxo-2,3-dihydro-imidazol-1-ylmethyl]-phenyl}-difluoro-methyl)-phosphonic acid mono-(1-isopropoxycarbonyloxy-ethyl) ester;
3-(3-Bromo-4-{[(2,2-dimethyl-propionyloxymethoxy}hydroxy-phosphoryl]-difluoro-methyl-benzyl)-4-(4-methoxycarbonyl-phenyl)-2-oxo-2,3-dihydro-imidazole-1-carboxylic acid ethyl ester;
4-{3-Benzyl-1-[3-bromo-4-(difluoro-phosphono-methyl)-benzyl]-2-oxo-2,3-dihydro-1H-imidazol-4-yl}-benzoic acid methyl ester;
4-{3-Benzyl-1-[3-bromo-4-(difluoro-phosphono-methyl)-benzyl]-2-oxo-2,3-dihydro-1H-imidazol-4-yl}-benzoic acid;
2,2-Dimethyl-propionic acid ({2-bromo-4-[5-(4-methanesulfonyl-phenyl)-3-methyl-2-oxo-2,3-dihydro-imidazol-1-ylmethyl]-phenyl}-difluoro-methyl)-hydroxy-phosphinoyloxymethyl ester;
({2-Bromo-4-[5-(4-methanesulfonyl-phenyl)-3-methyl-2-oxo-2,3-dihydro-imidazol-1-ylmethyl]-phenyl}-difluoro-methyl)-phosphonic acid mono-(1-isopropoxycarbonyloxy-ethyl) ester;
{[4-(3-Benzyl-2-oxo-2,3-dihydro-imidazol-1-ylmethyl)-2-bromo-phenyl]-difluoro-methyl}-phosphonic acid;
({4-[3-Benzyl-5-(4-chloro-phenyl)-2-oxo-2,3-dihydro-imidazol-1-ylmethyl]-2-bromo-phenyl}-difluoro-methyl)-phosphonic acid mono-(1-isopropoxycarbonyloxy-ethyl) ester;
2-(4-{3-[3-Bromo-4-(difluoro-phosphono-methyl)-benzyl]-1-cyclopropyl-2-oxo-2,3-dihydro-1H-imidazol-4-yl}-phenoxy)-2-methyl-propionic acid ethyl ester;
2-(4-{3-[3-Bromo-4-(difluoro-phosphono-methyl)-benzyl]-1-cyclopropyl-2-oxo-2,3-dihydro-1H-imidazol-4-yl}-phenoxy)-2-methyl-propionic acid;
3-[3-Bromo-4-(difluoro-phosphono-methyl)-benzyl]-4-(4-cyano-phenyl)-2-oxo-2,3-dihydro-imidazole-1-carboxylic acid ethyl ester;
2,2-Dimethyl-propionic acid ({4-[3-benzyl-5-(4-methanesulfonyl-phenyl)-2-oxo-2,3-dihydro-imidazol-1-ylmethyl]-2-bromo-phenyl}-difluoro-methyl)-hydroxy-phosphinoyloxymethyl ester;
2,2-Dimethyl-propionic acid 2-{4-[3-(3-bromo-4-{[(2,2-dimethyl-propionyloxymethoxy)-hydroxy-phosphoryl]-difluoro-methyl}-benzyl)-1-cyclopropyl-2-oxo-2,3-dihydro-1H-imidazol-4-yl]-phenoxy}-acetoxymethyl ester;
({2-Bromo-4-[5-(4-cyano-phenyl)-2-oxo-2,3-dihydro-imidazol-1-ylmethyl]-phenyl}-difluoro-methyl)-phosphonic acid;
({4-[3-Benzyl-5-(4-methanesulfonyl-phenyl)-2-oxo-2,3-dihydro-imidazol-1-ylmethyl]-2-bromo-phenyl}-difluoro-methyl)-phosphonic acid mono-(1-isopropoxycarbonyloxy-ethyl)ester;
4-[3-(3-Bromo-4-{[(2,2-dimethyl-propionyloxymethoxy)-hydroxy-phosphoryl]-difluoro-methyl}-benzyl)-1-methyl-2-oxo-2,3-dihydro-1H-imidazol-4-yl]-benzoic acid 2,2-dimethyl-propionyloxymethyl ester;
4-{3-[3-Bromo-4-(difluoro-phosphono-methyl)-benzyl]-2-oxo-2,3-dihydro-imidazol-1-ylmethyl}-benzoic acid methyl ester;
(4-{3-[3-Bromo-4-(difluoro-phosphono-methyl)-benzyl]-2-oxo-2,3-dihydro-imidazol-1-ylmethyl}-2-chloro-phenoxy)-acetic acid methyl ester;
(4-{3-[3-Bromo-4-(difluoro-phosphono-methyl)-benzyl]-2-oxo-2,3-dihydro-imidazol-1-ylmethyl}-2-chloro-phenoxy)-acetic acid;
({2-Bromo-4-[5-(4-methanesulfonyl-phenyl)-2-oxo-2,3-dihydro-imidazol-1-ylmethyl]-phenyl}-difluoro-methyl)-phosphonic acid mono-(3-hexadecyloxy-propyl)ester;
4-{3-[3-Bromo-4-(difluoro-phosphono-methyl)-benzyl]-2-oxo-2,3-dihydro-imidazol-1-ylmethyl}-benzoic acid;
4-{3-[3-Bromo-4-(difluoro-phosphono-methyl)-benzyl]-2-oxo-2,3-dihydro-benzoimidazol-1-ylmethyl}-benzoic acid methyl ester;
{[2-Bromo-4-(2-oxo-2,3-dihydro-imidazol-1-ylmethyl)-phenyl]-difluoro-methyl}-phosphonic acid;
[(2-Bromo-4-{3-[3-bromo-4-(difluoro-phosphono-methyl)-benzyl]-2-oxo-2,3-dihydro-imidazol-1-ylmethyl}-phenyl)-difluoro-methyl]-phosphonic acid; or
{[4-(3-Benzyl-2-oxo-imidazolidin-1-ylmethyl)-2-bromo-phenyl]-difluoro-methyl}-phosphonic acid.

101. The compound of claim 90, wherein said compound is a prodrug that has the formula ArCF₂P(O)(OH)(OCH(H/Me)OC(═O)OiPr, ArCF₂P(O)[(OCH(H/Me)OC(═O)OiPr]₂, ArCF₂P(O)(OH)(OCH(H/Me)OC(═O)tBu, or ArCF2P(O)[(OCH(H/Me)OC(═O)tBu]₂.

102. The compound of claim 90, wherein said compound is a prodrug that is a mono- or bis-amidate prodrug, a mono- or di-lipid ester prodrug, a mono- or di-alpha-acyloxyalkyl ester or amide prodrug, a cytochrome P450 3A activated prodrug, a cyclic diester prodrug, a cyclic monoester monoamide prodrug, a cyclic diamide prodrug, or a carbohydrate prodrug.

103. The compound of claim 101, wherein said prodrug has the formula ROCH₂CHR′CH₂O—P(O)(OH)CF₂Ar or (ROCH₂CHR′CH₂O)₂—P(O)CF₂Ar, where R is C₁₄₋₂₀-n-alkyl and R′ is H, OH or OMe.

104. A pharmaceutical composition comprising the compound of claim 90 and a pharmaceutically acceptable carrier.

105. The pharmaceutical compositon according to claim 104, further comprising one or more of the following agents: antiobesity agents, antidiabetics, antihypertensive agents, agents for the treatment and/or prevention of complications resulting from or associated with diabetes apetite regulating agents, and agents for the treatment and/or prevention of complications and disorders resulting from or associated with obesity.

106. The pharmaceutical composition according to claim 105, wherein said one or more of the following agents are selected from: CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators, TR B agonists, leptin, dexamphetamine or amphetamine, fenfluramine or dexfenfluramine, sibutramine, orlistat, mazindol, phentermine, insulin, GLP-1 (glucagons like peptide-1) or derivatives thereof, orally active hypoglycemic agents, sulfonylureas, biguanides, meglitinides, oxadiazolidinediones, thizolidinediones, glucosidase inhibitors, glucagon antagonists, GLP-1 agonists, potassium channel openers, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism, antihyperlipidemic agents, antilipedimic agents as HMG CoA inhibitors (statins), compounds lowering food intake, PPAR and RXR agonists, agents acting on the ATP-dependent potassium channel of the B-cells, tolbutamide, glibenclamide, glipizide or glicazide, metformin, asrepaglinide, troglitazone, ciglitazone, pioglitazone, rosiglitazone, 5-[[4-[3-Methyl-4-oxo-3,4-dihydro-2-quinazolinyl]methoxy]phenyl-methyl]thiazolidine-2,4-dione or a pharmaceutically acceptable salt thereof, (−) 3-[4-[2-phenoxazin-10-yl ethoxy]phenyl]-2-ethoxypropanoic acid or a pharmaceutically acceptable salt thereof, miglitol, acarbose, tolbutamide, glibenclamide, glipizide, gliclazide, repaglinide, nateglinide, cholestyramine, colestipol, clofibrate, gemfibrozil, lovastatin, pravastatin, simvastatin, probucol, dextrothyroxine, alprenolol, atenolol, timolot, pindolol, propranolol and metoprolol, benazepril, captopril, analapril, fosinopril, lisinopril, quinapril, ramipril, nifedipine, felodipine, nicardipine, isradipine, nimodipine, diltiazem, verapamil, doxazosin, urapidil, prazosin or terazosin.

107. The pharmaceutical composition of claim 105, further comprising one or more of the following: a sulphonylurea and metformin, a sulphonylurea and acarbose, repaglinide and metformin, insulin and a sulphonylurea, insulin and metformin, insulin and lovastatin or CART agonist and a CCK agonist.

108. A method of modulating the activity of a protein tyrosine phosphatase comprising contacting the protein tyrosine phosphatase with a compound of claim 90 or a pharmaceutically acceptable compositon thereof.

109. The method of claim 108, wherein the protein tyrosine phosphatase is protein tyrosine phosphatase 1B.

110. The method of claim 108, wherein the protein tyrosine phosphatase is inhibited by the compound or composition.

111. The method of claim 108, wherein said pharmaceutically acceptable compositon further comprises one or more of the following agents: antiobesity agents, antidiabetics, antihypertensive agents, agents for the treatment and/or prevention of complications resulting from or associated with diabetes apetite regulating agents, and agents for the treatment and/or prevention of complications and disorders resulting from or associated with obesity.

112. The method of claim 111, wherein said one or more of the following agents are selected from: CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors. PPAR (peroxisome proliferator activated receptor) modulators, RXR (retinoid X receptor) modulators, TR B agonists, leptin, dexamphetamine or amphetamine, fenfluramine or dexfenfluramine, sibutramine, orlistat, mazindol, phentermine, insulin, GLP-1 (glucagons like peptide-1) or derivatives thereof, orally active hypoglycemic agents, sulfonylureas, biguanides, meglitinides, oxadiazolidinediones, thizolidinediones, glucosidase inhibitors, glucagon antagonists. GLP-1 agonists, potassium channel openers, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism, antihyperlipidemic agents, antilipedimic agents as HMG CoA inhibitors (statins), compounds lowering food intake, PPAR and RXR agonists, agents acting on the ATP-dependent potassium channel of the B-cells, tolbutamide, glibenclamide, glipizide or glicazide, metformin, asrepaglinide, troglitazone, ciglitazone, pioglitazone, rosiglitazone, 5-[[4-[3-Methyl-4-oxo-3,4-dihydro-2-quinazolinyl]methoxy]phenyl-methyl]thiazolidine-2,4-dione or a pharmaceutically acceptable salt thereof, (−) 3-[4-[2-phenoxazin-10-yl ethoxy]phenyl]-2-ethoxypropanoic acid or a pharmaceutically acceptable salt thereof, miglitol, acarbose, tolbutamide, glibenclamide, glipizide, gliclazide, repaglinide, nateglinide, cholestyramine, colestipol, clofibrate, gemfibrozil, lovastatin, pravastatin, simvastatin, probucol, dextrothyroxine, alprenolol, atenolol, timolot, pindolol, propranolol and metoprolol, benazepril, captopril, analapril, fosinopril, lisinopril, quinapril, ramipril, nifedipine, felodipine, nicardipine, isradipine, nimodipine, diltiazem, verapamil, doxazosin, urapidil, prazosin or terazosin.

113. The method of claim 108, wherein said pharmaceutically acceptable composition further comprises one or more of the following: a sulphonylurea and metformin, a sulphonylurea and acarbose, repaglinide and metformin, insulin and a sulphonylurea, insulin and metformin, insulin and lovastatin or CART agonist and a CCK agonist.

114. A method of increasing insulin sensitivity comprising administering a compound of claim 90 or a pharmaceutically acceptable compositon thereof.

115. The method of claim 114, wherein said pharmaceutically acceptable compositon further comprises one or more of the following agents: antiobesity agents, antidiabetics, antihypertensive agents, agents for the treatment and/or prevention of complications resulting from or associated with diabetes apetite regulating agents, and agents for the treatment and/or prevention of complications and disorders resulting from or associated with obesity.

116. The method of claim 115, wherein said one or more of the following agents are selected from: CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators, TR B agonists, leptin, dexamphetamine or amphetamine, fenfluramine or dexfenfluramine, sibutramine, orlistat, mazindol, phentermine, insulin, GLP-1 (glucagons like peptide-1) or derivatives thereof, orally active hypoglycemic agents, sulfonylureas, biguanides, meglitinides, oxadiazolidinediones, thizolidinediones, glucosidase inhibitors, glucagon antagonists, GLP-1 agonists, potassium channel openers, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism, antihyperlipidemic agents, antilipedimic agents as HMG CoA inhibitors (statins), compounds lowering food intake, PPAR and RXR agonists, agents acting on the ATP-dependent potassium channel of the B-cells, tolbutamide, glibenclamide, glipizide or glicazide, metformin, asrepaglinide, troglitazone, ciglitazone, pioglitazone, rosiglitazone, 5-[[4-[3-Methyl-4-oxo-3,4-dihydro-2-quinazolinyl]methoxy]phenyl-methyl]thiazolidine-2,4-dione or a pharmaceutically acceptable salt thereof, (−) 3-[4-[2-phenoxazin-10-yl ethoxy]phenyl]-2-ethoxypropanoic acid or a pharmaceutically acceptable salt thereof, miglitol, acarbose, tolbutamide, glibenclamide, glipizide, gliclazide, repaglinide, nateglinide, cholestyramine, colestipol, clofibrate, gemfibrozil, lovastatin, pravastatin, simvastatin, probucol, dextrothyroxine, alprenolol, atenolol, timolot, pindolol, propranolol and metoprolol, benazepril, captopril, analapril, fosinopril, lisinopril, quinapril, ramipril, nifedipine, felodipine, nicardipine, isradipine, nimodipine, diltiazem, verapamil, doxazosin, urapidil, prazosin or terazosin.

117. The method of claim 114, wherein said pharmaceutically acceptable composition further comprises one or more of the following: a sulphonylurea and metformin, a sulphonylurea and acarbose, repaglinide and metformin, insulin and a sulphonylurea, insulin and metformin, insulin and lovastatin or CART agonist and a CCK agonist.

118. A method of treating, preventing, or ameliorating one or more symptoms of a protein tyrosine phosphatase mediated disease comprising administering a compound of claim 90, or a pharmaceutically acceptable composition thereof, to an individual.

119. The method of claim 118, wherein said pharmaceutically acceptable compositon further comprises one or more of the following agents: antiobesity agents, antidiabetics, antihypertensive agents, agents for the treatment and/or prevention of complications resulting from or associated with diabetes apetite regulating agents, and agents for the treatment and/or prevention of complications and disorders resulting from or associated with obesity.

120. The method of claim 119, wherein said one or more of the following agents are selected from: CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators, TR B agonists, leptin, dexamphetamine or amphetamine, fenfluramine or dexfenfluramine, sibutramine, orlistat, mazindol, phentermine, insulin, GLP-1 (glucagons like peptide-1) or derivatives thereof, orally active hypoglycemic agents, sulfonylureas, biguanides, meglitinides, oxadiazolidinediones, thizolidinediones, glucosidase inhibitors, glucagon antagonists, GLP-1 agonists, potassium channel openers, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism, antihyperlipidemic agents, antilipedimic agents as HMG CoA inhibitors (statins), compounds lowering food intake, PPAR and RXR agonists, agents acting on the ATP-dependent potassium channel of the B-cells, tolbutamide, glibenclamide, glipizide or glicazide, metformin, asrepaglinide, troglitazone, ciglitazone, pioglitazone, rosiglitazone, 5-[[4-[3-Methyl-4-oxo-3,4-dihydro-2-quinazolinyl]methoxy]phenyl-methyl]thiazolidine-2,4-dione or a pharmaceutically acceptable salt thereof, (−) 3-[4-[2-phenoxazin-10-yl ethoxy]phenyl]-2-ethoxypropanoic acid or a pharmaceutically acceptable salt thereof, miglitol, acarbose, tolbutamide, glibenclamide, glipizide, gliclazide, repaglinide, nateglinide, cholestyramine, colestipol, clofibrate, gemfibrozil, lovastatin, pravastatin, simvastatin, probucol, dextrothyroxine, alprenolol, atenolol, timolot, pindolol, propranolol and metoprolol, benazepril, captopril, analapril, fosinopril, lisinopril, quinapril, ramipril, nifedipine, felodipine, nicardipine, isradipine, nimodipine, diltiazem, verapamil, doxazosin, urapidil, prazosin or terazosin.

121. The method of claim 26, wherein said pharmaceutically acceptable composition further comprises one or more of the following: a sulphonylurea and metformin, a sulphonylurea and acarbose, repaglinide and metformin, insulin and a sulphonylurea, insulin and metformin, insulin and lovastatin or CART agonist and a CCK agonist.

122. The method of claim 118, wherein the protein tyrosine phosphatase is protein tyrosine phosphate 1B.

123. The method of claim 118, wherein the disease is diabetes, diabetes associated complications selected from hypertension, ischemic diseases of the large and small blood vessels, blindness, circulatory problems, kidney failure, and atherosclerosis; syndrome X; metabolic syndrome; glucose intolerance; insulin resistance; leptin resistance; or obesity; cancer; or neurodegenerative disease.
